SEQUENCE LISTING (59 sequences)

SEQ ID NO: 1 - DNA, 1204 bp, Oryza sativa
  CDS (9)...(989): prxrpa
tgtgagagatggagtactcttacagctacaggttcatgcttgtatgctctgttcttgtactgtgccttaatactcggggtgcgagatgccagttatccgacgatttctacgactacatatgtcctgatgtgtacaccgttgtccagcagcatgtttatgctgccatgaggactgagatgaggatgggtgcctccctcctaaggctccatttccatgactgctttgtcaatgggtgtgacggttccatccttctggacggtgacgacggcgagaaatttgcacttcccaacaagacctctgtcagagggttcgaagtcattgacgcgataaaggaagatctcgagaacatctgccctgaagttgtttcctgcgccgacattgtagcccttgcagctggctatggagtactatttagtggaggcccttactatgacgttcttctcggtagaagggatggtcttgtcgcaaatcaatcaggagctgacaacggcctcccttcaccgttcgaacccatcaaatcgatcatacagaagttcaatgatgtcggcctcgacacaaccgatgttgtcgtcctatcaggagggcacacgatcggacgagcccggtgcacgctgttcagcaaccggttgtcgaccacctcaagctcagccgacccgacgctggacgccaccatggccgccaacctccagagcctctgtgccggtggagacggcaacgagaccaccgtgctggacatcacctccgcctacgttttcgacaaccgctactaccagaacctcctcaatcagaaaggcctcctgtcctccgaccagggcctcttctccagcgacgacggcatcgccaacaccaaggagctggtggagacttacagcgcagatgcccacaagttcttctgggattttggcagatccatggtgaagatgggcaacatcagcccactcaccggtgacgacggccagattcgcaagaactgcagggttgttaattaactgagcttcagtgtgttgaaaagatagtaaatttcttgtacttttcacaaggcgattgagacgatgtgtgttatgttttgatgttacatgaatgcttgaagaagcagaagtaataacgatgtgattgtgactagtttgtgaactgatcgtcaacaaagatgtgatccattggaaaaaaagaactgaatgtatgtgccttaaaaaaaaaaaaaaaa

SEQ ID NO: 2 - protein, 326 aa, Oryza sativa
  VARIANT (1)...(326): prxrpa
MEYSYSYRFMLVCSVLVLCLNTRGARCQLSDDFYDYICPDVYTVVQQHVYAAMRTEMRMGASLLRLHFHDCFVNGCDGSILLDGDDGEKFALPNKTSVRGFEVIDAIKEDLENICPEVVSCADIVALAAGYGVLFSGGPYYDVLLGRRDGLVANQSGADNGLPSPFEPIKSIIQKFNDVGLDTTDVVVLSGGHTIGRARCTLFSNRLSTTSSSADPTLDATMAANLQSLCAGGDGNETTVLDITSAYVFDNRYYQNLLNQKGLLSSDQGLFSSDDGIANTKELVETYSADAHKFFWDFGRSMVKMGNISPLTGDDGQIRKNCRVVN

SEQ ID NO: 3 - DNA, 1218 bp, Oryza sativa
  CDS (50)...(1021): r2877
gtccatcgatcatctcttctcttccagcaaggaagcagcagagaagacaatggcagcatcggccatgaagctcgccatggcggtggcgtgcgcgctggcgctcgcgtcggcgtgccacggcctgcagctgggctactacaagcagtcgtgcccccgcgtggaggccatcgtgagggacgaggtgaagaagttcgtctacaaggacgccggcatcggcgccggactcatccgcctcgtcttccacgactgcttcgtcgagggatgtgatggctcggtgctcctggacccaactccggcgaacccgaagccggagaagctcagcccgcccaacatgcccagcctccgcggcttcgaggtgatcgacgccgccaaggacgccgtcgagaaggtctgccccggcgtggtctcgtgcgccgacatcgtcgccttcgccgcccgcgacgccgcctacttcctcagcagattcagggtcaagatcaacgtcccaggtggacgcctcgatggccgccgctccctcgactccgacgccctcaacaacctgccgccgcccaacttcaacgtgaaccagctcatcggcgcgttcgccgccaagggcctcgacgccgaggacatggtggtgctctccggcgcccacaccgtcggccgctcccactgctcctccttcgtctcggaccgcgtcgccgcgccctccgacatcaacggcggcttcgccaacttcctcaagcagaggtgcccggccaacccgacctccagcaacgacccgacggtgaaccaggacgccgtcacgcccaacgcgttcgacaaccagtactacaagaacgtggtggcgcacaaggtgctcttcgcgtcggacgcggcgctgctgacgtcgccggcgacggcgaagatggtgtcggacaacgccaacatcccggggtggtgggaggacaagttcgccaaggcgttcgtcaagatggcatccgtcggtgtcaagaccggctaccccggcgagatcaggaggcactgcagggtcgtcaactaatcatctatatattgaattcaacgtgtgttccatcaaattttctcattgctacgttttgtttttgtatttattcgttcattcttttgtgtagtgtattttgtgttgattattatatatgtcctctcctattatgtaattgtgtaatatgacactttatcatgtaataacattggaatatatttttggagtaaatttca

SEQ ID NO: 4 - protein, 323 aa, Oryza sativa
  VARIANT (1)...(323): r2877
MAASAMKLAMAVACALALASACHGLQLGYYKQSCPRVEAIVRDEVKKFVYKDAGIGAGLIRLVFHDCFVEGCDGSVLLDPTPANPKPEKLSPPNMPSLRGFEVIDAAKDAVEKVCPGVVSCADIVAFAARDAAYFLSRFRVKINVPGGRLDGRRSLDSDALNNLPPPNFNVNQLIGAFAAKGLDAEDMVVLSGAHTVGRSHCSSFVSDRVAAPSDINGGFANFLKQRCPANPTSSNDPTVNQDAVTPNAFDNQYYKNVVAHKVLFASDAALLTSPATAKMVSDNANIPGWWEDKFAKAFVKMASVGVKTGYPGEIRRHCRVVN

SEQ ID NO: 5 - DNA, 1444 bp, Oryza sativa
  CDS (108)...(1109): r1420
cgagcacttctctctcttcctgttattagctatagcagcttaattagcttaagcacaaaaggtacagtaagcctaagctagctacacactaagcaagcttattagccatggcttcatcaccaagtctgccattggtgacgtgtgccctgctgctgctgctggccgtggcatgccaggctcacccttactggccactggagttggcgtactaccgcgacaagtgcccccaggccgaggccgtcgtcaaggccgtcgtcggggaggccgtccgccagaaccccggcaatggcgccgccgtcatccgcatgctcttccacgactgctttgtcgagggttgtgatgcttcgatcctcctggacccgacgccgttcaacccgacgccagagaagctgagcgcgccgaacaacccgtccatgcggggcttcgacctcatcgacgcgatcaagcacgccgtggaggcggcgtgcccgggcgtcgtctcgtgcgccgacatcatcgccttcgcggcgcgcgacgccacctacttcctcagcggcgggaaggtctacttcgacatgccgtcgggccgccgcgacgggaccttctccaacgactccggcccgatcgacttcctcccgccgccgacgtccaacctcagcgacctcgtctccagcttcgccgtcaagggcctctccgtggaggacatggtggtgctctcgggcgcccacaccgtcggccgctcccactgctcctccttcgtccccgaccgcctcaacgcctccgtcttctccgacatcgacggcggcttcgcctggttcctcaggtcgcagtgcccgctcgacgcgacgcccggcggcaacgatcccacggtgatgctggacttcgtgacgcccaacacgctggacaaccagtactacaagaacgtgctcgaccacaaggtgctcttcacctctgacgcggcgctcctgacgtcgccggagacggcgaagatggtggtggacaacgccgtcatccccgggtggtgggaggacaggttcaaggcggccatggtgaagttggcgagcatccaggtgaagaccgggtaccaggggcagatcaggaagaactgcagggtcatcaactactgattaatctacaagtttagctgcgtgattcaattacctggtgaagtacgtgctcaacgatgacgacgtacacggtttgatagtttcacaccctccttgatcaagtcgcaagcgtacgtgttgatgatgttatgattggatgtgacttttgcttcacatttatgttctccctcttttgttaccattccttaattcctcatttgtgaaccccaaggtttacatgctatggatttctttgttttgacctaattgtgtaataaggtcatatgtacttggaataattgtaagttaagttttttgcaacaagtgcccgctatatattgatgtttatctgaaatttg

SEQ ID NO: 6 - protein, 333 aa, Oryza sativa
  VARIANT (1)...(332): r1420
MASSPSLPLVTCALLLLLAVACQAHPYWPLELAYYRDKCPQAEAVVKAVVGEAVRQNPGNGAAVIRMLFHDCFVEGCDASILLDPTPFNPTPEKLSAPNNPSMRGFDLIDAIKHAVEAACPGVVSCADIIAFAARDATYFLSGGKVYFDMPSGRRDGTFSNDSGPIDFLPPPTSNLSDLVSSFAVKGLSVEDMVVLSGAHTVGRSHCSSFVPDRLNASVFSDIDGGFAWFLRSQCPLDATPGGNDPTVMLDFVTPNTLDNQYYKNVLDHKVLFTSDAALLTSPETAKMVVDNAVIPGWWEDRFKAAMVKLASIQVKTGYQGQIRKNCRVINYL

SEQ ID NO: 7 - DNA, 1325 bp, Oryza sativa
  CDS (66)...(1046): r0317
ctcttctagccttagcctagctagctagctttgttgtctagctctgatcgaggttggtggtgatcatggcgtcgtcgagggtgatcctagcgctgctgctcgcggcggcggcggtgatggcgtcgtcggcgcagctggacgagaagttctacagcaattcgtgccccagcgtggaggccgtcgtccggaaggagatggtgcgcgcgctcggccgcgcgcccagcctcgccggcccgctcctcaggatgcacttccacgactgtttcgtcaggggttgcgacggctcggtgctgctcgactcggccgggaacagcacggcggagaaggacgcgacgccgaaccagacgctgcgcgggttcggcttcgtcgagagggtgaaggccgccgtggagaaggcatgcccgggcaccgtctcctgcgccgacgtgctcgcgctcatggccagggacgccgtgtggctgagcaagggcccgttctgggcggtgcctctcggccgccgcgacggcagggtgtccatcgccaacgagaccgaccagctgccgcctcccaccgccaacttcaccgagctcacccagatgttcgccgccaagaacctcgacctcaaggacctcgtcgtcctctccgctgggcacacgatcgggacgtcgcactgcttctccttcactgacaggctgtacaacttcaccggcctggacaacgcccacgacatcgacccgacgctggagctgcagtacatggcgaggctgaggagcaagtgcacgagcctccaagacaacacgacgctggtggagatggacccggggagcttcaagacgttcgacctggggtacttcaagaacgtggccaagcggcgggggctcttccactccgacggcgagctgctcaccaacggcttcacccgcgcctacgtgcagcgccacgccggcggcggctacaaggacgagttcttcgccgacttcgccgcctccatggtcaagatgggcggcgtcgaagtgctcaccggcagccagggcgagatcaggaagaagtgcaacgtggttaactaatcatctctcttaattatcgtcatatatcatcagctgctcgccgagactgtgattatggatttcagcttttgattccggccgtgaccatcttataattaatttccatgtagatcattatcgtaagatttacctgtcccttctttttttttcttgatttcaatcacttcattaattaattgttttgtttatatagcgagccatgcatgtatgtaacaaagttttctccaagtacgtgctcatcatgatcatatgaataaacttcattttttttcaccttgc

SEQ ID NO: 8 - protein, 326 aa, Oryza sativa
  VARIANT (1)...(326): r0317
MASSRVILALLLAAAAVMASSAQLDEKFYSNSCPSVEAVVRKEMVRALGRAPSLAGPLLRMHFHDCFVRGCDGSVLLDSAGNSTAEKDATPNQTLRGFGFVERVKAAVEKACPGTVSCADVLALMARDAVWLSKGPFWAVPLGRRDGRVSIANETDQLPPPTANFTELTQMFAAKNLDLKDLVVLSAGHTIGTSHCFSFTDRLYNFTGLDNAHDIDPTLELQYMARLRSKCTSLQDNTTLVEMDPGSFKTFDLGYFKNVAKRRGLFHSDGELLTNGFTRAYVQRHAGGGYKDEFFADFAASMVKMGGVEVLTGSQGEIRKKCNVVN

SEQ ID NO: 9 - DNA, 1317 bp, Oryza sativa
  CDS (71)...(1078): s13316
ctcaatcgtgtgaagaagctagagcataaactgcaactgataggaagctagctagctagcaggaggatcaatggcgtcgtctccgacgatgttggtggtgatgtgtagtagcttggccatggcggtgatcctgtcgtcgagctcgccggcgatggcgcagctggacgtggggttctacagcaagacgtgccccaaggtggaggagatcgtgcgggaggagatgatcaggatcctcgccgtcgcccccaccctcgccggccctctcctccgcctccatttccacgactgcttcgtcaggggttgcgacgggtcggtgctgatcgactcgacggcgagcaacacggcggagaaggacgcgccgccgaaccagacgctgcgagggttcggctccgtgcagcggatcaaggcgaggctcgacgccgcctgcccgggcaccgtctcctgcgccgacgtgctcgcgctcatggcccgcgacgccgtcgccctctccggcggcccccgctgggccgtgcccctcggccggcgagacggccgcgtctccgccgccaacgataccaccacccagctgccgccgcccaccgccaacatcacgcagctcgcccggatgttcgccgccaagggcctcgacatgaaagacctcgtcgtgctctccggcggccacacgctcggcacggcgcactgctcggcgttcacggacaggctctacaacttcaccggcgccaacaacgccggcgatgtcgaccccgccttggaccggagttatctagcgcggctccggtcgcggtgcgccagcctcgccggcgacaacacgacgctggcggagatggaccccgggagcttcctcaccttcgacgccggctactaccggctggtggcgaggcggcgggggctcttccactccgactcgtcgctgctagacgacgcgttcaccgccggctacgtgcggcggcaggccaccggcatgtacgccgccgagttcttcagggattttgccgagtcgatggtcaagatgggcggcgtcggcgtgctcaccggcggcgaaggcgagatcaggaagaagtgctacgtcatcaactaataattaatctacgtattatagcttaattaagcaattaagctcgtaattatgtgtcttaatttttcctgctgattattgattaagtgttccctaattaatcatgcgtgtaactgatgtatgtgtagagtggtggcgacatatcgatctatactagttttacttggattactttgtttgttaattgtattattatgcaatgtaactaagaaattaatcaaataatgaatcaagtattgcac

SEQ ID NO: 10 - protein, 335 aa, Oryza sativa
  VARIANT (1)...(335): s13316
MASSPTMLVVMCSSLAMAVILSSSSPAMAQLDVGFYSKTCPKVEEIVREEMIRILAVAPTLAGPLLRLHFHDCFVRGCDGSVLIDSTASNTAEKDAPPNQTLRGFGSVQRIKARLDAACPGTVSCADVLALMARDAVALSGGPRWAVPLGRRDGRVSAANDTTTQLPPPTANITQLARMFAAKGLDMKDLVVLSGGHTLGTAHCSAFTDRLYNFTGANNAGDVDPALDRSYLARLRSRCASLAGDNTTLAEMDPGSFLTFDAGYYRLVARRRGLFHSDSSLLDDAFTAGYVRRQATGMYAAEFFRDFAESMVKMGGVGVLTGGEGEIRKKCYVIN

SEQ ID NO: 11 - DNA, 1256 bp, Oryza sativa
  CDS (134)...(1108): r2151
caaacgcaggcacacaaacacagagagcagcccaaaccgaccaaaatcactgcagagttcaggcacttcttcagatagttctccccctctgtttctttctctctttatttctttctcgagttcagtttgagagatggatttggcgtggtggttcgcggtggccgtggtggtgtgcggccttgtggggggcggcagcgccgggctgctggagaccaacccgggtttggcgtacaacttctaccagaagtcgtgccccaacgtggactccatcgtccgcagcgtcacctgggcgcaggtcgccgccaacccggcgctccccggccgcctcctccgcctccacttccatgactgcttcgtccaggggtgcgacgcgtcgatcttgctggacaacgccgggagcgagaagacggcggggccgaacctatcggtggggggatacgaggtgatcgacgccatcaagacgcagctggagcaggcgtgccccggggtggtgtcgtgcgcggacatcgtggcgctcgccgcgcgcgacgccgtgtcgtaccagttcaaggcgtcgctgtggcaggtggagaccgggaggcgcgacgggcccgtgtctctggcgtccaacaccggcgcgctgccgtcgccgttcgccgggttcagcacgctcctccagagcttcgccaaccgcgggctcaacctgaccgacctcgtcgcgctctccggcgcgcacaccatcggcaaggccagctgctccagcgtcacgccgcggctgtaccaggggaacaccacctccctcgacccgctgctcgactccgcctacgccaaggcgctcatgtcgtcgtgccccaacccgtcgccgtcgtcgtccaccatcgacctcgacgtcgccacgccgctcaagttcgacagcggttactacgccaacctgcagaagaagcagggcgcgctggcgtccgacgccgcgctcacccagaacgccgccgcggcgcagatggtggcagacctcaccaacccgatcaagttctacgccgcgttctccatgtccatgaagaagatgggacgcatcgacgtgctcaccggcagcaaagggaatatcaggaagcagtgccgctccgcctcctgaatcctgacgatcaccatacaaacatattttcttcttggtttcttgaatttttttttcttcttctcctctctcgtttcatcggcgctgatcaaaatatattgtgcgccatgatgattaattaaggtcccaagatctgtattaatttgtt

SEQ ID NO: 12 - protein, 324 aa, Oryza sativa
  VARIANT (1)...(324): r2151
MDLAWWFAVAVVVCGLVGGGSAGLLETNPGLAYNFYQKSCPNVDSIVRSVTWAQVAANPALPGRLLRLHFHDCFVQGCDASILLDNAGSEKTAGPNLSVGGYEVIDAIKTQLEQACPGVVSCADIVALAARDAVSYQFKASLWQVETGRRDGPVSLASNTGALPSPFAGFSTLLQSFANRGLNLTDLVALSGAHTIGKASCSSVTPRLYQGNTTSLDPLLDSAYAKALMSSCPNPSPSSSTIDLDVATPLKFDSGYYANLQKKQGALASDAALTQNAAAAQMVADLTNPIKFYAAFSMSMKKMGRIDVLTGSKGNIRKQCRSAS

SEQ ID NO: 13 - DNA, 1156 bp, Oryza sativa
  CDS (75)...(1058): s4235
gcgtgacgcgagaggctgagaggagcattttggcccttggtttctttcggttcttctgctcttttttgctggcgatggggtggtcgtcgtcaagggctatgctcgtggcgcgcgctgtggccttggcggtggtgttcctcgcggcggaggctcagctgtcgccggggtactacaacgcgacgtgccccggggtggtgtccatcgtgcgccgcggcatggcgcaggcagtgcagaaggagtcgcgcatgggcgcctccatcctccgcctcttcttccacgactgcttcgtcaacgggtgcgacgcctccatcttgctcgacgacacggcgaacttcaccggggagaagaacgccgggccgaacgccaactcggtgcgcgggtacgaggtcatcgacgccatcaaggcgcagctcgaggcctcctgcaaggccaccgtctcctgcgccgacatcatcacgctcgccgcgcgcgacgccgtcaacctgctcgggggcccgaactggacggtgccgctggggcggcgtgacgcgcgcacgacgagccagagcgcggcgaacaccaacctgccgccgcccggcgcgagcctcgcgtcgctcctgtcgatgttcagcgccaagggcctcgacgcgcgggacctcaccgcgctgtcgggcgcgcacaccgtcgggtgggcgcgctgctccaccttccgcacgcacatctacaacgacaccggcgtgaacgccaccttcgcctcgcagctgcgcaccaagtcctgcccgaccaccggcggcgacggcaacctcgcgccgctcgagctgcaggcgcccaacaccttcgacaacgcctacttcacggacctcctcagccgccgcgtcctgctgcgctccgaccaggagctcttcggcagcggcgccggcaatggcaccacggacgcgttcgtgcgcgcgtacgccgccaacgcgacgacgttcgcggcggacttcgccgccgcgatggtgaggctgggcaacctgagcccgctcaccgggaagaacggcgaggtacggatcaactgccggcgagtgaactcatcatgaacatgaaatggattcttgatgtacgcttcgtgaacgccataaacttattatcaaatcttgtttttaccaaaagcaaatggaaaaaatgctgttcttgc

SEQ ID NO: 14 - protein, 327 aa, Oryza sativa
  VARIANT (1)...(327): s4235
MGWSSSRAMLVARAVALAVVFLAAEAQLSPGYYNATCPGVVSIVRRGMAQAVQKESRMGASILRLFFHDCFVNGCDASILLDDTANFTGEKNAGPNANSVRGYEVIDAIKAQLEASCKATVSCADIITLAARDAVNLLGGPNWTVPLGRRDARTTSQSAANTNLPPPGASLASLLSMFSAKGLDARDLTALSGAHTVGWARCSTFRTHIYNDTGVNATFASQLRTKSCPTTGGDGNLAPLELQAPNTFDNAYFTDLLSRRVLLRSDQELFGSGAGNGTTDAFVRAYAANATTFAADFAAAMVRLGNLSPLTGKNGEVRINCRRVNSS

SEQ ID NO: 15 - DNA, 1370 bp, Oryza sativa
  CDS (137)...(1147): c62847
ggcaaggagagagagagaagagagagagagagagagagagagagtaattaaggctgggaggataggcagcagcagcagcgagagggaaacgagcgagcgagcttagctggtgcttgcctataggtagctaagcaaaatggggcagaggaggaggtcggggccgcggcgtcagagccagagcgtggtggtggtggtggtcgccgtgttgctggcgacggcgtcctgcgcggcggcgcagctgagccagagctactacgcgtcgacgtgccccaacgtggagacgctcgtccgcggcgccgtcacgcagaagctcaaggagaccttcaacgccgcgcctgggacgctccgcctcttcttccacgactgcttcgtcagggggtgcgacgcgtcggtgctgatcgcggggccggacgacgagcacagcgcgggcgcggacacgacgctgtcgccggacgcgctggacctcatcacccgcgccaaggccgccgtcgacgccgacgcccagtgcgccaacaaggtctcctgcgccgacatcctcgccctcgccgcccgcgacgtcgtctcccaggcaggaggaccctactaccaggtggagctggggcggcttgacggcaaggtcgggacgcgcgccgtggtgaagcacagcctccccggcgccgccttcgacctcgaccagctcaacaagctcttcgccaccaacggcctcacccagaccgacatgatcgccctctcaggagggcacacgataggggtgacgcactgcgacaagttcgtgcggcggctgtaccagttcaagggggcggcgccgcagtacagcccgccgatgaacctggcgttcctgcggcagatgaggcagacgtgcccgctcagctacagcccgaccaccgtcgccatgctcgacgccgtctcgcccaacaagttcgacaatggctacttccagacgctgcagcagctcaagggcctcctcgcctccgaccaggtgctcttcgccgaccgccgctcgcgcgccaccgtcaactacttcgccgccaaccagaccgccttcttcgacgccttcgtcgccgccatcaccaagctcggccgcgtcggcgtcaagacggccgccggctccgacgccgagatccggcgagtctgcaccaaggtcaactaataacgtagagcagtatagctcacatttgggtggtagagagggtaaattggtgaattcttctctgtgtctctcttcttcttcttcctcttcttctccatgatgatcttcgattctcactgtccatgttcacaagtccaccgcgcacagttgttgtatttgttcccgccaattcttttcttgattcttataatcataaaaagtgtcatacattaattaggat

SEQ ID NO: 16 - protein, 337 aa, Oryza sativa
  VARIANT (1)...(337): c62847
MGQRRRSGPRRQSQSVVVVVVAVLLATASCAAAQLSQSYYASTCPNVETLVRGAVTQKLKETFNAAPGTLRLFFHDCFVRGCDASVLIAGPDDEHSAGADTTLSPDALDLITRAKAAVDADAQCANKVSCADILALAARDVVSQAGGPYYQVELGRLDGKVGTRAVVKHSLPGAAFDLDQLNKLFATNGLTQTDMIALSGGHTIGVTHCDKFVRRLYQFKGAAPQYSPPMNLAFLRQMRQTCPLSYSPTTVAMLDAVSPNKFDNGYFQTLQQLKGLLASDQVLFADRRSRATVNYFAANQTAFFDAFVAAITKLGRVGVKTAAGSDAEIRRVCTKVN

SEQ ID NO: 17 - DNA, 1138 bp, Oryza sativa
  CDS (29)...(997): r1617
gtagctagctaggtgagatcgaggggcaatggagttggtggtggcggtagcaggtgcagtagtcgtggcactgagcttgtgcattggaggtgtgcaaggtcagctccaggtagggttctacgaccaatcgtgcccccaggccgaagtgattgtgagagacgaggtcggcaaggccgtgagcgccaacgttggcctcgccgccggccttgtccggatgcacttccatgactgcttcgtcaagggatgtgacgcgtcggttttgctgtattcgacggcgaacagcacggcggagaaggacgcgataccgaacaagagcctgagggggttcgaggtggtggacagcgccaagcggcggctggagagcgcgtgcaagggggtggtctcctgcgcggacatactcgccttcgccgccagagacagcgtcgtgctggccggcggcaccccgtaccgcgttccggccgggaggagggacggcaacacctccgtggcgtccgacgcgatggccaacctgccccgtcccacctcagatgtggcccaactcacacaaagtttcgccactcatgggctttcccaggacgacatggtcatcctttcaggggcgcacacgataggggtggcgcattgcagctcgttcagctcgaggctgtacgggtacaactccagcacggggcaggacccggcgctgaacgcggcgatggcgtcgcggctgtcgcggagttgcccgcaggggagcgccaacacggtggccatggacgacggcagcgagaacacgttcgacaccagctactaccagaacctcctcgccggccgcggcgtcctcgcctccgaccagacgctcaccgccgacaacgccaccgccgcgctcgtggcgcagaacgcctacaacatgtacctcttcgccaccaagttcggccaggccatggtcaagatgggcgccatccaggtgctcaccggcagcgacggccagatccgcacaaactgcagggttgcaaactgagctaggcctcgcttgctgaacaacaacatttttgaagcctctacttgttcctatcatcagagtttttgttcttcttttgtctctatgctttaatttgtcaaacggcaaaaattcaatggaattacgcattggttttgaact

SEQ ID NO: 18 - protein, 322 aa, Oryza sativa
  VARIANT (1)...(322): r1617
MELVVAVAGAVVVALSLCIGGVQGQLQVGFYDQSCPQAEVIVRDEVGKAVSANVGLAAGLVRMHFHDCFVKGCDASVLLYSTANSTAEKDAIPNKSLRGFEVVDSAKRRLESACKGVVSCADILAFAARDSVVLAGGTPYRVPAGRRDGNTSVASDAMANLPRPTSDVAQLTQSFATHGLSQDDMVILSGAHTIGVAHCSSFSSRLYGYNSSTGQDPALNAAMASRLSRSCPQGSANTVAMDDGSENTFDTSYYQNLLAGRGVLASDQTLTADNATAALVAQNAYNMYLFATKFGQAMVKMGAIQVLTGSDGQIRTNCRVAN

SEQ ID NO: 19 - DNA, 1158 bp, Oryza sativa
  CDS (14)...(997): r3025
gagcaatatcaatatggctcagccgacttggtcagcaaggcgcgtcactgccgccctggtcgtgatggtggtggtggtgctcgcggtcgccggcgggtcgtgggcgcagctgtcgccgagcttctactcgtactcgtgcccgggagtgttcaacgcggtgaagcgggggatgcagtcggccatcgccagggagaagcgcatcggcgcctccatcgtccgcctcttcttccacgactgcttcgtccaaggttgcgacgcatcgctgctgctggacgacacggcgagcttcaccggcgagaagacggcgaaccccaacaacggctccgtcagagggtttgaggtgatcgacgccatcaagtcggcggtggagaccatctgccccggcgtcgtctcctgcgccgacatcctcgccatcgctgccagggacagcgtcgccatcctgggtgggccgagctgggacgtgaaggttggtcggagagactcgcgcacggcgagcctcagcggcgcaaacaacaacatcccgccgccgacgtcgggactcgccaacctcacctccctcttcgccgcgcaggccctctcccagaaggacatggtcgccctctccggatctcacaccattgggcaagcacgatgcacaaacttcagagctcatatatacaacgaaaccaacatcgacagtggctttgcgatgaggaggcaatcaggttgccctcgtaactcaggatcaggtgacaataacctggcacctctggatcttcagacgccaaccgtgttcgagaacaactactacaagaacctcgtcgtcaagaaggggctcctgcattctgatcaggagctcttcaatggcggagccactgatgctcttgttcagtcttacataagtagccagagcacattctttgcggattttgtgacgggcatgatcaagatgggggacatcacaccgttgacgggatcaaacggggagatcaggaagaactgcagaaggattaattaagaaatgattaagaggcaagctgtgttcatgtccagtagtgcaaacgcacattgtgtcgtgtttcttgcggatatgtttgcaacatctcccttaatttctttcatgtgtttatgtatcattatgttcgtttgaaataatgaaagtactatgtgatttgtttt

SEQ ID NO: 20 - protein, 327 aa, Oryza sativa
  VARIANT (1)...(327): r3025
MAQPTWSARRVTAALVVMVVVVLAVAGGSWAQLSPSFYSYSCPGVFNAVKRGMQSAIAREKRIGASIVRLFFHDCFVQGCDASLLLDDTASFTGEKTANPNNGSVRGFEVIDAIKSAVETICPGVVSCADILAIAARDSVAILGGPSWDVKVGRRDSRTASLSGANNNIPPPTSGLANLTSLFAAQALSQKDMVALSGSHTIGQARCTNFRAHIYNETNIDSGFAMRRQSGCPRNSGSGDNNLAPLDLQTPTVFENNYYKNLVVKKGLLHSDQELFNGGATDALVQSYISSQSTFFADFVTGMIKMGDITPLTGSNGEIRKNCRRIN

SEQ ID NO: 21 - DNA, 1230 bp, Oryza sativa
  CDS (110)...(1090): r2391
cgatcgagcttaactaattaaccatcagtagcaaattaagcctcgaccagctaatcaattaattgagcttgtgctgttagccgtgtcatctatagctcgctagcttgccatggtgtcctctgctatggcggcggtggcggtggcgtttgctgtagtggtggccgcgacgatgagcagcgcgcaacttgacccgcacttctacgacggcttgtgcccggcggcgctgcccaccatcaagcggatcgtcgaggaggccgtcgcggcggagccccgcatgggcgcctcgctcctgcgcctgcacttccacgactgcttcgtcaacggttgcgacgggtccatcctgctggacgacacgccattcttcacgggggagaagaacgcggcgcccaacatgaactccgtccgcggcttcgacgtcatcgaccgcatcaaggacgccgtcaacgccgcctgccgccgcaacgtcgtctcctgcgccgacatcgtcgccgtcgccgcgcgcgactccatcgtcaccctgggagggccgtcgtaccacgtgccgctgggccggagggacgcgcggacggcgagccaggcggcggcgaacagcagcatcccggcgccgacgctcaacctcgacggcctcgtctccagcttcgccgcgcagggcctctccgtgcaggacctcgtcctcctctccggcgcccacacgctgggcttctcccgctgcaccaacttccgcgaccgcctctacaacgagacggccacgctcgacgcctccctcgccgcgtcgctcggggggacctgcccgcgtaccgccggcgccggcgacgacaacctcgcgccgctcgacccgacgccggcgaggttcgacgccgcgtactacgcctcgctgctgcgcgccagggggctcctgcactcggaccagcagctgttcgccggcggcggcctcggcgccaccgacgggctcgtcaggttctacgccgccaacccggacgcgttccggcgagacttcgccgagtccatggtgaggatggccagcctgagcccgctcgtcgggagccaaggcgaggtccgcgtcaactgcaggaaggtgaactactactagcaagaacgcgagatggcctaaattaaatgagttccgaatgaattaatagtgcttaagtatgttaattaattaccttctcctgtggatgagagttcttcattgtggtttaattagctcatttaattcagtttgtgtgagtg

SEQ ID NO: 22 - protein, 326 aa, Oryza sativa
  VARIANT (1)...(326): r2391
MVSSAMAAVAVAFAVVVAATMSSAQLDPHFYDGLCPAALPTIKRIVEEAVAAEPRMGASLLRLHFHDCFVNGCDGSILLDDTPFFTGEKNAAPNMNSVRGFDVIDRIKDAVNAACRRNVVSCADIVAVAARDSIVTLGGPSYHVPLGRRDARTASQAAANSSIPAPTLNLDGLVSSFAAQGLSVQDLVLLSGAHTLGFSRCTNFRDRLYNETATLDASLAASLGGTCPRTAGAGDDNLAPLDPTPARFDAAYYASLLRARGLLHSDQQLFAGGGLGATDGLVRFYAANPDAFRRDFAESMVRMASLSPLVGSQGEVRVNCRKVNYY

SEQ ID NO: 23 - DNA, 1171 bp, Oryza sativa
  CDS (53)...(1033): s10927
tctgcttgcttctcttgctggttcgtcggtcggtggagtttgagcctgcgccatggctgtcggcgggagagggagacggccgctgctcctgctcctgctcctcctcgccgtggcgctggcgctggcggcgcgcgcgcgggcgcagctgtcgccggggttctactcggcgagctgccccaccgtgcacggcgtcgtgcggcaggtcatgtcgcaggccgtcatgaacgacacgcgcgccggcgccgccgtcctccgcctcttctaccacgactgcttcgtcggcggctgcgacgcgtcggtgctcctcgacgacacccccgcggcgcccggcgagaagggcgtcggccccaacgccgtcggctcgacgaccgtcttcgacctcgtcgacaccatcaaggcccaggtcgaggccgtctgccccgccaccgtctcctgcgccgacgtcctcgccatcgccgcgcgcgacagcgtcaacctgctcggcgggccgagctgggcggtgccgctcggccgccgcgacgcgctgtcgccgagtcggagcgcggtgtcgaccgacctcccgggccccgaggccgacatctccgcgctcgtctccgccttcgccgccaagggcctgagctcgcgcgacctcgccgcgctgtccggcgcgcacaccgtcggccgcgccagctgcgtcaacttccgcacccgcgtctactgcgacgccaacgtgagcccggcgttcgcgtcgcaccagcggcagtcctgcccggcgtccggcggcgacgccgcgctggcgccgctggactccctgacccccgacgcgttcgacaacggctactaccgcaacctcgtcgccggcgccgggctgctgcactccgaccaggagctgttcaacaacgggccggtggactcggtggtgcagctgtacagctccaacgccgccgccttctcgtcggacttcgccgcgtccatgatcaggctcgggaacatcggcccgttgaccgggtcaaccggcgaggtcaggctcaactgcaggaaagtgaattcctgatcgatactactagatagcattgccgcgcgcaaattgttcatagtacaaagtttaaagttatataatcagctgtcaatttcatgtattaagtgaattatgacataagaatgcaatagaaatgtcccaattttagacatc

SEQ ID NO: 24 - protein, 326 aa, Oryza sativa
  VARIANT (1)...(326): s10927
MAVGGRGRRPLLLLLLLLAVALALAARARAQLSPGFYSASCPTVHGVVRQVMSQAVMNDTRAGAAVLRLFYHDCFVGGCDASVLLDDTPAAPGEKGVGPNAVGSTTVFDLVDTIKAQVEAVCPATVSCADVLAIAARDSVNLLGGPSWAVPLGRRDALSPSRSAVSTDLPGPEADISALVSAFAAKGLSSRDLAALSGAHTVGRASCVNFRTRVYCDANVSPAFASHQRQSCPASGGDAALAPLDSLTPDAFDNGYYRNLVAGAGLLHSDQELFNNGPVDSVVQLYSSNAAAFSSDFAASMIRLGNIGPLTGSTGEVRLNCRKVNS

SEQ ID NO: 25 - DNA, 1223 bp, Oryza sativa
  CDS (20)...(982): s14493
gatatttcattttgaccatatgggttactcctactcttccgccgccgtggcggtgagcgttttggtggtggcgttggcggcggcggcgtcgggccagctgtcgacgacgttctacgcctcgtcgtgcccgaccgcgctgtcgacgatcaggagcgccgtgaacgcggcggtggccagggagccccgcatgggcgcctccctgctcaggctccacttccacgactgctttgtccaaggatgcgacgcgtcgatactgctggccgacaatgccaccttccggggggagcagggtgcgttccctaatgtcaactcgctgaggggattcgaggtcatctctagcattaagatgcaactcgaggcatcttgcaggcagaccgtctcctgcgccgacatccttgctgtcgccgcccgcgactccgtcgtcgccctaggaggtccatcgtacccggtggagctcgggaggagggacgggatgacgacgaaccaaaccatggcgaacaccaacctccatccaccgaccaccgacctgggtaacttcgtcactagcttcgccgggaaagggctcagccccaccgacctggttgtactcactggagcgcacacggtgggcgtggcgcagtgcaccaacttccggtcgcggctctacggcgagtccaacatcaacgcgccgttcgcggcgtcgctccgggcgagctgcccgcaggccggcggcgacaccaacctggcgccgctggactccacccccaacgccttcgacaacgccttcttcaccgacctcatcgccggccgcggcctcctccactccgaccaggagctctaccgcggcgacggctccggcaccgacgccctcgtccgcgtctacgccgccaaccccgcccgcttcaacgccgacttcgccgccgccatggtgcgcatgggcgccatcaggccgctcaccggcacgcagggcgagatcaggctcaactgctccagggtcaactgatctgtatatctctacgaaatgcgtcgatctactgtacctacgtggatagaatcgaatcgaataagactagactattaataagctaataagtgattagcgatatcgcgatatgtataagtgcgcaacgtgatattgaatttatgtaagggcttgtccggtttattgccattttcaacactatcgacttttaatattattattaaattttggtagaataaacgtactcttgtcattgctgcct

SEQ ID NO: 26 - protein, 320 aa, Oryza sativa
  VARIANT (1)...(320): s14493
MGYSYSSAAVAVSVLVVALAAAASGQLSTTFYASSCPTALSTIRSAVNAAVAREPRMGASLLRLHFHDCFVQGCDASILLADNATFRGEQGAFPNVNSLRGFEVISSIKMQLEASCRQTVSCADILAVAARDSVVALGGPSYPVELGRRDGMTTNQTMANTNLHPPTTDLGNFVTSFAGKGLSPTDLVVLTGAHTVGVAQCTNFRSRLYGESNINAPFAASLRASCPQAGGDTNLAPLDSTPNAFDNAFFTDLIAGRGLLHSDQELYRGDGSGTDALVRVYAANPARFNADFAAAMVRMGAIRPLTGTQGEIRLNCSRVN

SEQ ID NO: 27 - DNA, 1348 bp, Oryza sativa
  CDS (4)...(1011): prxrpn
ggcatggagtacgctactcgtggagatcgcacggctagctgcctgagtttcctctgcaatatcgtcgttctgctgggcctcgccgccgcggcgggcagcgggcagctgacggacgactactacgactattgctgcccccaggtttaccgcatcgtccggtcccgcgtggccgccgcgatgaaggccgagatgcgcatgggcgcctccctgctcaggcttcacttccacgactgcttcgtcaatggctgtgacgcgtccatcctccttgacggcacaaacagcgagaagtttgcacttcccaacaagaactcggtgagagggtacgaagtcatcgatgcgataaaggccgacctcgagggcgcctgcccgggagtcgtctcctgcgccgacatagtagcccttgcagctaaatacggagtactacttagtggaggacctgattatgatgtcctcctgggaagaagagatggtctggtggcaaatcagacgggggcgaacagtaacttgcctagccctttcgattcgatcagcgttatcactgcgaggttcaaggatgtcggtctcaacgcaaccgatgttgtggtcttatcaggggcgcacacgatcgggcgatctcgctgcctgctgttcagcaaccggctggcgaacttctcggcgaccaactccgtcgacccgacgctggactcgtcgctggcgtccagcctgcagcaggtgtgccgcggcggcgctgaccagctggcggcgctggacgtcaactccgccgacgcgttcgacaaccactactaccagaacctgctggccaacaagggcctcctcgcctccgaccagggcctcgtctccagctccggcgaccccgccgtcgccgccaccaaggcgctggtgcaggcctacagcgccaatggccagcgcttctcctgcgacttcggcaactccatggtcaagatgggcaacatcagccctctcaccggctctgccggccagattcgcaagaactgcagggccgtcaactgatgagcaaaaaggcaaagattttttgcatctgccatgaccccatcttggatttgccagaagctatatcgtcttcttggactcaagtgtgaatctgtcgtttttaatgtgttgtggagcgctacatgtttcgttttgttcaagctatctaggattctctctccaatgcacgagtagaataagcaattagcatgcaaagttgctcgtcctacacgaatctgcagctgcattttcagtgggtgtatcaccaattattagagcacagacaaacgcgctgtgtctatcagagtaaaagaaagaatatgcaactgccatatggtttaaaaaaaaaaaaaaaaaa

SEQ ID NO: 28 - protein, 335 aa, Oryza sativa
  VARIANT (1)...(335): prxrpn
MEYATRGDRTASCLSFLCNIVVLLGLAAAAGSGQLTDDYYDYCCPQVYRIVRSRVAAAMKAEMRMGASLLRLHFHDCFVNGCDASILLDGTNSEKFALPNKNSVRGYEVIDAIKADLEGACPGVVSCADIVALAAKYGVLLSGGPDYDVLLGRRDGLVANQTGANSNLPSPFDSISVITARFKDVGLNATDVVVLSGAHTIGRSRCLLFSNRLANFSATNSVDPTLDSSLASSLQQVCRGGADQLAALDVNSADAFDNHYYQNLLANKGLLASDQGLVSSSGDPAVAATKALVQAYSANGQRFSCDFGNSMVKMGNISPLTGSAGQIRKNCRAVN

SEQ ID NO: 29 - DNA, 1310 bp, Oryza sativa
  CDS (81)...(1025): r2576
cgaaaaagcttaaaaaaaactgcagttatttaggtaggattaagcattacgcgcacggcgcatcatatcgctcactctgcatggcttcggcttcttctgtcagcttaatgctgctcgtggctgcagctatggcctcagccgcctcggcgcagctgtcggcgacgttctacgacacgtcgtgcccaaacgcattgtccaccatcaagagcgcagtgacggccgccgtgaacagcgagccccgaatgggagcctcgctggtcaggctgcacttccacgactgcttcgtccaaggctgtgacgcgtctgttctgctgtccggccaggagcagaatgcaggcccgaacgctgggtcacttcggggattcaacgtcgtcgacaacatcaagacgcaggtcgaggccatctgcagccagaccgtctcctgcgccgacatcctcgccgtcgccgcccgtgactccgtcgtcgcgctcggagggccgtcgtggacggttctattgggaagaagggactccaccactgcgaacgaaagccaagcaaataccgacctccctgccccttcctctagcctcgctgaacttatcggcaatttctccagaaagggactcgacgtaaccgacatggttgctctctcaggcgcacacacgatcgggcaggcgcagtgccagaacttcagggacaggctctacaacgagacaaacatcgactccagcttcgcgacggcgctcaaggccaactgcccacggccgacgggcagcggcgacagcaacctggcgccgctggacacgacgacgcccaacgccttcgacagcgcctactacaccaacctgctgtccaacaagggcctcctgcactccgaccaggtgctcttcaacggcggcagcacggacaacacggtcaggaacttctcctccaacacggcggcgttcaacagcgccttcacggcggccatggtgaagatggggaacatctcgcccttgactggaacccaggggcagatcaggctcaactgctccaaggttaactagccggtgatgacgagtacgtgatggtctcagggaataaagctaggccccgacattgctgccaatagcgtgcagctagctgtggccatctcatcactttcatggttcaatggtttcatcagcaagtgcacggaacagactagagagtgtaggtgtatgcatgtgtgatgtcatgtaacgagttgccatgcaaaggcatgaggattacggacctctctttcgtcgtaatcctagcagctacagatttcaactgtcgtgttaaccaagtgaagtgctagtttcgaatgt

SEQ ID NO: 30 - protein, 314 aa, Oryza sativa
  VARIANT (1)...(314): r2576
MASASSVSLMLLVAAAMASAASAQLSATFYDTSCPNALSTIKSAVTAAVNSEPRMGASLVRLHFHDCFVQGCDASVLLSGQEQNAGPNAGSLRGFNVVDNIKTQVEAICSQTVSCADILAVAARDSVVALGGPSWTVLLGRRDSTTANESQANTDLPAPSSSLAELIGNFSRKGLDVTDMVALSGAHTIGQAQCQNFRDRLYNETNIDSSFATALKANCPRPTGSGDSNLAPLDTTTPNAFDSAYYTNLLSNKGLLHSDQVLFNGGSTDNTVRNFSSNTAAFNSAFTAAMVKMGNISPLTGTQGQIRLNCSKVN

SEQ ID NO: 31 - DNA, 1306 bp, Oryza sativa
  CDS (44)...(1084): r2184
cgagctgagagtgatcgatctttgttagctagagtgttgagcaatggcgtccaagctgggtatggttgtgctactgatctcgggcctttttgctgcccgttgcgcggccgtggtgaccaccggcgaacccgtcgtcgccggcctctcctgggggttctatgacacgtcgtgcccgtcggtggagggcatcgtgaggtggcacgtcaccgaggccctccgccgcgacatcggcatcgccgccggcctcgtccgcatcttcttccacgactgcttcccgcaggggtgcgacgcgtcggtcctcctgacgggttcccaaagcgagctgggtgagatacccaaccagacgctgcggccgtcggcgctgaagctcatcgaggacatccgcgccgccgtacactccgcctgcggcgccaaggtgtcctgcgccgacatcaccacgctcgccacgcgtgacgccatcgtcgcctccggcgggccctacttcgacgtgcctctggggcggcgcgacgggctggcaccggcgtcgagcgacaaggtgggcctcctgccggcgcccttcttcgacgtgcccacgctcatccaggcgttcaaggaccgaaacctggacaagacggacctggtggcgctgtccggcgcgcacaccatcggactaggccactgcggcagcttcaacgaccgcttcgatggctccaagcccatcatggaccctgtgctggtgaagaagctgcaggccaagtgcgccaaggacgtgccggtgaactcggtcacgcaggagctggacgtccgcacgcccaacgccttcgacaacaagtactacttcgacctcatcgccaagcaggggatcttcaagtccgaccagggcctcatcgaggacgcgcagaccaaccgcaccgccgtccgcttcgccctcaaccaggccgccttcttcgaccagttcgcacgctccatggtcaagatgagccagatggacgtcctcaccggcaacgccggcgagatccgcaacaactgcgccgctcccaaccgccgctcctccgacctcctcaacgctgccgacgacgaccaaggcttcgccgccgacgcctaattaacttatggagtaattagtgatcgttttatgtttatgttttgtgctagtaataataattaagaggatgccatctgcgcgtggtgtttggtttccatgcattctctgcttagttagaatggttttgcttcataaaaagtaaagttactactcgattcctcggtcgggacagagtaactgcatgtcaaatgtatcatcatcatcagttagtgctacatcatc

SEQ ID NO: 32 - protein, 346 aa, Oryza sativa
  VARIANT (1)...(346): r2184
MASKLGMVVLLISGLFAARCAAVVTTGEPVVAGLSWGFYDTSCPSVEGIVRWHVTEALRRDIGIAAGLVRIFFHDCFPQGCDASVLLTGSQSELGEIPNQTLRPSALKLIEDIRAAVHSACGAKVSCADITTLATRDAIVASGGPYFDVPLGRRDGLAPASSDKVGLLPAPFFDVPTLIQAFKDRNLDKTDLVALSGAHTIGLGHCGSFNDRFDGSKPIMDPVLVKKLQAKCAKDVPVNSVTQELDVRTPNAFDNKYYFDLIAKQGIFKSDQGLIEDAQTNRTAVRFALNQAAFFDQFARSMVKMSQMDVLTGNAGEIRNNCAAPNRRSSDLLNAADDDQGFAADA

SEQ ID NO: 33 - DNA, 1316 bp, Oryza sativa
  CDS (68)...(1114): r2693
tgatctctcacactcggatcttcttcttgattggttgcacacatcatcatcatcatcatcatcggagatggtgaagcttgtgtgctttgttgtggtggtgttcatggcggcggcggcggcgatggccggagccgatcgcgagctgaaggttgggtactacgagaagacgtgcaaggacgtggagaagatcgtgaactccatcgtcgtcaactccatcaaggacaaccgcggcaagggcgctggcctcgtccgcctcctcttccacgactgcttcgtcagggggtgtgacgcctccgtgcttctggagaagagcgagatgaacaggcagccggagaaggaatctccggcgaacatcgggatccgggggatggacgtgatcgacgcgatcaaggcggtgctggaggcgcggtgccccaacacggtgtcgtgcgccgacatcatcgcgtacgcggcgcgcgacgcgtccaggtacctcagccacggcggcgtcgacttccccgtccctgccggccgcctcgacggcgtggtctcccgcagccgcgacgccgacgcgttcctcccggacgccgccgcaaacctcaccgacctcgtccgcaacttccgccgcaagaacttcaccgtggaggagctcgtcatcctctccggcgcgcactccatcggcgtcacccactgcacctccttcgccggccgcctcaccgccccggacgcccagatcaacccgggctaccgcagcctcctcgtctccaagtgcggcggcgtgtcgccgacgccggccaacaaccacgtcgtggtgaacaacgtgcgcgacgaggacggcgccgccgtggcgagggtcatgccggggtttgcggcgagggtgaggaaggcgagggactacctggacaacagctactaccacaacaacctcgccatggcggtgacgttccacgcggactgggcgctgctcaccgggaaggaggcgcgcgggcacgtcgtggagtacgccaagaacgcgacgctgtggaacgtggacttcggcgacgcgctggtgaagctgagcaagctgcccatgccggcggggagcaagggggagatcagggccaagtgcagcgccgtcaacggctaccaccattgacgtcgacgacgatccttccatcgatcgatcgatcgaacgatctcatggatatatgatgcagtagctttgaattaatgtttgactatgatatgatctctgtaactctattgtattttttgttccttcgcccgcgtgagtactagttcaaattcaattcgttgtactgcaaacttttcaaaataattatgatgcaaagtagttc

SEQ ID NO: 34 - protein, 348 aa, Oryza sativa
  VARIANT (1)...(348): r2693
MVKLVCFVVVVFMAAAAAMAGADRELKVGYYEKTCKDVEKIVNSIVVNSIKDNRGKGAGLVRLLFHDCFVRGCDASVLLEKSEMNRQPEKESPANIGIRGMDVIDAIKAVLEARCPNTVSCADIIAYAARDASRYLSHGGVDFPVPAGRLDGVVSRSRDADAFLPDAAANLTDLVRNFRRKNFTVEELVILSGAHSIGVTHCTSFAGRLTAPDAQINPGYRSLLVSKCGGVSPTPANNHVVVNNVRDEDGAAVARVMPGFAARVRKARDYLDNSYYHNNLAMAVTFHADWALLTGKEARGHVVEYAKNATLWNVDFGDALVKLSKLPMPAGSKGEIRAKCSAVNGYHH

SEQ ID NO: 35 - DNA, 1250 bp, Oryza sativa
  CDS (31)...(1101): c52903
agccagttagagcttgcttggcaaaaagatatggccggtcagcgtgtggtggccgcggtggccgtcgctctgggcgtgtgcctgctgcagctgccggcggcgagccgcggccagctgcaggtggggttctacaacaccagctgccccaatgccgagacgctggtccggcaggccgtcaccaacgccttcgccaacgactccggcatcgccgccggcctcatccgcctccatttccacgactgcttcgtcagaggttgcgacgcgtcggtgctgctgacgtcgcccaacaacacggcggagcgcgacgcggcgccgaacaaccccagcctccgtggcttccaggtgatcgacgccgccaaggccgccgtcgagcagagctgcgcgcgcacggtgtcctgcgccgacatcgtcgcgttcgccgcccgcgacagcgtcaacctcaccggcggcgtctcctaccaggtcccctccggccgccgcgacggcaacgtctccgtcgcccaggacgccatcgacaacctcccccagcccaccttcaccgccgcccagctcgtcgccagcttcgccaacaagtcgctcaccgccgaggagatggtcgtcctctccggcgcccacaccgtcggccgctccttctgctcctccttcctcgcccgcatctggaacaacaccacccccatcgtggacacggggctgagcccggggtacgcggcgctgctgagggcgctgtgcccgtcgaacgcgtcggcgacggcgacgacggcgatcgacgtgagcacgccggcgacgctggacaacaactactacaagctgctgccgctcaacctggggctcttcttctccgacaaccagctgcgggtgaacgcgacgctgggcgcgtcggtgagcagcttcgcggcgaacgagacgctgtggaaggagaagttcgtcgccgccatggtcaagatggggagcatcgaggtgctcaccggcagccagggcgaggtcaggctcaactgcagcgtcgtcaacaaccggtcatcctcctccgccgccgggatggagacgtcgtaccactactactccggatccaccatgtccgtcgacgaggtcgcgtcgagctgatcgacgacgatgtacaacctgtttggtgttaattaggatgtgtgtatcccacgaggtcacgacgatgtagaagccgggaaggtcaaaacattaattctatgatgtatgaaaaaaagcgtgtctatgtaaaattttatatatttgtctgc

SEQ ID NO: 36 - protein, 356 aa, Oryza sativa
  VARIANT (1)...(356): c52903
MAGQRVVAAVAVALGVCLLQLPAASRGQLQVGFYNTSCPNAETLVRQAVTNAFANDSGIAAGLIRLHFHDCFVRGCDASVLLTSPNNTAERDAAPNNPSLRGFQVIDAAKAAVEQSCARTVSCADIVAFAARDSVNLTGGVSYQVPSGRRDGNVSVAQDAIDNLPQPTFTAAQLVASFANKSLTAEEMVVLSGAHTVGRSFCSSFLARIWNNTTPIVDTGLSPGYAALLRALCPSNASATATTAIDVSTPATLDNNYYKLLPLNLGLFFSDNQLRVNATLGASVSSFAANETLWKEKFVAAMVKMGSIEVLTGSQGEVRLNCSVVNNRSSSSAAGMETSYHYYSGSTMSVDEVASS

SEQ ID NO: 37 - DNA, 1233 bp, Oryza sativa
  CDS (34)...(1089): r2329
agcagctagctatagctaagctctgagcgatccatggccatgaagtgcctcttcctcttcttcgccttcctcgtcgccttcttccccggcgccgccgtcggcgccgggctgaaggtcgggttctacaacaagacgtgcccgtcggcggagcgcctggtgcagcaggcggtggccgccgcgttcaagaacaacagcggcgtcgcccccggcctcatccgcctgcacttccatgactgctttgtcagaggctgcgacgcctcggttttgatcgacgggaacgacaccgagaagaccgcgccaccaaacaaccccagcctccgcggattcgaggtgatcgacgccgccaaggccgccgtcgaggcggcgtgcccgcgtgtcgtctcctgcgccgacatcctcgccttcgccgcccgcgacagcgtcgccctcaccggcaacgtcacttacaaggtccccgccggccgccgcgacggcaacgtctccatcgcccaggacgcgctcgacaacctgccccctcccaccttcaacgccacggagctcgtcggccgcttcgccaacaagtcgctcaccgcggaggacatggtggtgctctccggcgcccacaccatcggcgtctcccactgcgactccttcaccagccgcctctacaacttcaccggcgtcggcgacgccgacccggcgatcagcgccgcctacgcgttcctcctccgcgccgtgtgcccctccaacagcagccagttcttccccaacaccacggtggacatggacgtgatcaccccggcggcgctcgacaacaagtactacgtcggggtcgccaacaacctgggcctcttcacgtcggaccacgcgctgctcaccaacgccacgctcagggcgtcggtggacgagttcgtcaagagcgagacgcggtggaagagcaagttcgtgaaggccatggtgaagatgggcggcatcgaggtgaagaccgggacgacgcagggcgaggtcaggctcaactgcagggtcgtcaacaagaggagcgccaacgctgagctcgagctcgagctcgccgccgccatggatgatggggatgaagtggcagcgagctaattaattaaggtggtagtagctgatctgatgtcgcgtgttcttcagaggtttcagcaataagtttgtgatcggtgttgttacttgttagatgtatgctgctgctgtttttaacgagaataagagttgctggagataagttttttt

SEQ ID NO: 38 - protein, 351 aa, Oryza sativa
  VARIANT (1)...(351): r2329
MAMKCLFLFFAFLVAFFPGAAVGAGLKVGFYNKTCPSAERLVQQAVAAAFKNNSGVAPGLIRLHFHDCFVRGCDASVLIDGNDTEKTAPPNNPSLRGFEVIDAAKAAVEAACPRVVSCADILAFAARDSVALTGNVTYKVPAGRRDGNVSIAQDALDNLPPPTFNATELVGRFANKSLTAEDMVVLSGAHTIGVSHCDSFTSRLYNFTGVGDADPAISAAYAFLLRAVCPSNSSQFFPNTTVDMDVITPAALDNKYYVGVANNLGLFTSDHALLTNATLRASVDEFVKSETRWKSKFVKAMVKMGGIEVKTGTTQGEVRLNCRVVNKRSANAELELELAAAMDDGDEVAAS

SEQ ID NO: 39 - DNA, 1433 bp, Oryza sativa
  CDS (52)...(1062): s11222
cacacgacacgaccgtataggagtagtcgtgtagcagctagctagctagcaatggcggcttcttcttcttcttcttcctctctggcggtggtggtggtggcggcggcggtggcgctggtcgccggcggcggcgcggcggtggcgcagctgtgcgaggagtactacgactgcacgtgccccgacgcgtacgacatcgtgcggcgggtgctgatcgatgcgcaccggagcgacgcccggatcttcgcgagcctgatccggctccatttccacgactgcttcgtgcaggggtgcgacgcgtcgctgctgctggacagcgtccccgggatgccgtcggagaagacgtcgccgcccaacaacaactccgcgaggggattccccgtcgtagacgacgtcaaggccgcgctcgaggacgcctgccccggcgttgtctcctgcgccgacatcctcgccctcgccgccgagatctccgtcgagctgtcaggtgggcccgggtggggagtgctgctggggcggctcgacggcaagacctccgacttcaatggctccctcaacctgccggcccccaccgacaacctcaccgtcctccgccaaaagttcgccgccctcaacctcaacgacgtcgacctcgtcgccctctcaggtgggcacacgttcgggagggtgcagtgccagttcgtgacggacaggctgtacaacttcagcaacacggggaggccggaccccaccatggacgccgcctaccggagcttcctgtcgcagcggtgcccgcccaacgggccgccggcggcgctcaacgacctggacccgacgacgccggacaccttcgacaaccactactacaccaacatcgaggtcaaccgcggcttcctccagtcggaccaggagctcaagtcggcgccggaggcgaccgggacgacggcgcccatcgtcgaccgcttcgccaccagccaggccgccttcttccgcagcttcgcgcagtccatgatcaacatgggcaacctctcccctgtcactgacccttccctgggtgaggtccggaccaactgcagaagggtcaattaattatcacttcatgtatgacgttggttagtttaaaattgaagtaaccaacgtcatcatgtcaaacataattaattacgtatgtgtcgattattattgttgttattattagcaaaatgagagagatttatttgtgcatagttaattaagcgcgcttgcatttgtgagaaatattagtagtccagcttaattaatcaaggagaagaggatggaaaaaagaatgtacatgtgttcaatttggatgtgtgatcaaggatctattatgatcacgcgcatccagcttaataattggccaattggttgaccaatgtaattgtacgtaccatgattgtgtttgttccaaattaaaggtgcaataaacggtttacatgttt

SEQ ID NO: 40 - protein, 336 aa, Oryza sativa
  VARIANT (1)...(336): s11222
MAASSSSSSSLAVVVVAAAVALVAGGGAAVAQLCEEYYDCTCPDAYDIVRRVLIDAHRSDARIFASLIRLHFHDCFVQGCDASLLLDSVPGMPSEKTSPPNNNSARGFPVVDDVKAALEDACPGVVSCADILALAAEISVELSGGPGWGVLLGRLDGKTSDFNGSLNLPAPTDNLTVLRQKFAALNLNDVDLVALSGGHTFGRVQCQFVTDRLYNFSNTGRPDPTMDAAYRSFLSQRCPPNGPPAALNDLDPTTPDTFDNHYYTNIEVNRGFLQSDQELKSAPEATGTTAPIVDRFATSQAAFFRSFAQSMINMGNLSPVTDPSLGEVRTNCRRVN

SEQ ID NO: 41 - DNA, 1445 bp, Oryza sativa
  CDS (74)...(1105): S14082
ctactatatgcaagttaagttagtgcactgaaggagagcctgagatacgtcgtcgtacacacttgattaattaatggcgtggaggaacagcgggcgggtgaaggtgatgaacaggaggagcagcaggatggcagtcatggcagccgccgtgctggccgtgtgcagctttgcggcggtgaccatggcccagctggagatggacttctacagcaagacgtgcccgaacgtcgaggagatcgtccggcgggagatggaggagatcctccgggtggcgccgacgctcgccggcccgctcctccgcctccatttccacgactgcttcgtcagggggtgcgacgcgtcggtgctgattgactcgacggccggcaacgtggcggagaaggacgccaagcccaacctcactctccgcggcttcggggcggtgcagcgggtcaaggacaagctcaacgccgcctgcccggccaccgtctcctgcgccgacgtcctcgccctcatggcccgtgacgccgtcgtcctcgccaacgggccctcctggcccgtctcgctcggccgccgcgacggccgcctctccatcgccaacgacaccaaccagctgccgccccccaccgccaacttcacccagctctcccagatgttcgccgccaaaggcctcgacgccaaggacctcgtcgtcctctccggcggccacacgctcggcacggcgcactgcgcgctcttctccgaccgcctctacaacttcaccggcctggtgaacgacggcgacgtggacccggcgctggacgcggcgtacatggcgaagctcaaggccaagtgccggagcctgagcgacaacaccaccctgtcggagatggaccccggcagcttcctcaccttcgacgccagctactaccggctggtggccaagcgccgcggcatcttccactccgactccgcgctgctcaccgatcccgtcaccagggcctacgtcgagcgccaggccaccggccacttcgccgacgacttcttccgcgacttcgccgactccatggtgaagatgagcaccattgacgtgctcaccggggcgcagggcgagatcaggaacaagtgctacgccatcaacatataataaagtaactgcatctgcatgcatgcgcactaatcaaaggttcgatttatttggtttcttccgtcaattttttttaattggtttcagtttatttgcaaaaaagatcgtgttaatttggtttgggtacagtacagatgaacgaatccatcactgcgtactatgtatatgtgtgtaactgatgtactatgtacgtagattgcatgccaatgggtttcttgttttgctactttttaatttgttttttggggtgcgtggcatctatgttcaactcttgtgaattcattcatgatatatgtaataagttcaaacgagacaataactaaaatatactctctttcac

SEQ ID NO: 42 - protein, 343 aa, Oryza sativa
  VARIANT (1)...(343): s14082
MAWRNSGRVKVMNRRSSRMAVMAAAVLAVCSFAAVTMAQLEMDFYSKTCPNVEEIVRREMEEILRVAPTLAGPLLRLHFHDCFVRGCDASVLIDSTAGNVAEKDAKPNLTLRGFGAVQRVKDKLNAACPATVSCADVLALMARDAVVLANGPSWPVSLGRRDGRLSIANDTNQLPPPTANFTQLSQMFAAKGLDAKDLVVLSGGHTLGTAHCALFSDRLYNFTGLVNDGDVDPALDAAYMAKLKAKCRSLSDNTTLSEMDPGSFLTFDASYYRLVAKRRGIFHSDSALLTDPVTRAYVERQATGHFADDFFRDFADSMVKMSTIDVLTGAQGEIRNKCYAINI

SEQ ID NO: 43 - DNA, 20 bp, Oryza sativa
  misc_feature (1)...(20): C52903FP1
tgtgcccgtcgaacgcgtcg

SEQ ID NO: 44 - DNA, 18 bp, Oryza sativa
  misc_feature (1)...(18): C62847FP1
tgcccgctcagctacagc

SEQ ID NO: 45 - DNA, 20 bp, Oryza sativa
  misc_feature (1)...(20): prxRPAFP1
aacctccagagcctctgtgc

SEQ ID NO: 46 - DNA, 20 bp, Oryza sativa
  misc_feature (1)...(20): prxRPARP1
aaggcacatacattcagttc

SEQ ID NO: 47 - DNA, 18 bp, Oryza sativa
  misc_feature (1)...(18): R0317F1
tcaagacgttcgacctgg

SEQ ID NO: 48 - DNA, 18 bp, Oryza sativa
  misc_feature (1)...(18): R1420FP1
ttcacctctgacgcggcg

SEQ ID NO: 49 - DNA, 20 bp, Oryza sativa
  misc_feature (1)...(20): R2184FP1
cgacaacaagtactacttcg

SEQ ID NO: 50 - DNA, 18 bp, Oryza sativa
  misc_feature (1)...(18): R2391FP2
gcctctacaacgagacgg

SEQ ID NO: 51 - DNA, 17 bp, Oryza sativa
  misc_feature (1)...(17): R2576F1
tcaaggccaactgccca

SEQ ID NO: 52 - DNA, 20 bp, Oryza sativa
  misc_feature (1)...(20): S10927FP1
cgacctcgccgcgctgtccg

SEQ ID NO: 53 - DNA, 20 bp, Oryza sativa
  misc_feature (1)...(20): S11222FP1
gacgacggcgcccatcgtcg

SEQ ID NO: 54 - DNA, 20 bp, Oryza sativa
  misc_feature (1)...(20): S13316FP1
gcgacaacacgacgctggcg

SEQ ID NO: 55 - DNA, 20 bp, Oryza sativa
  misc_feature (1)...(20): S14082FP1
tcttccactccgactccgcg

SEQ ID NO: 56 - DNA, 20 bp, Oryza sativa
  misc_feature (1)...(20): S14493FP1
cggcggcgacaccaacctgg

SEQ ID NO: 57 - DNA, 18 bp, Oryza sativa
  misc_feature (1)...(18): S4325F1
atgttcagcgccaagggc

SEQ ID NO: 58 - DNA, 20 bp, Oryza sativa
  misc_feature (1)...(20): prxRPNFP1
cctcgtctccagctccggcg

SEQ ID NO: 59 - DNA, 20 bp, Oryza sativa
  misc_feature (1)...(20): prxRPNRP1
ttaaaccatatggcagttgc

(19) United States
(12) Patent Application Publication
(10) Pub. No.: US 2004/0091860 A1
(43) Pub. Date: May 13, 2004

(54) Rice peroxidases with various characteristics

(76) Inventors: Yuko Ohashi, Ibaraki (JP); Ichiro Mitsuhara, Ibaraki (JP); Takuji Sasaki, Ibaraki (JP); Hiroyuki Ito, Sapporo (JP); Takayoshi Iwai, Ibaraki (JP); Susumu Hiraga, Sapporo (JP)

Correspondence Address: PERKINS COIE LLP, P.O. BOX 2168, MENLO PARK, CA, 94026, US

(21) Appl. No.: 10/149,506

(22) PCT Filed: Dec. 8, 2000
(86) PCT No.: PCT/JP00/08728

(30) Foreign Application Priority Data
Dec. 10, 1999 (JP) 11-352472

(51) Int. Cl. C12Q 1/68; C07H 21/04

(52) U.S. Cl. 435/6; 536/23.2

(57) ABSTRACT

The present invention relates to a plurality of peroxidases. The present invention provides clarification of the expression specificities of various peroxidases, which was conventionally difficult. The present invention further provides a means for modifying plants using the peroxidase expression specificities. The present invention also relates to peroxidases having various expression specificities and promoters derived these peroxidases. The present invention further relates to gene expression analysis using the peroxidase genes of the present invention.

### TECHNICAL FIELD

The present invention relates to a peroxidase gene of a plant. More particularly, the present invention relates to a novel peroxidase gene derived from rice. The present invention also relates to a gene analyzing method with a microarray using a group of novel peroxidase genes derived from rice, and a system and apparatus for performing the gene analyzing method.

### BACKGROUND ART

Peroxidases (EC.1.11.1.7) (also herein referred to as “POX”) are generally enzymes which catalyze oxidation of various substrates by hydrogen peroxide, and which are widely present in from microorganisms to animals and plants. Peroxidases constitute a superfamily consisting of various isozymes and isoforms, and are currently divided into class I, class II and class III, depending on the reaction specificity and structure (Welinder, Reference 1). Class I is also called prokaryote peroxidase, including yeast mitochondria cytochrome c POX, chloroplast ascorbic acid POX, cytosol ascorbic acid POX, gene bacterial POX, and the like. Class II is also called secretory fungus peroxidase, and which representatively include P. chrysosporium manganese-dependent POX (PCM), ligninase, and the like. Class III is also called classical secretory plant peroxidase, and representatively includes horseradish POX and the like. Class III plant POX is universally found in plants, and a plurality of isoforms have been found in the same plant (Reference 1).

It is suggested that class III plant peroxidase (POXs) contribute to various physiological processes in plants (e.g., lignification (Whetten et al. (Reference 2)), suberization (Espelie et al. (Reference 3)), crosslinking of cell wall proteins (Fry et al. (Reference 4)), auxin degradation and oxidization of the plant hormone indoleacetic acid (IAA) (Hinman et al. (Reference 5)), defense against pathogens (Chittoor et al. (Reference 6)), salt tolerance (Amaya et al. (Reference 7)), senescence (Abeles et al. (Reference 8)), the development, differentiation and growth of plants (Horton et al. (Reference 9)), etc.). It is also considered that class III plant peroxidase (POX) plays an important role in the growth and response to disease and wound stresses of plants, and the like. Since a plurality of peroxidases are present in a single plant and the substrate specificity thereof is low, it is also difficult to define the specific physiological functions of individual peroxidases.

As for plant POX genes, for example, at least seven POX genes have been isolated and identified from each of alfalfa, tomato and wheat (Chittoor et al. (1999), Reference 6). Chittoor et al. isolated three cDNAs and a genomic DNA fragment, which are highly homologous, from rice, and indicated that these three POXs had different induction patterns when rice is infected with Xanthomonas oryzae pv. oryzae (Chittoor et al. (1997), Reference 10). Ito et al. indicated that 25 POXs should be present in aerial parts in terms of proteins. Ito et al. purified four out of the 25, and studied the N-terminal amino acid sequences each of the four and the reactivity of each of the four with antibodies. As a result, Ito et al. inferred that the four POXs are two sets of isoforms (Ito et al., Reference 11). Ito et al. isolated cDNAs (prxRPA and prxRPN) encoding two structurally-related POXs. These two genes are not the same but exhibited similar expression patterns (Ito et al., Reference 12). It was indicated that 12 POX isozymes were detected in tobacco by isoelectric focusing followed by activity staining, and these isozymes had different organ specificities and responsiveness to wounding or TMV infection. According to the observation of these tobacco POXs, it is suggested that the expression of individual POX genes are differently regulated (Langrimini et al., Reference 13).

Thus, the number of POX isozymes which have been conventionally studied as to their expression specificities is limited. The expression tendency of POXs, the number of which is expected to be several tens, has not been analyzed in a generalized manner and it was difficult to perform such an analysis based on conventional findings.

It is known that peroxidases (POXs) have a role in removal of active oxygen species such as representatively hydrogen peroxide. In general, the state of plants or the like in which the concentration of active oxygen species (superoxide, hydrogen peroxide, hydroxyl radical, singlet oxygen, and the like) is increased in cells thereof is called oxidative stress. This is caused by a loss of balance between the oxidation state caused by a peroxidization state; ultraviolet light and radiation; abnormal conditions in the electron transfer system of cytochrome; an increase in peroxisome abnormality; non-biological causalities such as high temperature, low temperature, chemical substances, and the like; air pollutants such as ozone, sulfur dioxide, and the like; and the intracellular antioxidant protection mechanism due to the actions of superoxide dismutase (SOD), catalase (CAT), POX, vitamins E, C and A, and the like.

It has been conventionally known that if eukaryotes are exposed to an oxidative stress, the activity of SOD and the like are increased. An example has been observed, in which by inducing SOD in advance, a cell exhibited a slight level of resistance.

Further, in plants, regarding generation of oxidative stress, the following is known: (1) under various conditions for inhibiting photosynthesis (low temperature, herbicide treatment, or the like under light irradiation), active oxygen species due to a photosynthesis reaction pathway and the like are generated to an extraordinary extent; (2) also in the dark, if plants are exposed to low temperature, the concentration of active oxygen species, such as hydrogen peroxide and the like, maybe increased; and (3) active oxygen species are involved in the action mechanism of herbicide (paraquat (brand name) (1,1-dimethyl-4,4-dipyridinium dichloride) and the like) and ultraviolet light. It is believed that (4) drought stress, heavy metal, or the like cause oxidative stress.

Recently, attention has been given to the influence of environmental stresses, including oxidative stress, on organisms including plants. Modern industrialization has degraded the global environment, causing social problems. Improvement of the resistance of plants to environmental stresses has been researched. Anti-oxidization enzymes, such as POX, are believed to be a factor in resistance to environmental stresses (protection of an organism) which are useful for removal of active oxygen species excessively generated by various environmental stresses. However, the role of POX in resistance to environmental stresses has not been sufficiently clarified due to the diversity and broad substrate specificity of isozymes and the like. Therefore, at the present time, it is difficult to use POX as a material to produce plants resistant to environmental stresses.

It has been suggested that class III POX has a role in action against biological stimuli such as infection by pathogenic bacteria. In Ohashi et al. (Reference 14), the relationship between tobacco POX and generation of local lesion spots by infection of tobacco mosaic virus (TMV) was studied, and it is suggested that POX functions to oxidize polyphenolin the process of generation of lesion spots caused by necrosis due to TMV. Therefore, it is suggested that POX plays a role in conferring to plants a protection function against infection of pathogens.

Therefore, there is a demand for accumulation of detailed findings on the expression characteristics of POX genes in terms of production of useful plants including stress-resistant plants using genetic engineering methods.

Recently, a large-scale program for sequencing of expressed sequence tags (EST) is proceeding for a number of plants including rice. The presence of 42 and 41 different POX genes has been confirmed in rice (Yamamoto et al., (Reference 15)) and Arabidopsis (Østergaard et al., (Reference 16)), respectively. However, these EST sequences only provide information on the partial sequences of the genes. There has been no report on analysis of the functions of these genes.

(Problems to be Solved by the Invention)

An objective of the present invention is to provide a novel peroxidase gene group, in which the expression characteristics of each gene is clarified, and the members thereof. Another object of the present invention is to provide a plant expression promoter having identified expression specificity. The present invention is useful for clarification of a picture of the whole group of peroxidases having different expression specificities, and production of modified plants having desired traits by utilizing information obtained by such clarification. The present invention is also useful for analysis of gene expression using a DNA microarray and the like.

### DISCLOSURE OF THE INVENTION

### SUMMARY OF THE INVENTION

The present invention relates to peroxidase genes characterized by at least two expression specificities defined herein, and a set of such genes. Examples of such expression specificities include period specificity, site specificity, responsiveness to stresses, and the like. The present invention further relates to a promoter for peroxidase genes having identified expression specificity.

The present invention is based on analysis of various aspects of data on the expression specificities of 21 representative rice peroxidase genes. The present inventors clarified that individual rice peroxidase genes have separate expression patterns for various parameters (e.g., the periods of growth, tissues, and the like). Further, it was clarified that there are a plurality of rice peroxidase genes having different induced expression pattern under stresses, such as an oxygen stress, infection of pathogens, and the like.

In one aspect, the present invention relates to a set of peroxidase genes useful for evaluation of a characteristic of plants, comprising:

(A1) a subset of root-expression constitutive genes including at least one type of gene selected from the gene group consisting of:

(1) DNA having a sequence of SEQ ID NO: 1, a homolog thereof, or a fragment thereof;

(2) DNA having a sequence of SEQ ID NO: 3, a homolog thereof, or a fragment thereof;

(3) DNA having a sequence of SEQ ID NO: 5, a homolog thereof, or a fragment thereof;

(4) DNA having a sequence of SEQ ID NO: 7, a homolog thereof, or a fragment thereof;

(5) DNA having a sequence of SEQ ID NO: 9, a homolog thereof, or a fragment thereof;

(6) DNA having a sequence of SEQ ID NO: 11, a homolog thereof, or a fragment thereof;

(7) DNA having a sequence of SEQ ID NO: 13, a homolog thereof, or a fragment thereof;

(8) DNA having a sequence of SEQ ID NO: 15, a homolog thereof, or a fragment thereof;

(9) DNA having a sequence of SEQ ID NO: 17, a homolog thereof, or a fragment thereof;

(10) DNA having a sequence of SEQ ID NO: 19, a homolog thereof, or a fragment thereof; and

(11) DNA having a sequence of SEQ ID NO: 21, a homolog thereof, or a fragment thereof;

(A2) a subset of aerial-expression constitutive genes including at least one type of gene selected from the gene group consisting of:

(12) DNA having a sequence of SEQ ID NO: 23, a homolog thereof, or a fragment thereof; and

(13) DNA having a sequence of SEQ ID NO: 25, a homolog thereof, or a fragment thereof;

(B1) a subset of root-expression stress-inducible genes including at least one type of gene selected from the gene group consisting of:

(14) DNA having a sequence of SEQ ID NO: 27, a homolog thereof, or a fragment thereof;

(15) DNA having a sequence of SEQ ID NO: 29, a homolog thereof, or a fragment thereof;

(16) DNA having a sequence of SEQ ID NO: 31, a homolog thereof, or a fragment thereof;

(17) DNA having a sequence of SEQ ID NO: 33, a homolog thereof, or a fragment thereof; and

(18) DNA having a sequence of SEQ ID NO: 35, a homolog thereof, or a fragment thereof;

(B2) a subset of aerial-expression stress-inducible genes including at least one type of gene selected from the gene group consisting of:

(19) DNA having a sequence of SEQ ID NO: 37, a homolog thereof, or a fragment thereof; and

(20) DNA having a sequence of SEQ ID NO: 39, a homolog thereof, or a fragment thereof;

(C) a gene below:

(21) DNA having a sequence of SEQ ID NO: 41, a homolog thereof, or a fragment thereof.

In another aspect, the present invention relates to a peroxidase gene, wherein the peroxidase gene is any of:

(a) peroxidase DNA having a sequence of positions 50 to 1021 in SEQ ID NO: 3;

(b) peroxidase DNA having a sequence of positions 108 to 1109 in SEQ ID NO: 5;

(c) peroxidase DNA having a sequence of positions 66 to 1046 in SEQ ID NO: 7;

(d) peroxidase DNA having a sequence of positions 71 to 1078 in SEQ ID NO: 9;

(e) peroxidase DNA having a sequence of positions 134 to 1108 in SEQ ID NO: 11;

(f) peroxidase DNA having a sequence of positions 75 to 1058 in SEQ ID NO: 13;

(g) peroxidase DNA having a sequence of positions 136 to 1147 in SEQ ID NO: 15;

(i) peroxidase DNA having a sequence of positions 29 to 997 in SEQ ID NO: 17;

(j) peroxidase DNA having a sequence of positions 14 to 997 in SEQ ID NO: 19;

(k) peroxidase DNA having a sequence of positions 110 to 1090 in SEQ ID NO: 21;

(l) peroxidase DNA having a sequence of positions 53 to 1033 in SEQ ID NO: 23;

(m) peroxidase DNA having a sequence of positions 20 to 982 in SEQ ID NO: 25;

(n) peroxidase DNA having a sequence of positions 81 to 1025 in SEQ ID NO: 29;

(o) peroxidase DNA having a sequence of positions 44 to 1084 in SEQ ID NO: 31;

(p) peroxidase DNA having a sequence of positions 68 to 1114 in SEQ ID NO: 33;

(q) peroxidase DNA having a sequence of positions 31 to 1101 in SEQ ID NO: 35;

(r) peroxidase DNA having a sequence of positions 34 to 1089 in SEQ ID NO: 37;

(s) peroxidase DNA having a sequence of positions 52 to 1062 in SEQ ID NO: 39; and

(t) a gene having a sequence which hybridizes to any one sequence of (a) to (s) under stringent conditions and encoding a peroxidase having the same expression specificity as that of a peroxidase encoded by said one sequence.

In another aspect, the present invention relates to a peroxidase gene promoter, wherein the promoter is present on an upstream side of a coding region of a peroxidase gene having a sequence selected from the group consisting of SEQ ID NOs: 1, 3, 5, 7, 9, 11, 13, 15, 17, 19, 21, 23, 25, 27, 29, 31, 33, 35, 37 and 39, or a peroxidase gene which hybridizes to said peroxidase gene under stringent conditions and has the same specific expression activity as that of said peroxidase gene.

In one embodiment, the present invention relates to a peroxidase gene promoter, wherein the promoter is present on an upstream side of a coding region of a peroxidase gene having a sequence selected from the group consisting of SEQ ID NOs: 1, 3, 5, 7, 9, 11, 13, 15, 17, 19, 21, 27, 29, 31, 33 and 35, or a peroxidase gene which hybridizes to said peroxidase gene under stringent conditions and has root-specific expression activity.

In another embodiment, the present invention relates to a peroxidase gene promoter, wherein the promoter is present on an upstream side of a coding region of a peroxidase gene having a sequence selected from the group consisting of SEQ ID NOs: 15, 17, 19 and 21, or a peroxidase gene which hybridizes to said peroxidase gene under stringent conditions and has expression activity in root and aerial parts.

In still another embodiment, the present invention relates to a peroxidase gene promoter, wherein the promoter is present on an upstream side of a coding region of a peroxidase gene having a sequence selected from the group consisting of SEQ ID NOs: 23, 25, 37 and 39, or a peroxidase gene which hybridizes to said peroxidase gene under stringent conditions and has aerial-specific expression activity.

In still another embodiment, the present invention relates to a peroxidase gene promoter, wherein the promoter is present on an upstream side of a coding region of a peroxidase gene having a sequence selected from the group consisting of SEQ ID NOs: 1, 3, 5, 7, 9, 13, 15, 17, 21, 23 and 25, or a peroxidase gene which hybridizes to said peroxidase gene under stringent conditions and has constitutive expression activity.

In still another embodiment, the present invention relates to a peroxidase gene promoter, wherein the promoter is present on an upstream side of a coding region of a peroxidase gene having a sequence selected from the group consisting of SEQ ID NOs: 11 and 19, or a peroxidase gene which hybridizes to said peroxidase gene under stringent conditions and has stress reducible expression activity.

In still another embodiment, the present invention relates to a peroxidase gene promoter, wherein the promoter is present on an upstream side of a coding region of a peroxidase gene having a sequence selected from the group consisting of SEQ ID NOs: 27, 29, 31, 33, 35, 37 and 39, or a peroxidase gene which hybridizes to said peroxidase gene under stringent conditions and has stress-inducible expression activity.

In another aspect, the present invention relates to a method for producing an expression cassette, comprising the steps of:

(1) providing:

(a) a peroxidase gene containing a sequence selected from the group consisting of SEQ ID NO: 1, 3, 5, 7, 9, 11, 13, 15, 17, 19, 21, 23, 25, 27, 29, 31, 33, 35, 37 and 39; and

(b) a peroxidase gene which hybridizes to said peroxidase gene under stringent conditions and has the same specific expression activity as that of said peroxidase gene;

(2) specifying a region having a promoter activity on an upstream side of a coding region of peroxidase gene (a) or (b); and

(3) operatively linking the specified region having the promoter activity to a heterologous gene.

The peroxidase gene of the present invention may be useful for a method of producing a plant variety having a modified characteristic. For example, such a plant variety production method comprises the steps of:

preparing an expression cassette in which a promoter is operatively linked to at least one gene selected from the gene group consisting of the peroxidase genes of the present invention or homologs thereof;

introducing the expression cassette into a cell of a plant variety; and

regenerating the cell to a plant body.

In this case, the expression amount of the gene in the plant variety may be evaluated to be different from a standard expression amount of the species to which the plant variety belongs, so that the plant variety is selected (note that, in the above-described selecting step, when DNA having a sequence of SEQ ID NO: 1 or a homolog thereof, or DNA having a sequence of SEQ ID NO: 27 or a homolog thereof is selected, at least one of other genes are simultaneously selected).

Here, a “standard expression amount” of a gene refers to an average expression amount under normal growth conditions for the species to which a plant variety to be modified belongs.

The above-described production method may comprise the steps of:

introducing an expression cassette containing a promoter for a peroxidase gene of the present invention into a cell of a plant variety; and

regenerating the cell to a plant.

Examples of modification of a characteristic of a plant variety include modification of resistance to a stress caused by a factor selected from the group consisting of air pollutants, wounds, hydrogen peroxide, UV, pathogens, environmental stresses and ethylene, and further, modification of a growth characteristic or metabolism characteristic of a plant.

In another aspect, the present invention relates to a method for analyzing a characteristic of a plant using a set of peroxidase genes according to the present invention, comprising the steps of:

extracting RNA from a sample;

binding the RNA to a membrane;

labeling the set of peroxidase genes according to the present invention;

incubating the membrane along with the set of the labeled peroxidase genes; and

detecting signals derived from the labeled peroxidase genes.

The present invention also provides a method for analyzing a characteristic of a plant using a gene according to the present invention in a sample. The method comprises the steps of:

extracting RNA from a sample;

binding the RNA to a membrane;

labeling the peroxidase gene of the present invention;

incubating the membrane along with the labeled peroxidase gene; and

detecting signals derived from the labeled peroxidase gene.

In one embodiment, the characteristic is response to rice blast fungus.

In another embodiment, the gene is at least one gene selected from the group consisting of SEQ ID NOs: 29, 31, 33 and 37.

Samples in the present invention may be derived from any plants. In one embodiment, the samples may be derived from plants of the family rice.

In another embodiment, a method for analyzing a characteristic of a plant using a sequence derived from a promoter according to the present invention is provided. The method comprises the steps of:

extracting RNA from a sample;

binding the RNA to a membrane;

labeling an oligonucleotide having the sequence derived from the promoter according to the present invention;

incubating the membrane along with the labeled oligonucleotide; and

detecting a signal derived from the labeled oligonucleotide.

In another aspect, the present invention provides a method for analyzing gene expression using a DNA microarray. The method comprises the steps of:

(a) immobilizing a set of peroxidase genes according to the present invention on the DNA microarray;

(b) preparing at least two samples from a plant;

(c) labeling the samples;

(d) mixing and hybridizing the labeled samples to the DNA microarray; and

(e) washing the hybridized DNA microarray and detecting a signal derived from the label.

In another embodiment, the present invention provides a method for analyzing gene expression using a DNA microarray, comprising the steps of:

(a) immobilizing a peroxidase gene of the present invention and/or a probe of the present invention on the DNA microarray;

(b) preparing at least two samples from a plant;

(c) labeling the samples;

(d) mixing and hybridizing the labeled samples to the DNA microarray; and

(e) washing the hybridized DNA microarray and detecting a signal derived from the label.

In one embodiment, the method of the present invention further comprises the step of:

(f) correcting the detected signal.

In another embodiment, the method of the present invention further comprises the step of:

(g) analyzing the detected signal or the corrected signal by an analysis software.

In one embodiment, changes in gene expression over time may be monitored in the analysis method of the present invention using a DNA microarray. In another embodiment, a change in gene expression may be compared between plant samples given different stimuli or given no stimuli in the method of the present invention. By such comparison, global changes in gene expression over time may be monitored, or the metabolism and the like of plants may be predicted by the pattern of gene expression due to a certain stimulus.

### BRIEF DESCRIPTION OF THE DRAWINGS

FIG. 1 shows the results of phylogenetic analysis in which rice peroxidases are divided into clusters according to their putative amino acid sequences.

FIG. 2A is an electrophoresis photograph showing analysis of gene expression of rice peroxidases in growth stages and sites and due to various stimuli. Among 21 peroxidases used herein, the expression specificities of prxRPN, R2877, R1420, R0317, S13316, R2151 and S4325 are shown.

FIG. 2B is an electrophoresis photograph showing analysis of gene expression of rice peroxidases in growth stages and sites and due to various stimuli. Among 21 peroxidases used herein, the expression specificities of C62847, R1617, R3025, R2391, S10927, S14493 and prxRPA are shown.

FIG. 2C is an electrophoresis photograph showing analysis of gene expression of rice peroxidases in growth stages and sites and due to various stimuli. Among 21 peroxidases used herein, the expression specificities of R2576, R2184, R2693, C52903, R2329, S11222 and S14082 are shown.

FIG. 3 is a diagram showing the results of analysis of the expression specificity of prxRPN peroxidase. FIG. 3(a) is a graph showing the relative values of the expression amounts of mRNA in roots and aerial parts on days 5 and 16. FIGS. 3(b) and (c) shows the expression amount ratio between roots (R) and aerial parts (A) on days 5 and 16, respectively.

FIG. 4 is a diagram showing the results of analysis of the expression specificity of R2877 peroxidase. FIG. 4(a) is a graph showing the relative values of the expression amounts of mRNA in roots and aerial parts on days 5 and 16. FIGS. 4(b) and (c) shows the expression amount ratio between roots (R) and aerial parts (A) on days 5 and 16, respectively.

FIG. 5 is a diagram showing the results of analysis of the expression specificity of R1420 peroxidase. FIG. 5(a) is a graph showing the relative values of the expression amounts of mRNA in roots and aerial parts on days 5 and 16. FIGS. 5(b) and (c) shows the expression amount ratio between roots (R) and aerial parts (A) on days 5 and 16, respectively.

FIG. 6 is a diagram showing the results of analysis of the expression specificity of R0317 peroxidase. FIG. 6(a) is a graph showing the relative values of the expression amounts of mRNA in roots and aerial parts on days 5 and 16. FIGS. 6(b) and (c) shows the expression amount ratio between roots (R) and aerial parts (A) on days 5 and 16, respectively.

FIG. 7 is a diagram showing the results of analysis of the expression specificity of S13316 peroxidase. FIG. 7(a) is a graph showing the relative values of the expression amounts of mRNA in roots and aerial parts on days 5 and 16. FIGS. 7(b) and (c) shows the expression amount ratio between roots (R) and aerial parts (A) on days 5 and 16, respectively.

FIG. 8 is a diagram showing the results of analysis of the expression specificity of R2151 peroxidase. FIG. 8(a) is a graph showing the relative values of the expression amounts of mRNA in roots and aerial parts on days 5 and 16. FIGS. 8(b) and (c) shows the expression amount ratio between roots (R) and aerial parts (A) on days 5 and 16, respectively. FIG. 8(d) is a graph showing comparison of the response levels of gene expression to various stresses on day 16 with those of a control.

FIG. 9 is a diagram showing the results of analysis of the expression specificity of S4325 peroxidase. FIG. 9(a) is a graph showing the relative values of the expression amounts of mRNA in roots and aerial parts on days 5 and 16. FIGS. 9(b) and (c) shows the expression amount ratio between roots (R) and aerial parts (A) on days 5 and 16, respectively.

FIG. 10 is a diagram showing the results of analysis of the expression specificity of C62847 peroxidase. FIG. 10(a) is a graph showing the relative values of the expression amounts of mRNA in roots and aerial parts on days 5 and 16. FIGS. 10(b) and (c) shows the expression amount ratio between roots (R) and aerial parts (A) on days 5 and 16, respectively.

FIG. 11 is a diagram showing the results of analysis of the expression specificity of R1617 peroxidase. FIG. 11(a) is a graph showing the relative values of the expression amounts of mRNA in roots and aerial parts on days 5 and 16. FIGS. 11(b) and (c) shows the expression amount ratio between roots (R) and aerial parts (A) on days 5 and 16, respectively.

FIG. 12 is a diagram showing the results of analysis of the expression specificity of R3025 peroxidase. FIG. 12(a) is a graph showing the relative values of the expression amounts of mRNA in roots and aerial parts on days 5 and 16. FIGS. 12(b) and (c) shows the expression amount ratio between roots (R) and aerial parts (A) on days 5 and 16, respectively. FIG. 12(d) is a graph showing comparison of the response levels of gene expression to various stresses on day 16 with those of a control.

FIG. 13 is a diagram showing the results of analysis of the expression specificity of R2391 peroxidase. FIG. 13(a) is a graph showing the relative values of the expression amounts of mRNA in roots and aerial parts on days 5 and 16. FIGS. 13(b) and (c) shows the expression amount ratio between roots (R) and aerial parts (A) on days 5 and 16, respectively.

FIG. 14 is a diagram showing the results of analysis of the expression specificity of S10927 peroxidase. FIG. 14(a) is a graph showing the relative values of the expression amounts of mRNA in roots and aerial parts on days 5 and 16. FIGS. 14(b) and (c) shows the expression amount ratio between roots (R) and aerial parts (A) on days 5 and 16, respectively. FIG. 14(d) is a graph showing comparison of the response levels of gene expression to various stresses on day 16 with those of a control.

FIG. 15 is a diagram showing the results of analysis of the expression specificity of S14493 peroxidase. FIG. 15(a) is a graph showing the relative values of the expression amounts of mRNA in roots and aerial parts on days 5 and 16. FIGS. 15(b) and (c) shows the expression amount ratio between roots (R) and aerial parts (A) on days 5 and 16, respectively. FIG. 15(d) is a graph showing comparison of the response levels of gene expression to various stresses on day 16 with those of a control.

FIG. 16 is a diagram showing the results of analysis of the expression specificity of prxRPA peroxidase. FIG. 16(a) is a graph showing the relative values of the expression amounts of mRNA in roots and aerial parts on days 5 and 16. FIGS. 16(b) and (c) shows the expression amount ratio between roots (R) and aerial parts (A) on days 5 and 16, respectively. FIG. 16(d) is a graph showing comparison of the response levels of gene expression to various stresses on day 16 with those of a control.

FIG. 17 is a diagram showing the results of analysis of the expression specificity of R2576 peroxidase. FIG. 17(a) is a graph showing the relative values of the expression amounts of mRNA in roots and aerial parts on days 5 and 16. FIGS. 17(b) and (c) shows the expression amount ratio between roots (R) and aerial parts (A) on days 5 and 16, respectively. FIG. 17(d) is a graph showing comparison of the response levels of gene expression to various stresses on day 16 with those of a control.

FIG. 18 is a diagram showing the results of analysis of the expression specificity of R2184 peroxidase. FIG. 18(a) is a graph showing the relative values of the expression amounts of mRNA in roots and aerial parts on days 5 and 16. FIGS. 18(b) and (c) shows the expression amount ratio between roots (R) and aerial parts (A) on days 5 and 16, respectively. FIG. 18(d) is a graph showing comparison of the response levels of gene expression to various stresses on day 16 with those of a control.

FIG. 19 is a diagram showing the results of analysis of the expression specificity of R2693 peroxidase. FIG. 19(a) is a graph showing the relative values of the expression amounts of mRNA in roots and aerial parts on days 5 and 16. FIGS. 19(b) and (c) shows the expression amount ratio between roots (R) and aerial parts (A) on days 5 and 16, respectively. FIG. 19(d) is a graph showing comparison of the response levels of gene expression to various stresses on day 16 with those of a control.

FIG. 20 is a diagram showing the results of analysis of the expression specificity of C52903 peroxidase. FIG. 20(a) is a graph showing the relative values of the expression amounts of mRNA in roots and aerial parts on days 5 and 16. FIGS. 20(b) and (c) shows the expression amount ratio between roots (R) and aerial parts (A) on days 5 and 16, respectively. FIG. 20(d) is a graph showing comparison of the response levels of gene expression to various stresses on day 16 with those of a control.

FIG. 21 is a diagram showing the results of analysis of the expression specificity of R2329 peroxidase. FIG. 21(a) is a graph showing the relative values of the expression amounts of mRNA in roots and aerial parts on days 5 and 16. FIGS. 21(b) and (c) shows the expression amount ratio between roots (R) and aerial parts (A) on days 5 and 16, respectively. FIG. 21(d) is a graph showing comparison of the response levels of gene expression to various stresses on day 16 with those of a control.

FIG. 22 is a diagram showing the results of analysis of the expression specificity of S11222 peroxidase. FIG. 22(a) is a graph showing the relative values of the expression amounts of mRNA in roots and aerial parts on days 5 and 16. FIGS. 22(b) and (c) shows the expression amount ratio between roots (R) and aerial parts (A) on days 5 and 16, respectively. FIG. 22(d) is a graph showing comparison of the response levels of gene expression to various stresses on day 16 with those of a control.

FIG. 23 schematically shows a flow of a DNA microarray experiment.

FIG. 24 is a diagram showing changes in the expression patterns of the genes (R2184, R2576, R2693, C52903, R2329 and S11222) of the present invention given a stimulus of infection with rice blast fungus race (003) using three rice samples.

### BEST MODE FOR CARRAYING OUT THE INVENTION

Hereinafter, the present invention will be described in detail.

(Definitions)

A part of the major terms used herein will be defined below.

“Plant” as used herein includes any of the monocotyledons and dicotyledons. Examples of preferable plants include monocotyledons belonging to the family rice, such as wheat, maize, rice, barley, Sorghum, and the like. Other examples of preferable plants include tobacco, pimento, eggplant, melon, tomato, sweet potato, cabbage, onion, broccoli, carrot, cucumber, citrus, Chinese cabbage, lettuce, peach, potato, and apple. Preferable plants are not limited to crops, but include flowers, trees, grasses, weeds, and the like. Plant means any of a plant itself, plant organs, plant tissues, plant cells, and seeds unless otherwise specified. Examples of plant organs include root, leaf, stem, flower, and the like. Examples of plant cells include callus and suspension cultured cells. “Fragment” of DNA as used herein refers to a polynucleotide having a length which is shorter than the full length of the reference DNA but sufficient for use at least as a probe or a primer. A certain DNA fragment has to be capable of specifically hybridizing in order to be used as a selective probe or a selective primer for DNA from which the fragment originated. “A certain DNA hybridizes specifically to” as used herein indicates that when peroxidases (POXs) are used, at least 21 POX DNAs of the present invention can be separately detected and amplified. The selective probe may have a length of representatively at least 10 nucleotides, preferably at least 15 nucleotides, more preferably at least 20 nucleotides, and even more preferably at least 30, 40 or 50 nucleotides, and may further have a length of more than 50 nucleotides. The selective probe may be available as a product of PCR amplification using a selective primer. When a selective primer is used as at least one of a pair of primers in PCR, the selective primer has a length of representatively at least 9 nucleotides, preferably at least 10 nucleotides, more preferably at least 15 nucleotides, even more preferably at least 17, 18, 19, 20, 21, 22, 23, 24, 25, 30 or 50 nucleotides, or more than 50.

Herein, in order for a fragment of each POX to be specific, the fragment has to be selected from regions excluding a POX conserved region. “POX conserved region” as used herein refers to a region in which a DNA sequence or an amino acid sequence is conserved between the POXs of the present invention. “POX non-conserved region” refers to other than such a region. “Conserved” indicates that the sequence of a certain nucleic acid sequence region is the same as or similar to the original nucleic acid sequence to an extent that the functions of a polypeptide encoded by the sequence are retained. The POX conserved regions are representatively portions of a sequence alignment indicated by boxes in the sequence below. These portions are characterized as regions including particularly two invariable histidine (represented by h) residues and 8 cysteine residues (represented by c1 to c8). The invariable histidine residues in the POX conserved regions correspond to amino acids 67 and 193, respectively, in prxRPA (SEQ ID NO: 28), for example. The invariable cysteine residues in the POX conserved regions correspond to amino acids 38, 71, 76, 115, 121, 200, 230 and 322, respectively, in prxRPA (SEQ ID NO: 28), for example.

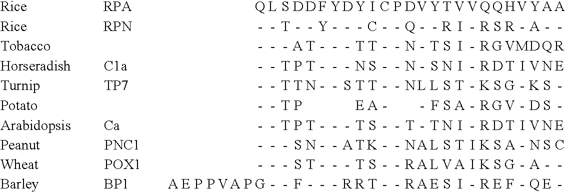

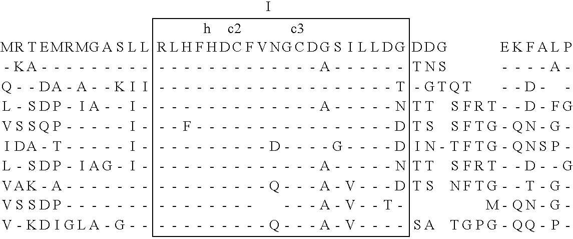

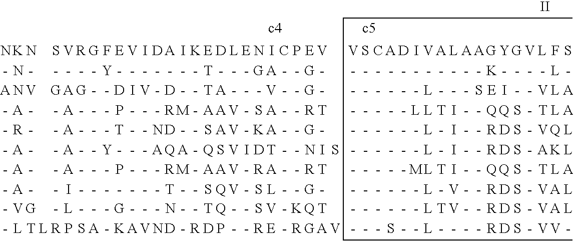

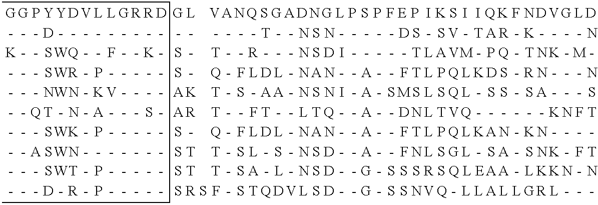

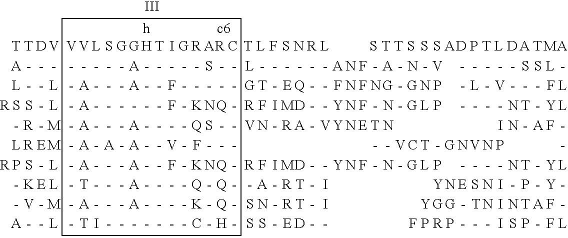

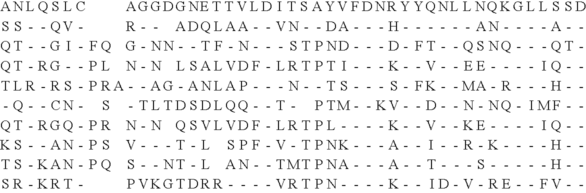

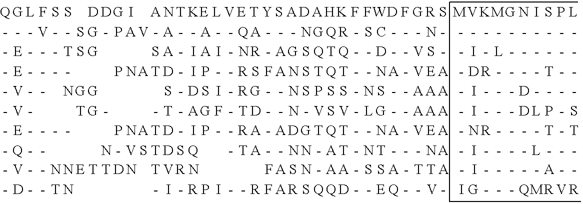

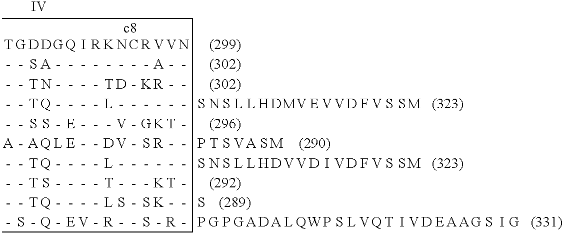

“Homolog” of DNA as used herein refers to DNA having a nucleotide sequence which is homologous to the nucleotide sequence of a reference DNA. Representatively, homolog refers to a polynucleotide which hybridizes to a reference DNA under stringent conditions. In the case of peroxidases (POXs), a “homolog” of a POX gene is DNA which has a DNA sequence sharing homology with the DNA sequence of the POX gene, and has the same or similar expression characteristics (e.g., site specificity, period specificity, responsiveness to stresses, and the like).

Herein, a homolog of a certain POX gene generally has homology to the POX non-conserved region of a POX polypeptide to be referenced, but not to the POX non-conserved region of the other POX polypeptide. “Homology” of a gene refers to the magnitude of identity between two or more gene sequences. Therefore, the greater the homology between two genes, the greater the identity or similarity between their sequences. Whether or not two genes have homology is determined by comparing their sequences directly or by a hybridization method under stringent conditions. When two gene sequences are directly compared with each other, the genes have homology if representatively at least 50%, preferably at least 70%, more preferably at least 80%, 90%, 95%, 96%, 97%, 98%, or 99% of the DNA sequence of the genes are identical.

Comparison in identity between base sequences may be calculated using a sequence analysis tool, FASTA (Pearson et al., Reference 17), for example.

“A POX gene has ‘the same or similar’ expression characteristics” indicates that at least one of the site specificity, period specificity and responsiveness to stresses of expression, preferably any two of the characteristics, more preferably all of the characteristics are the same as or similar to each other. When site specificity is herein mentioned, the proportions of the expression amount of a POX gene in roots and aerial parts are evaluated. POX genes are divided into three groups: expression is predominant in the root; expression is predominant in the aerial parts; and expression is substantially of the same level between the roots and the aerial parts. In this case, when genes are categorized into the same group, the genes are said to be “the same as or similar to” each other. When period specificity is mentioned, the ratio of the expression amounts of a POX gene on day 5 and day 16 of germination is evaluated. POX genes are divided into three groups: expression is predominant on day 5 (juvenile period); expression is predominant on day 16 (mature period); and expression is substantially of the same level in both periods. In this case, when genes are categorized into the same group, the genes are said to be “the same as or similar to” each other. When responsiveness to stresses is mentioned, a change in the expression amount of a POX gene is evaluated when a plant is subjected to any of stresses due to chemicals (e.g., paraquat, ethephon, methyl jasmonate (MeJA), and the like) and physical stimuli (e.g., ultraviolet light (UV), cutting, rubbing, and the like). POX genes are divided into three groups, depending on responsiveness to a particular stress: the expression amount is increased; the expression amount is decreased; and the expression amount is not changed. In this case, when genes are categorized into the same group, the genes are said to be “the same as or similar to” each other. To investigate each of the above-described expression characteristics, the expression amounts of POX genes may be confirmed by northern blot analysis under conditions similar to those in the examples below.

“Stringent conditions” for hybridization as used herein refer to conditions under which the complementary strand of a nucleotide strand having homology to a target sequence predominantly hybridizes the target sequence, and the complementary strand of a nucleotide strand having no homology substantially does not hybridize. “Complementary strand” of a certain nucleic acid sequence refers to a nucleic acid sequence paired with the certain nucleic acid sequence by hydrogen bonds between nucleic acid bases (e.g., T for A and C for G). The stringent conditions are sequence-dependent, and vary depending on various circumstances. The higher the sequence, the higher temperature the sequence specifically hybridizes at. In general, as for the stringent conditions, the temperature is selected about 5° C. lower than a heat melting point (Tm) of a particular sequence at a predetermined ionic strength and pH. Tm is the temperature at which 50% of nucleotides complementary to a target sequence hybridize to the target sequence in an equilibrium state under a predetermined ionic strength, pH, and nucleic acid concentration. “Stringent conditions” are sequence-dependent and vary depending on various environmental parameters. General guidelines for hybridization of nucleic acids can be found in Tijssen (Reference 18).

Representatively, as to the stringent conditions, the salt concentration is less than about 1.0 M Na⁺, representatively about 0.01 to 1.0 M Na⁺ concentration (or other salts) at pH 7.0 to 8.3, and the temperature is at least about 30° C. for short nucleotides (e.g., 10 to 50 nucleotides) and at least about 60° C. for long nucleotides (e.g., 50 nucleotides). The stringent conditions may be achieved by addition of a destabilizer, such as formamide. Herein, examples of the stringent conditions include hybridization in buffered solution containing 50% formamide, 1 M NaCl, and 1% SDS (37° C.), and washing with 0.1×SSC at 60° C.

Herein, when used regarding the expression of peroxidase (POX) in plants, “site specificity” generally refers to the expression specificity of a POX gene to a site of a plant (e.g., root, stem, trunk, leaves, flower, seed, germ, embryo, fruit, and the like). “Period specificity” refers to the expression specificity of a POX gene to a growth stage of a plant (e.g., the number of days after germination of a seedling). “Responsiveness to stresses” refers to a change in the expression of a POX gene caused by at least one stress given to a plant.

Here, “stress” may be a factor which is physically, chemically or biologically applied to plants which are in turn inhibited from growing normally. Examples of stresses include physical stresses (light, heat, cooling, freezing, ultraviolet light, X-ray, cutting, rubbing, and the like), chemical stresses (oxygen stress, chemicals, biologically active substances, and the like), biological stresses (viruses, pathogens (e.g., rice blast fungus infection)), and the like. When herein used, “environmental stresses” refer to stresses to plants caused by changes in the global environment. For example, the stresses are caused mainly by an increase in the amount of ultraviolet light due to the destruction of the ozone layer, and active oxygen species and chemicals due to air pollution, or the like.

“Oxygen stress” or “oxidative stress” refers to a stress caused by oxygen and derivatives of oxygen, representatively, active oxygen species (superoxide, hydrogen peroxide, hydroxyl radical, singlet oxygen, and the like), ozone, air pollutants (e.g., SOₓ, NOₓ, and the like), and the like. The oxidative stress is caused by loss of a balance between an “oxidation state” caused by a peroxidization state; ultraviolet light or radiation; abnormal conditions in the electron transfer system of cytochrome; an increase in peroxisome abnormality; non-biological causalities such as high temperature, low temperature, chemical substances, and the like; air pollutants such as ozone, sulfur dioxide, and the like; and the intracellular “antioxidant protection mechanism” due to the actions of superoxide dismutase (SOD), catalase (CAT), POX, vitamins E, C and A, and the like.

“Root expression type” as used herein refers to any of a trait in which a POX gene or a promoter therefor is expressed predominantly in the root of a plant, and a trait in which a POX gene or a promoter therefor is similarly expressed in the roots or aerial parts of a plant. Particularly, the trait in which a POX gene or a promoter therefor is similarly expressed in the roots or aerial parts of a plant is called a “root and aerial part expression type”. “Aerial part expression type” refers to a trait in which a POX gene or a promoter therefor is expressed in at least a portion of the aerial parts of a plant more predominantly than the roots. These traits can be determined by extracting RNA from each portion and subjecting the RNA to northern blot analysis to analyze expression amounts.

“Structural” expression of a POX gene or a promoter therefor as used herein refers to a trait in which expression is similarly carried out in a plant tissue during the juvenile period and the mature period in the course of the growth of a plant. Specifically, when northern blot analysis is carried out under conditions similar to those in the examples described herein, if expression is observed in the same or corresponding site of a seedling on both day 5 and day 16, the expression is regarded as being constitutive by the definition in the present invention. Structural peroxidases are believed to play a role in the homeostasis of plants in a normal growth environment. “Responsiveness to stresses” expression of a POX gene or a promoter therefor refers to a trait in which when at least one stress is applied to a plant, the expression amount is changed. Particularly, a trait in which the expression amount is increased is called “stress inductivity”, and a trait in which the expression amount is decreased is called “stress reducibility”. “Stress reducible” expression is based on the assumption that expression can be observed in normal cases, and therefore overlaps the idea of “constitutive” expression. These traits can be determined by extracting RNA from an arbitrary portion and subjecting the RNA to northern blot analysis to analyze expression amounts.

(Various Rice Peroxidases)

The present inventors indicated that a number of rice peroxidase genes have different expression specificities. Herein, rice peroxidases may be divided representatively into at least 5 classes, A1, A2, B1, B2 and C, depending on the expression specificity. Categories A and B are based on the responsiveness (inducibility) to stimuli. POXs having no inducibility to stresses are categorized as class A and POXs having inducibility to stresses are categorized as class B. POXs having an expression level which is no more than the limit of detection are categorized as class C.

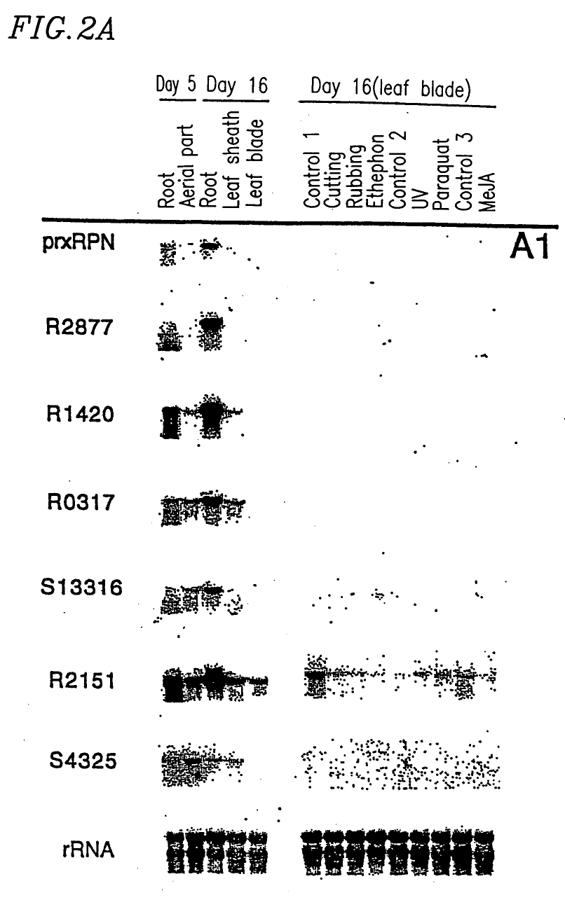

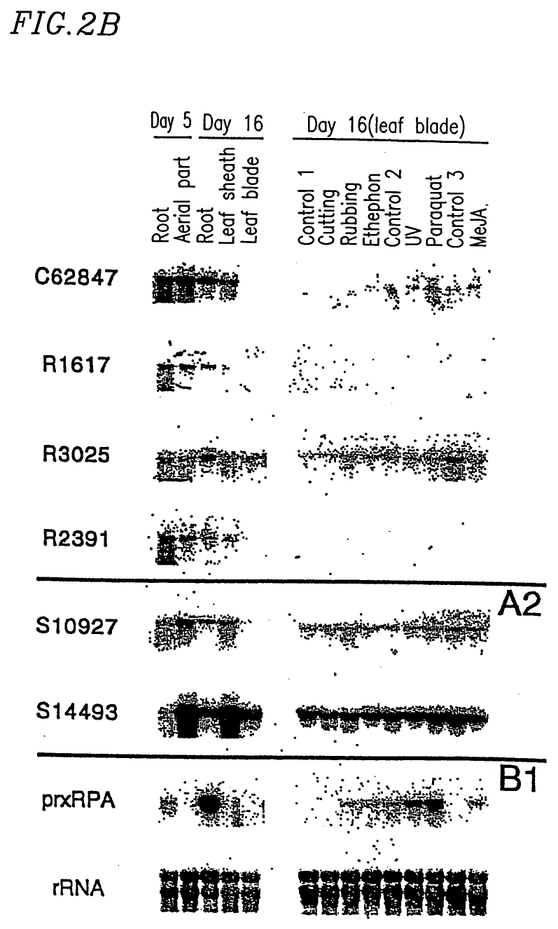

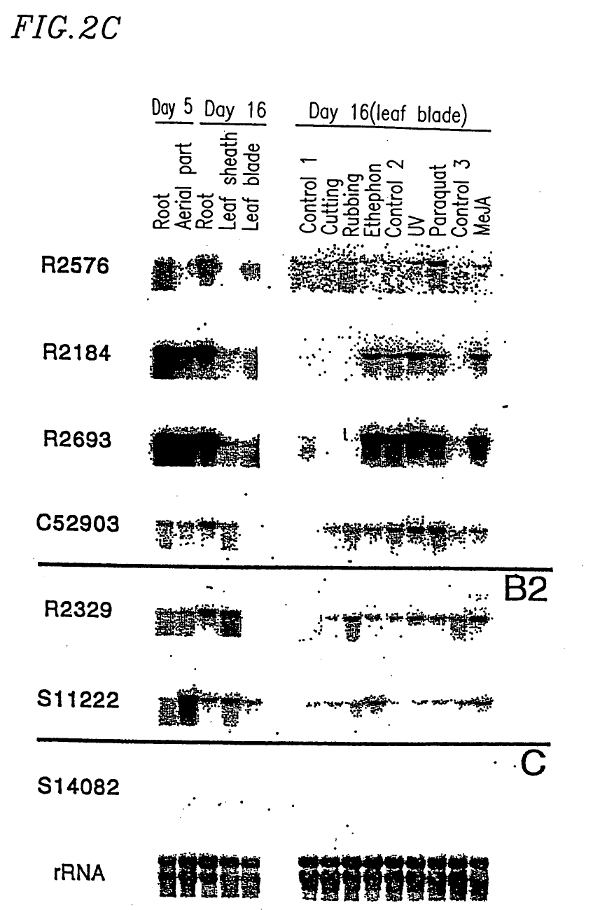

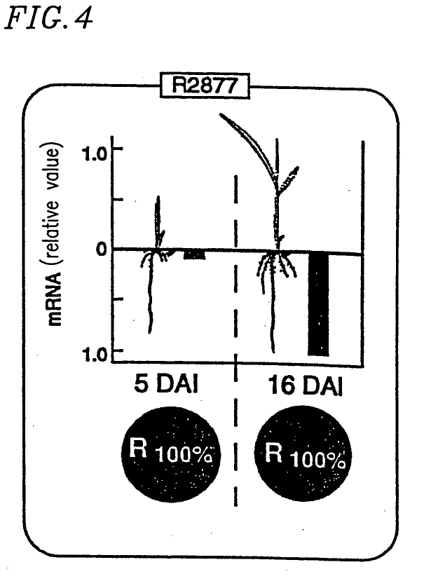

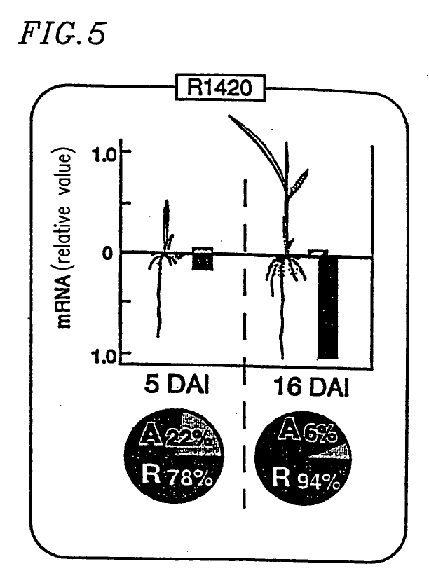

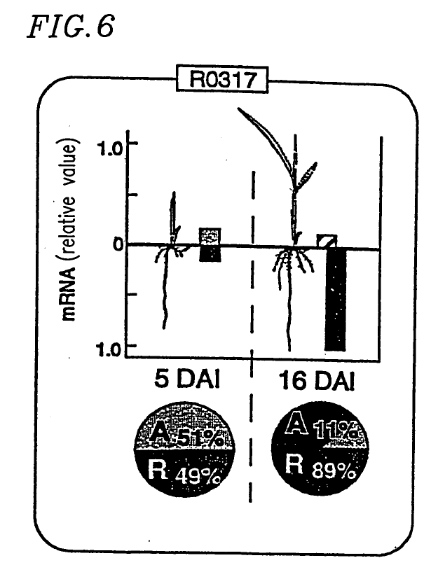

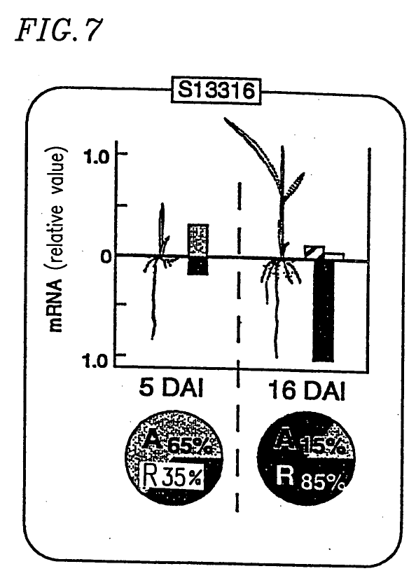

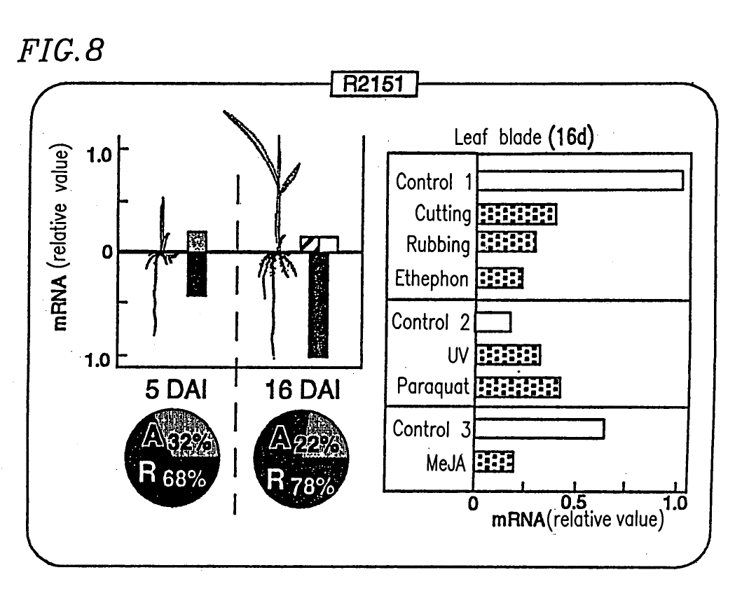

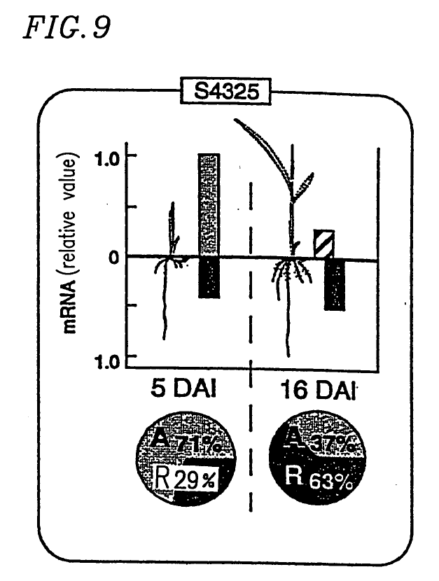

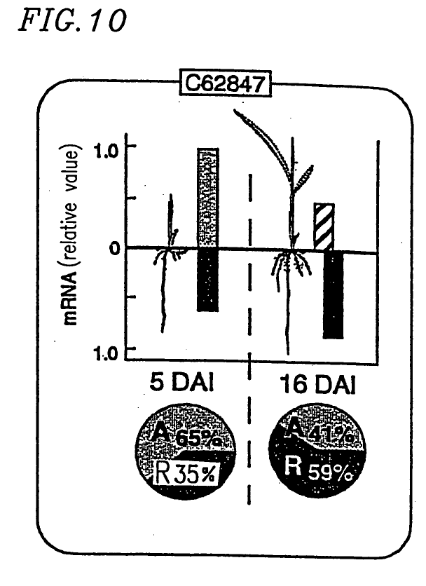

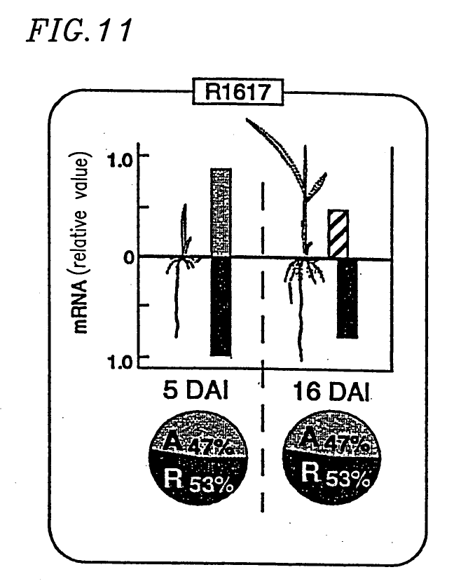

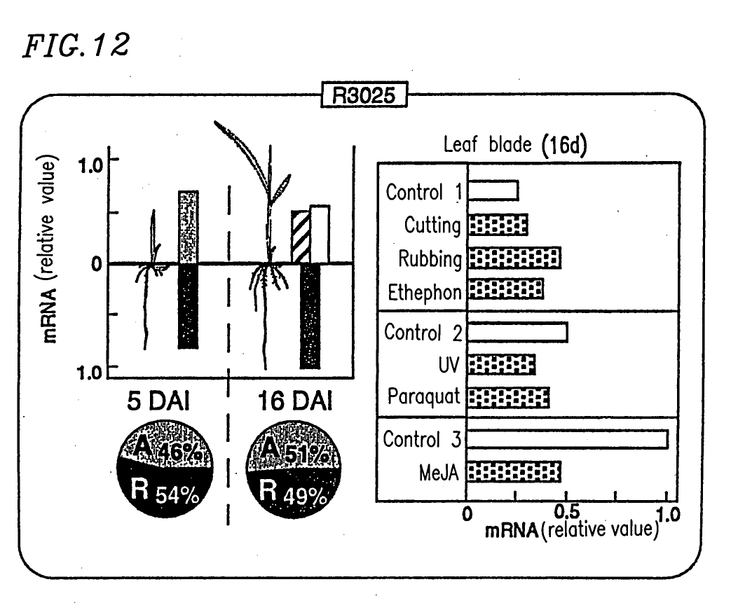

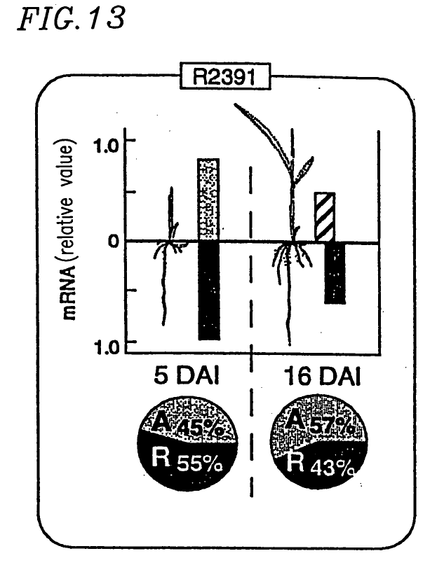

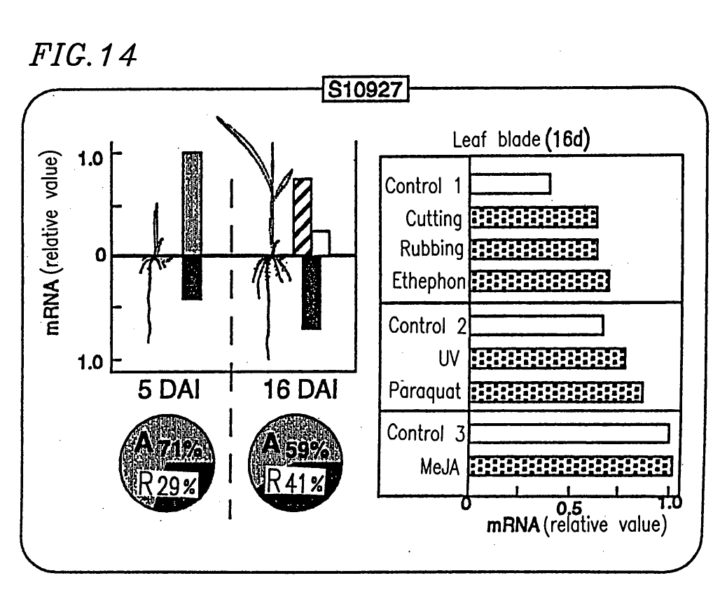

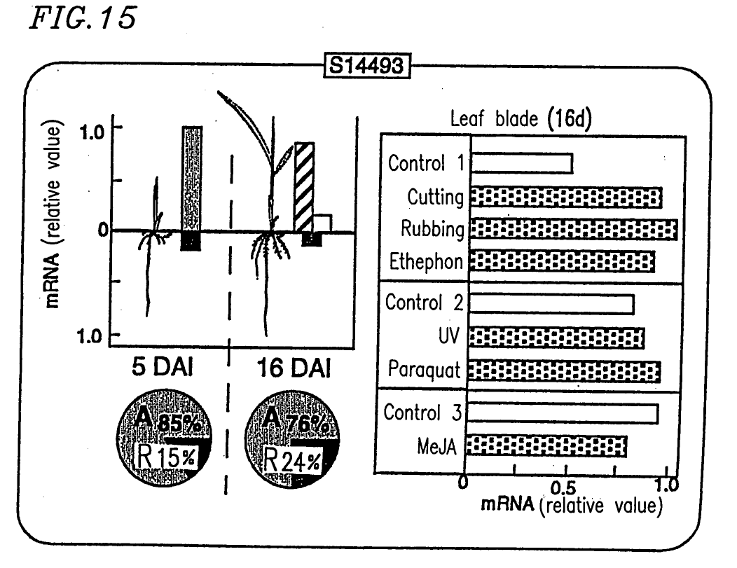

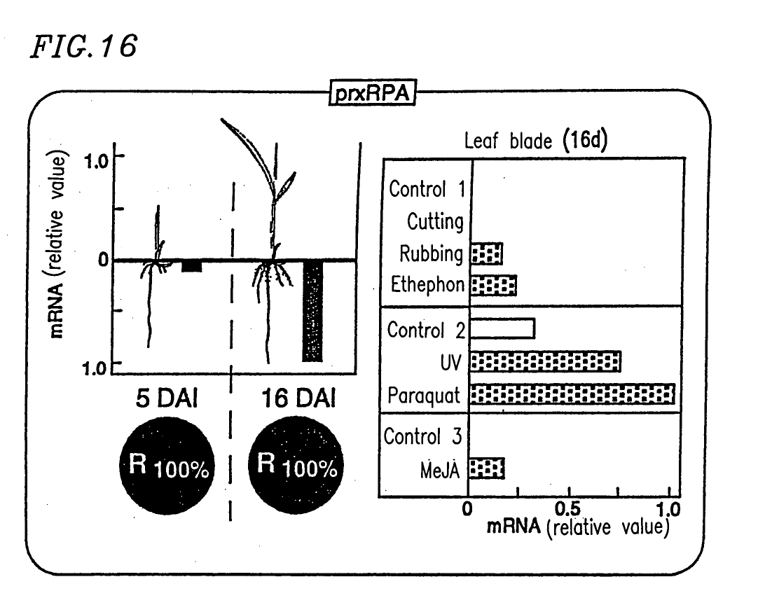

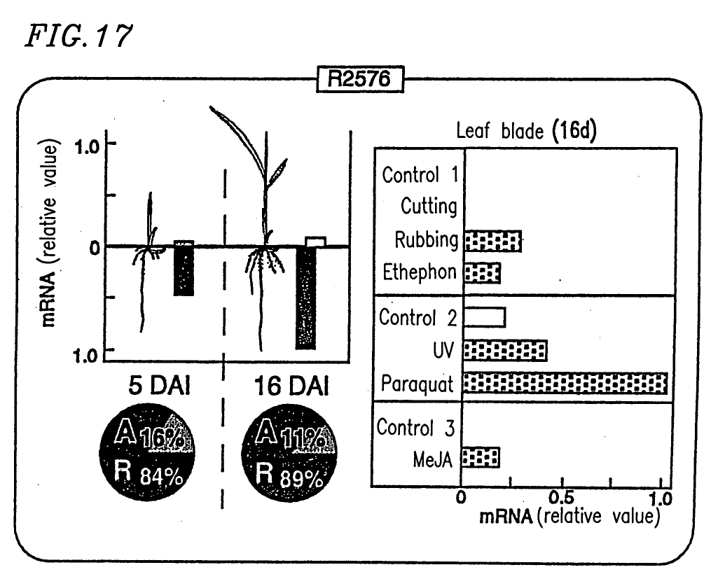

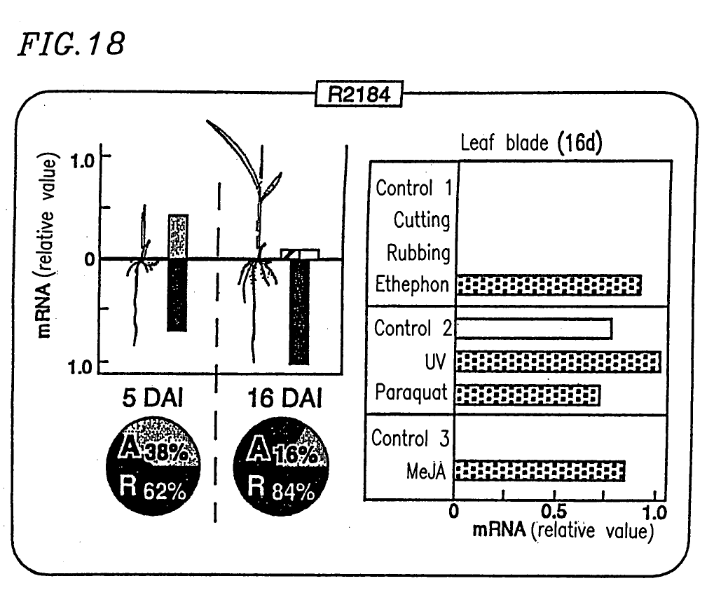

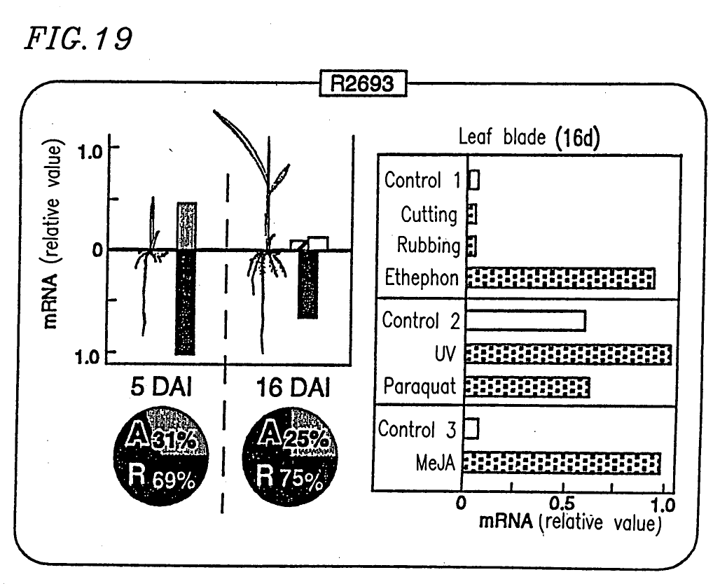

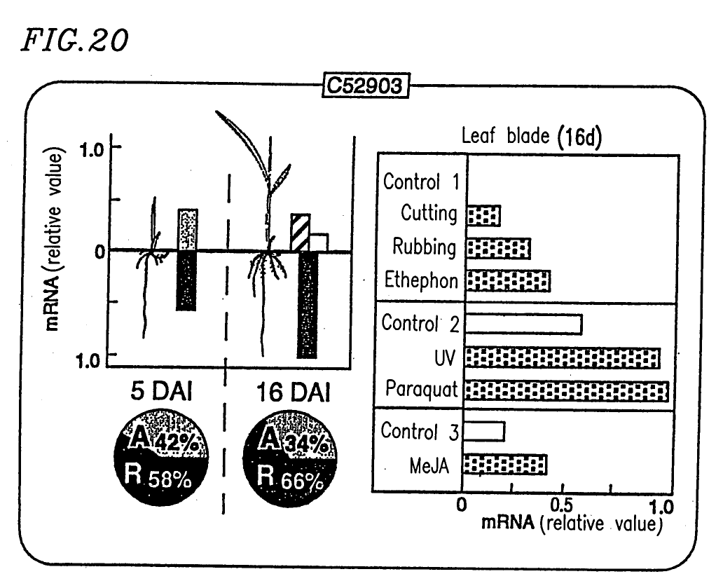

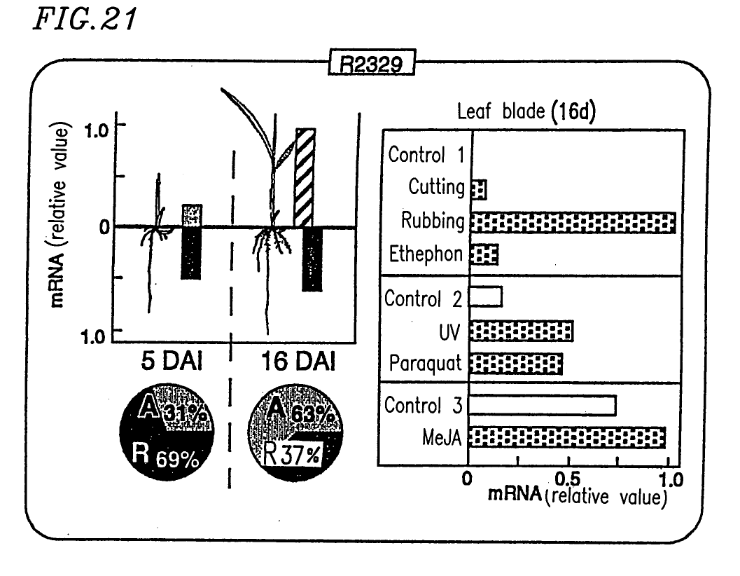

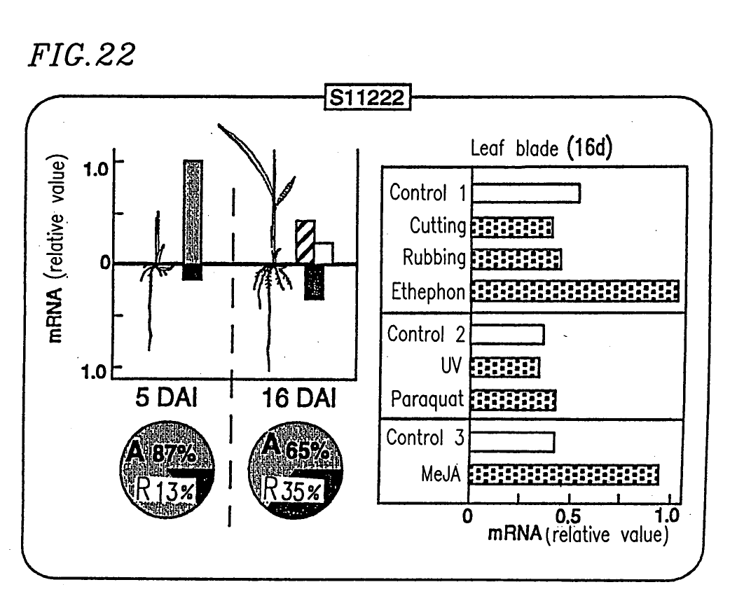

POXs of the “root expression type” or the “root and aerial part expression type”, which are expressed predominantly in below ground parts or similarly in below-and aboveground parts, are categorized as A1 and B1. POXs which are expressed mainly in aerial parts are categorized as A2 and B2. (e.g., see FIGS. 2A to 2C and 3 to 22; the summary of the categorization is shown in Table 1.)

The present inventors analyzed expression of the 21 POX genes. As a result, 16 enzymes were categorized as A1 and B1, 4 enzymes were categorized as A2 and B2, and one enzyme was categorized as C. It was clarified that the POX genes of the present invention exhibit various, different and separate responsivenesses to stresses, and are expressed mainly in roots rather than aerial parts, as described below in detail. This was difficult to predict from conventional preliminary findings (Reference 11 and the like).

TABLE 1

| | Induction by | Spatial | |
|---|---|---|---|
| Group | stimuli^{a} | distribution^{b,c} | Number^{d} |
| A1 | no | root ≧ aerial | 11 |
| | | part | |
| A2 | no | root < aerial | 2 |
| | | part | |
| B1 | yes | root ≧ aerial | 5 |
| | | part | |
| B2 | yes | root < aerial | 2 |
| | | part | |
| C | no detection | no detection | 1 |

^{a}leaf blade of 16-day-old plants
^{b}16-day-old plants
^{c}root ≧ aerial part where the ratio of the root to the aerial part exceeds 4/6
^{d}the number of POX genes in the group

Hereinafter, representative rice POX genes within the scope of the present invention will be described.

prxRPN as well as prxRPA described below are POXs which have been isolated by the present inventors (Ito et al. (1994), Reference 12). These sequences have been isolated from rice based on a sequence conserved in plant peroxidases. The prxRPN gene has a sequence indicated by SEQ ID NO: 1. The genetic products of this gene are categorized as A1 because of the expression specificity thereof. The genetic products are expressed predominantly in roots. This suggests that the prxRPN gene has a function contributing to characteristics of plants, such as an ability to grow under anaerobic conditions, such as under water and the like. Examples of a primer having a sequence specific to this gene include prxRPNFP1 and prxRPNRP1 (SEQ ID NO: 58 and 59). These primers are useful for obtaining a gene of interest or genes similar thereto. A promoter derived from the prxRPN gene is considered to reflect the expression specificity of prxRPN. Use of the prxRPN gene and homologs thereof and promoters derived therefrom is useful for modification of plant characteristics.

R2877 is a novel POX obtained from rice based on the EST sequences, and belongs to A1 in accordance with the categorization described herein. This gene has a sequence indicated by SEQ ID NO: 3. R2877 may be expressed predominantly in roots. R2877 is expressed in a seedling during the mature period more significantly than during the juvenile period. Dominant expression of genetic products in roots suggests that R2877 has a function contributing to characteristics of plants, such as an ability to grow under anaerobic conditions, such as under water and the like. The significant expression in a seedling during the mature period suggests that R2877 is required for the metabolism of a plant during the mature period, such as regulation of the amount of indoleacetic acid (IAA) which is a plant hormone, and the like. A promoter derived from the R2877 gene is considered to reflect the expression specificity of R2877. Use of the R2877 gene and homologs thereof and promoters derived therefrom is useful for modification of plant characteristics.

R1420 is also a sequence obtained from rice based on the EST sequences, and belongs to A1 in accordance with the categorization described herein. This gene has a sequence indicated by SEQ ID NO: 5. R1420 may also be expressed predominantly in roots. R1420 is also expressed in a seedling during the mature period more significantly than during the juvenile period. The predominant expression of genetic products in roots suggests that R1420 has a function contributing to characteristics of plants, such as an ability to grow under anaerobic conditions, such as under water and the like. The significant expression in a seedling during the mature period suggests that R1420 is required for the metabolism of a plant during the mature period, such as regulation of the amount of IAA which is a plant hormone, and the like. An exemplary primer having a sequence specific to this gene is R1420FP1 (SEQ ID NO: 48). A promoter derived from the R1420 gene is considered to reflect the expression specificity of R1420. Use of the R1420 gene and homologs thereof and promoters derived therefrom is useful for modification of plant characteristics.

R0317 is also a sequence obtained from rice based on the EST sequences, and belongs to A1 in accordance with the categorization described herein. This gene has a sequence indicated by SEQ ID NO: 7. R0317 may also be expressed predominantly in roots. R0317 is also expressed in a seedling during the mature period more significantly than during the juvenile period. The predominant expression of genetic products in roots suggests that R0317 has a function contributing to characteristics of plants, such as an ability to grow under anaerobic conditions, such as under water and the like. The significant expression in a seedling during the mature period suggests that R0317 is required for the metabolism of a plant during the mature period, such as regulation of the amount of IAA which is a plant hormone, and the like. An exemplary primer having a sequence specific to this gene is R0317F1 (SEQ ID NO: 47). A promoter derived from the R0317 gene is considered to reflect the expression specificity of R0317. Use of the R0317 gene and homologs thereof and promoters derived therefrom is useful for modification of plant characteristics.

S13316 is also a sequence obtained from rice based on the EST sequences, and belongs to A1 in accordance with the categorization described herein. This gene has a sequence indicated by SEQ ID NO: 9. S13316 may also be expressed predominantly in roots. S13316 is also expressed in a seedling during the mature period more significantly than during the juvenile period. The predominant expression of genetic products in roots suggests that S13316 has a function contributing to characteristics of plants, such as an ability to grow under anaerobic conditions, such as under water and the like. The significant expression in a seedling during the mature period suggests that S13316 is required for the metabolism of a plant during the mature period, such as regulation of the amount of IAA which is a plant hormone, and the like. An exemplary primer having a sequence specific to this gene is S13316FP1(SEQ ID NO: 54). A promoter derived from the S13316 gene is considered to reflect the expression specificity of S13316. Use of the S13316 gene and homologs thereof and promoters derived therefrom is useful for modification of plant characteristics.

R2151 is also a sequence obtained from rice based on the EST sequences, and belongs to Al in accordance with the categorization described herein. This gene has a sequence indicated by SEQ ID NO: 11. R2151 may also be expressed predominantly in roots. R2151 is also expressed in a seedling during the mature period more significantly than during the juvenile period. The significant expression in a seedling during the mature period suggests that R2151 is required for the metabolism of a plant during the mature period, such as regulation of the amount of IAA which is a plant hormone, and the like. This gene may exhibit reducible responses to cutting and rubbing stresses, and stimuli of wound information transfer substances (e.g., MeJA) and stimuli of ethylene release factors (e.g., ethephon). A promoter derived from the R2151 gene is considered to reflect the expression specificity of R2151. Use of the R2151 gene and homologs thereof and promoters derived therefrom is useful for modification of plant characteristics.

S4325 is also a sequence obtained from rice based on the EST sequences, and belongs to A1 in accordance with the categorization described herein. This gene has a sequence indicated by SEQ ID NO: 13. S4325 may also be expressed predominantly in roots. S4325 may be similarly expressed during the juvenile period and the mature period. The similar expression in a seedling during the juvenile period and the mature period suggests that S4325 is involved generally in the growth, elongation, and metabolism of plants. An exemplary primer having a sequence specific to this gene is S4325F1 (SEQ ID NO: 57). A promoter derived from the S4325 gene is considered to reflect the expression specificity of S4325. Use of the S4325 gene and homologs thereof and promoters derived therefrom is useful for modification of plant characteristics.

C62847 is also a sequence obtained from rice based on the EST sequences, and belongs to A1 in accordance with the categorization described herein. This gene has a sequence indicated by SEQ ID NO: 15. C62847 may also be similarly expressed in roots and aerial parts. C62847 may be similarly expressed during the juvenile period and the mature period. The similar expression in a seedling during the juvenile period and the mature period suggests that C62847 is involved generally in the growth, elongation, and metabolism of plants. The similar expression of genetic products in roots and aerial parts suggests that C62847 has a function contributing to characteristics of plants, such as an ability to grow under anaerobic conditions, such as under water and the like, and a function contributing to promotion and maintenance of the growth of aerial parts (e.g., the elongation of a stem and the like). An exemplary primer having a sequence specific to this gene is C62847FP1 (SEQ ID NO: 44). A promoter derived from the C62847 gene is considered to reflect the expression specificity of C62847. Use of the C62847 gene and homologs thereof and promoters derived therefrom is useful for modification of plant characteristics.

R1617 is also a sequence obtained from rice based on the EST sequences, and belongs to A1 in accordance with the categorization described herein. This gene has a sequence indicated by SEQ ID NO: 17. R1617 may also be similarly expressed in roots and aerial parts. R1617 may be similarly expressed during the juvenile period and the mature period. The similar expression in a seedling during the juvenile period and the mature period suggests that R1617 is involved generally in the growth, elongation, and metabolism of plants. The similar expression of genetic products in roots and aerial parts suggests that R1617 has a function contributing to characteristics of plants, such as an ability to grow under anaerobic conditions, such as under water and the like, and a function contributing to promotion and maintenance of the growth of aerial parts (e.g., the elongation of a stem and the like). A promoter derived from the R1617 gene is considered to reflect the expression specificity of R1617. Use of the R1617 gene and homologs thereof and promoters derived therefrom is useful for modification of plant characteristics.

R3025 is also a sequence obtained from rice based on the EST sequences, and belongs to A1 in accordance with the categorization described herein. This gene has a sequence indicated by SEQ ID NO: 19. Analysis of the putative amino acid sequence suggests that the protein product is of an extracellular secretory type. R3025 may also be similarly expressed in roots and aerial parts. R3025 may be similarly expressed during the juvenile period and the mature period. The similar expression in a seedling during the juvenile period and the mature period suggests that R3025 is involved generally in the growth, elongation, and metabolism of plants. The similar expression of genetic products in roots and aerial parts suggests that R3025 has a function contributing to characteristics of plants, such as an ability to grow under anaerobic conditions, such as under water and the like, and a function contributing to promotion and maintenance of the growth of aerial parts (e.g., the elongation of a stem and the like). A promoter derived from the R3025 gene is considered to reflect the expression specificity of R3025. Use of the R3025 gene and homologs thereof and promoters derived therefrom is useful for modification of plant characteristics.

R2391 is also a sequence obtained from rice based on the EST sequences, and belongs to A1 in accordance with the categorization described herein. This gene has a sequence indicated by SEQ ID NO: 21. R2391 may also be similarly expressed in roots and aerial parts. The similar expression of genetic products in roots and aerial parts suggests that R2391 has a function contributing to characteristics of plants, such as an ability to grow under anaerobic conditions, such as under water and the like, and a function contributing to promotion and maintenance of the growth of aerial parts (e.g., the elongation of a stem and the like). R2391 may be expressed during the juvenile period more significantly than during the mature period. The significant expression in a seedling during the juvenile period suggests that R2391 is involved in the growth and elongation of plants, such as synthesis of cell walls, and the like. An exemplary primer having a sequence specific to this gene is R2391FP2 (SEQ ID NO: 50). A promoter derived from the R2391 gene is considered to reflect the expression specificity of R2391. Use of the R2391 gene and homologs thereof and promoters derived therefrom is useful for modification of plant characteristics.

S10927 is also a sequence obtained from rice based on the EST sequences, and belongs to A2 in accordance with the categorization described herein. This gene has a sequence indicated by SEQ ID NO: 23. S10927 is expressed predominantly in aerial parts. The predominant expression of genetic products in aerial parts suggests that S10927 has a function contributing predominantly to promotion and maintenance of the growth of aerial parts (e.g., elongation of a stem, and the like). S10927 may also be similarly expressed during the juvenile period and the mature period. The similar expression in a seedling during the juvenile period and the mature period suggests that S10927 is required for the growth, elongation and metabolism of plants, such as involvement in synthesis of cell walls and regulation of the amount of a plant hormone IAA, and the like. An exemplary primer having a sequence specific to this gene is S10927FP1 (SEQ ID NO: 52). A promoter derived from the S10927 gene is considered to reflect the expression specificity of S10927. Use of the S10927 gene and homologs thereof and promoters derived therefrom is useful for modification of plant characteristics.

S14493 is also a sequence obtained from rice based on the EST sequences, and belongs to A2 in accordance with the categorization described herein. This gene has a sequence indicated by SEQ ID NO: 25. S14493 is expressed predominantly in aerial parts. The predominant expression of genetic products in aerial parts suggests that S14493 has a function contributing predominantly to promotion and maintenance of the growth of aerial parts (e.g., elongation of a stem, and the like). S14493 may also be similarly expressed during the juvenile period and the mature period. The similar expression in a seedling during the juvenile period and the mature period suggests that S14493 is involved generally in the growth, elongation and metabolism of plants. An exemplary primer having a sequence specific to this gene is S14493FP1 (SEQ ID NO: 56). A promoter derived from the S14493 gene is considered to reflect the expression specificity of S14493. Use of the S14493 gene and homologs thereof and promoters derived therefrom is useful for modification of plant characteristics.

prxRPA as well as the above-described prxPRN are POXs which have been isolated by the present inventors (Ito et al., (1994), Reference 12). The prxRPA gene has a gene sequence indicated by SEQ ID NO: 27 and is categorized as B1 in accordance with the categorization of the present invention. The genetic products of this gene are induced by various stresses. This suggests that prxRPA is involved in the defense mechanism of plants against stresses. Specifically, prxRPA may be induced by oxygen stresses (e.g., UV and paraquat), stimuli of wound information transfer substances (e.g., MeJA), stimuli of ethylene release factors (e.g., ethephon), and a rubbing stress (see Examples 3 to 5). Thus, it is suggested that this gene plays a role in defense against pathogens and removal of active oxygen species. prxRPA may also be significantly expressed in a seedling during the mature period. The significant expression during the mature period suggests that prxRPA is required for the metabolism of plants during the mature period, such as regulation of the amount of the plant hormone IAA, and the like. Analysis of the putative amino acid sequence suggests that the protein product is of an extracellular secretory type. Examples of a primer having a sequence specific to this gene include prxRPAFP1 (SEQ ID NO: 45) and prxRPARP1 (SEQ ID NO: 46). A promoter derived from the prxRPA gene is considered to reflect the expression specificity of prxRPA. Use of prxRPA gene and homologs thereof and promoters derived therefrom is useful for modification of plant characteristics.

R2576 is a sequence obtained from rice based on the EST sequences, and belongs to B1 in accordance with the categorization described herein. The genetic products of this gene are induced by various stresses. This suggests that R2576 is involved in the defense mechanism of plants against stresses. Specifically, the genetic products of R2576 may be induced by oxygen stresses (e.g., UV and paraquat), stimuli of wound information transfer substances (e.g., MeJA), stimuli of ethylene release factors (e.g., ethephon), and a rubbing stress (see Examples 3 to 5). Thus, it is suggested that this gene plays a role in defense against pathogens and removal of active oxygen species. R2576 may also be significantly expressed in a seedling during the mature period. The significant expression during the mature period suggests that R2576 is required for the metabolism of plants during the mature period, such as regulation of the amount of the plant hormone IAA, and the like. This gene has a gene sequence indicated by SEQ ID NO: 29. Analysis of the putative amino acid sequence suggests that the protein product is of an extracellular secretory type. An example of a primer having a sequence specific to this gene is R2576F1 (SEQ ID NO: 51). A promoter derived from the R2576 gene is considered to reflect the expression specificity of R2576. Use of R2576 gene and homologs thereof and promoters derived therefrom is useful for modification of plant characteristics.

R2184 is also a sequence obtained from rice based on the EST sequences, and belongs to B1 in accordance with the categorization described herein. The genetic products of this gene are induced by various stresses. This suggests that R2184 is involved in the defense mechanism of plants against stresses. Specifically, the genetic products of R2184 may be induced by oxygen stresses (e.g., UV), stimuli of wound information transfer substances (e.g., MeJA), and stimuli of ethylene release factors (e.g., ethephon) (see Examples 4 and 5). Also, such induction may be caused by stresses due to cutting into pieces (see Example 3). Therefore, it is considered that this POX is involved in defense, particularly against significant wounds occurring in plants. Thus, it is suggested that this gene plays a role in defense against pathogens and removal of active oxygen species. R2184 may also be significantly expressed in a seedling during the juvenile period. The significant expression during the juvenile period suggests that R2184 is required for the growth and elongation of plants, such as synthesis of cell walls, and the like. This gene has a gene sequence indicated by SEQ ID NO: 31. Analysis of the putative amino acid sequence suggests that the protein product is of a vacuole localization type. An example of a primer having a sequence specific to this gene is R2184FP1 (SEQ ID NO: 49). A promoter derived from the R2184 gene is considered to reflect the expression specificity of R2184. Use of R2184 gene and homologs thereof and promoters derived therefrom is useful for modification of plant characteristics.

R2693 is also a sequence obtained from rice based on the EST sequences, and belongs to B1 in accordance with the categorization described herein. The genetic products of this gene are induced by various stresses. This suggests that R2693 is involved in the defense mechanism of plants against stresses. Specifically, the genetic products of R2693 may be induced by oxygen stresses (e.g., UV), stimuli of wound information transfer substances (e.g., MeJA), and stimuli of ethylene release factors (e.g., ethephon) (see Examples 4 and 6). Also, such induction may be caused by stresses due to cutting into pieces (see Example 3). Therefore, it is considered that this POX is involved in defense particularly against significant wounds occurring in plants. Thus, it is suggested that this gene plays a role in defense against pathogens and removal of active oxygen species. R2693 may also be significantly expressed in a seedling during the juvenile period. The significant expression during the juvenile period suggests that R2693 is required for the growth and elongation of plants, such as synthesis of cell walls, and the like. This gene has a gene sequence indicated by SEQ ID NO: 33. Analysis of the putative amino acid sequence suggests that the protein product is of an extracellular secretory type. A promoter derived from the R2693 gene is considered to reflect the expression specificity of R2693. Use of R2693 gene and homologs thereof and promoters derived therefrom is useful for modification of plant characteristics.

C52903 is also a sequence obtained from rice based on the EST sequences, and belongs to B1 in accordance with the categorization described herein. The genetic products of this gene are induced by various stresses. This suggests that C52903 is involved in the defense mechanism of plants against stresses. Specifically, the genetic products of C52903 may be induced by oxygen stresses (e.g., UV and paraquat), stimuli of wound information transfer substances (e.g., MeJA), stimuli of ethylene release factors (e.g., ethephon), and rubbing and cutting stresses (see Examples 3 to 5). The inducibility of this POX by a wide range of stresses suggests that this gene plays a role in defense against various stresses. C52903 may also be significantly expressed in a seedling during the mature period. The significant expression during the mature period suggests that C52903 is required for the metabolism of plants during the mature period, such as regulation of the amount of the plant hormone IAA, and the like. This gene has a gene sequence indicated by SEQ ID NO: 35. Analysis of the putative amino acid sequence suggests that the protein product is of a vacuole localization type. An example of a primer having a sequence specific to this gene is C52903FP1 (SEQ ID NO: 43). A promoter derived from the C52903 gene is considered to reflect the expression specificity of C52903. Use of C52903 gene and homologs thereof and promoters derived therefrom is useful for modification of plant characteristics.

R2329 is also a sequence obtained from rice based on the EST sequences, and belongs to B2 in accordance with the categorization described herein. The genetic products of this gene are induced by various stresses. This suggests that R2329 is involved in the defense mechanism of plants against stresses. Specifically, the genetic products of R2329 may be induced by oxygen stresses (e.g., UV and paraquat), stimuli of wound information transfer substances (e.g., MeJA), stimuli of ethylene release factors (e.g., ethephon), and rubbing and cutting stresses (see Examples 3 to 5). The inducibility of this POX by a wide range of stresses suggests that this gene plays a role in defense against various stresses. R2329 may also be significantly expressed in a seedling during the mature period. The significant expression during the mature period suggests that R2329 is required for the metabolism of plants during the mature period, such as regulation of the amount of the plant hormone IAA, and the like. This gene has a gene sequence indicated by SEQ ID NO: 37. Analysis of the putative amino acid sequence suggests that the protein product is of an extracellular secretory type. A promoter derived from the R2329 gene is considered to reflect the expression specificity of R2329. Use of R2329 gene and homologs thereof and promoters derived therefrom is useful for modification of plant characteristics.

S11222 is also a sequence obtained from rice based on the EST sequences, and belongs to B2 in accordance with the categorization described herein. The genetic products of this gene are induced by various stresses. This suggests that S11222 is involved in the defense mechanism of plants against stresses. Specifically, the genetic products of S11222 maybe induced by stimuli of wound information transfer substances (e.g., MeJA), and stimuli of ethylene release factors (e.g., ethephon) (see Example 4). Thus, it is suggested that this gene plays a role in defense against pathogens. S11222 may also be significantly expressed in a seedling during the juvenile period. The significant expression during the juvenile period suggests that S11222 is required for the growth and elongation of plants, such as synthesis of cell walls, and the like. This gene has a gene sequence indicated by SEQ ID NO: 39. An example of a primer having a sequence specific to this gene is S11222FP1 (SEQ ID NO: 53). A promoter derived from the S11222 gene is considered to reflect the expression specificity of S11222. Use of S11222 gene and homologs thereof and promoters derived therefrom is useful for modification of plant characteristics.

S14082 is also a sequence obtained from rice based on the EST sequences. Herein, no expression of this gene was detected in experiments. Therefore, S14082 belongs to C in accordance with the categorization described herein. This gene has a gene sequence indicated by SEQ ID NO: 41. An example of a primer having a sequence specific to this gene is S14082FP1 (SEQ ID NO: 55). A promoter derived from the S14082 gene is considered to reflect the expression specificity of S14082. Use of S14082 gene and homologs thereof and promoters derived therefrom is useful for modification of plant characteristics.

(Method for Obtaining Each Gene and its Homologs and Method for Confirming the Expression Specificity)

The peroxidase (POX) genes of the present invention and homologs of the POX genes which hybridize to the POX genes under stringent conditions may be isolated using a degenerate primer pair corresponding to the non-conserved regions of the amino acid sequence encoded by the POX genes. This primer pair may be used and a cDNA or genomic DNA of any subject plant may be used as a template to carry out PCR. Thereafter, the resultant amplified DNA fragments may be used as a probe to screen a cDNA library or genomic library of the same subject plant. Next, positive clones are selected and subjected to sequencing, thereby characterizing the POX genes of the present invention or homologs thereof.

The thus-obtained POX genes of the present invention or homologs thereof may be confirmed to have a desired expression specificity by analyzing the expression characteristics of the original plant using the genes or fragments thereof as a selective probe. Alternatively, such a desired expression specificity may be confirmed by introducing the genes into any plant to produce a transformed plant in accordance with a method disclosed herein. RNA samples may be prepared from an appropriate plant material based on a desired expression characteristic, and subjected to northern blot analysis, thereby making it possible to confirm and compare expression amounts.

(Specification of Promoter)

It is well known that a promoter for each of the above-described genes can be obtained from the upstream sequence of the coding region. Such a promoter is representatively defined as, but is not limited to, a sequence present in the range of about 2 kb upstream of a translation initiation point.

A promoter region may be specified in accordance with a well-known method in the art. Briefly, a candidate sequence for a promoter region is operatively linked to a reporter gene (e.g., GUS gene) to construct an expression cassette. The constructed expression cassette is used to transform an appropriate plant cell. The transformed cell is regenerated to a plant. The expression of the reporter gene in the transformed plant is detected by utilizing an appropriate detection system (e.g., dye staining). Based on the results of the detection, the promoter region and its expression characteristics may be confirmed.

(Method for Modifying Plants by Utilizing Expression-Specific POX Genes)

As described above, the POX genes (structural gene) of the present invention and promoters therefor may be each useful as a material for modifying the characteristics of plants in a desired manner. Characteristics to be modified include, but are not limited to, resistance of plant to stresses, and characteristics relating to the growth or metabolism of plants (e.g., the rate or period of growth).

The POX gene of the present invention may be introduced into plant cells as an expression cassette in which each gene is operatively linked to an appropriate promoter. Further, the promoters of the present invention may be introduced into plant cells as an expression cassette in which each promoter is operatively linked to an appropriate heterologous gene, using a well-known method in the art. “Expression cassette” as used herein refers to a nucleic acid sequence containing DNA encoding a POX of the present invention and a plant gene promoter operatively linked thereto (i.e., the promoter can control the expression of the DNA), and a nucleic acid sequence containing a promoter of the present invention and a heterologous gene operatively linked thereto (i.e., linked in-frame thereto). Use of a naturally-occurring expression cassette containing a peroxidase gene optionally in combination with other regulatory elements falls within the scope of the present invention. A preferable expression cassette may be cut by a particular restriction enzyme and is easy to recover.

“Heterologous gene” which may be linked to the promoters of the present invention refers to any of the POX genes of the present invention other than POX genes from which the promoters are derived, plant endogenous genes other than the POX genes, or genes foreign to plants (e.g., genes derived from animals, insects, bacteria and fungi), provided that an expression cassette containing such a gene is introduced into a plant and the genetic products of the gene can be expressed in the plant.

“Plant gene promoter” which may be linked to the POX genes of the present invention means any promoters which can be expressed in plants. Examples of such a plant gene promoter include, but are not limited to, promoters, such as a tobacco PR1 a promoter and the like, of which the expression is induced by a certain stress, a CaMV35S promoter, a promoter (Pnos) for nopaline synthetase, and the like.

The above-described expression cassette is preferably utilized in the form of a plant expression vector. “Plant expression vector” refers to a nucleic acid sequence in which various regulatory elements as well as a structural gene and a promoter for regulating the expression are operatively linked in a host plant cell. Examples of the regulatory elements include, preferably, terminators, drug-resistant genes, and enhancers. It is well known to those skilled in the art that the types of plant expression vectors and the types of regulatory elements used may vary according to a host cell. The plant expression vectors used in the present invention may further have a T-DNA region. The T-DNA region can improve the efficiency of gene introduction when plants are transformed by, particularly, Agrobacterium.

“Terminator”is a sequence which is located downstream of a region encoding a protein of a gene and which is involved in the termination of transcription when DNA is transcribed into mRNA, and the addition of a polyA sequence. It is known that a terminator contributes to the stability of mRNA, and has an influence on the amount of gene expression. Examples of such a terminator include, but are not limited to, a CaMV35S terminator, a terminator for the nopaline synthetase gene (Tnos), and a terminator for the tobacco PR1a gene.

“Drug-resistant gene” refers to a gene which confers drug resistance to a host when the genetic product thereof is expressed in the host. The drug-resistant gene is desirably one that facilitates the selection of transformed plants. The neomycin phosphotransferase II (NPTII) gene for conferring kanamycin resistance, the hygromycin phosphotransferase gene for conferring hygromycin resistance, and the like may be preferably used.

“Enhancer” maybe used so as to enhance the expression efficiency of a gene of interest. As such an enhancer, an enhancer region containing an upstream sequence within the CaMV35S promoter is preferable. A plurality of enhancers may be used.

As a vector for use in construction of a plant expression vector, pBI vectors, pUC vectors, or pTRA vectors are preferably used. The pBI and pTRA vectors may be used to introduce a gene of interest into plants via Agrobacterium. pBI binary vectors or intermediate vectors may be preferably used. Examples of such vectors include pBI121, pBI101, pBI101.2, pBI101.3, and the like. These vectors contain a gene of a region (T-region) to be introduced into a plant, and the NPT2 gene (conferring kanamycin resistance) as a marker gene which is expressed under the control of a plant promoter. With pUC vectors, a gene may be introduced directly into plants. Examples of pUC vectors include pUC18, pUC19, pUC9, and the like. Plant expression vectors maybe produced using gene recombinant techniques well known to those skilled in the art.

For the purpose of introduction of a plant expression vector into a plant cell, a method well known to those skilled in the art, such as an indirect method using Agrobacterium, and a method for directly introducing into cells, can be used. As such an indirect method using Agrobacterium, for example, a method of Nagel et al. (Reference 19) may be used. In this method, initially Agrobacterium is transformed with a plant expression vector by electroporation, and then the transformed Agrobacterium is introduced into a plant cell with a method described in Gelvin et al. (Reference 20). As a method for directly introducing a plant expression vector into a cell, an electroporation method (see Shimamoto et al., Reference 21; and Rhodes et al., Reference 22), a particle gun method (see Reference 23), and a polyethylene glycol (PEG) method (see Reference 24) are illustrated. These methods are well known in the art. A method suitable for a plant to be transformed can be appropriately selected by those skilled in the art.

A cell into which a plant expression vector has been introduced is first selected according to drug resistance, such as kanamycin resistance, and the like. Thereafter, the cell may be regenerated to a plant tissue, a plant organ, and/or a plant using a well-known method in the art. Further, seeds may be obtained from the plant. The expression of introduced genes may be detected by a northern method or a PCR method. The expression of proteins which are genetic products may be confirmed by, for example, a western blot method.

The POX genes and promoters of the present invention may be utilized for modification of not only monocotyledons but also dicotyledons, this is because both have a similar genomic structure (Moore et al., Reference 25, and Nagamura et al., Reference 26). Particularly preferable examples of subject plants include wheat, maize, rice, barley, Sorghum, citrus, Chinese cabbage, lettuce, tobacco, peach, potato, tomato, apple, and the like. It has been demonstrated that the POX genes are capable of being introduced into plants, such as, Arabidopsis (Reference 27), Japanese aspen (Reference 28), sweet potato (Reference 29), rice (Reference 30), and the like.

A regenerated plant from a transformed cell may be confirmed to have a desired modified characteristic by carrying out an assay appropriate to the type of characteristic. For example, when it is intended to confer resistance to a pathogenic bacterium as stress resistance, a model bacterium (e.g., Pseudomonas syringae pv. tabaci) is inoculated into a regenerated plant which is in turn compared with a control plant so as to observe the presence or absence of a change due to the inoculation. As a result, a change in the characteristic can be evaluated.

Alternatively, the stress resistance of transformed plants may be evaluated as resistance to an UV treatment, resistance to a treatment with a superoxide generation type herbicide (e.g., paraquat), and/or resistance to salt stress, and the like.

(Gene Expression Assay)

The present invention also provides the peroxidase genes of the present invention or a set thereof, or a method for analyzing the characteristics of plants using an oligonucleotide containing a sequence derived from a promoter. Examples of such a method include northern blot analysis and the like. Such a method is reviewed in, for example, Sambrook J. et al. (Reference 60), Reference 36, and the like.

(DNA Array)

The nucleotides of the present invention may be used in a gene analysis method using a DNA array. A DNA array is widely reviewed (Shujunsha Ed., Saibo-kogaku (Cellular Engineering), Special issue, “DNA-maikuro-arei-to-saisin-PCR-ho [DNA microarray and Up-to-date PCR Method”). Further, plant analysis using a DNA array has been recently used (Reference 58). Hereinafter, a DNA array and a gene analysis method using the same will be briefly described.

“DNA array” refers to a device in which DNAs are arrayed and immobilized on a plate. DNA arrays are divided into DNA macroarrays, DNA microarrays and the like according to the size of a plate or the density of DNA placed on the plate.

The border between macro and micro is not strictly determined. However, generally, “DNA macroarray” refers to a high density filter in which DNA is spotted on a membrane, while “DNA microarray” refers to a plate of glass, silicon, and the like which carries DNA on a surface thereof. There are a cDNA array, an oligoDNA array, and the like according to the type of DNA placed.

A certain high density oligoDNA array, in which a photolithography technique for production of semiconductor integrated circuits is applied and a plurality of oligoDNAs are simultaneously synthesized on a plate, is particularly called “DNA chip”, an adaptation of the term “semiconductor chip”. Examples of the DNA chip prepared by this method include GeneChip® (Affymetrix, CA), and the like (References 50 and 51). Preferably, GeneChip® may be used in gene analysis using a microarray according to the present invention. The DNA chip is defined as described above in narrow sense, but may refer to all types of DNA arrays or DNA microarrays.

Thus, DNA microarrays are a device in which several thousands to several ten thousands or more of gene DNAs are arrayed on a glass plate in high density. Therefore, it is made possible to analyze gene expression profiles or gene polymorphism at a genomic scale by hybridization of cDNA, cRNA or genomic DNA. With this technique, it has been made possible to analyze a signal transfer system and/or a transcription control pathway (Reference 45); the mechanism of tissue repair (Reference 46); the action mechanism of medicaments (Reference 47); fluctuations in gene expression during development and differentiation processes in a wide scale, and the like; identify a gene group whose expression is fluctuated according to pathologic conditions; find a novel gene involved in a signal transfer system or a transcription control; and the like. Further, as to gene polymorphism, it has been made possible to analyze a number of SNP with a single DNA microarray (Reference 48).

(Principle of Assay Using DNA Microarray)

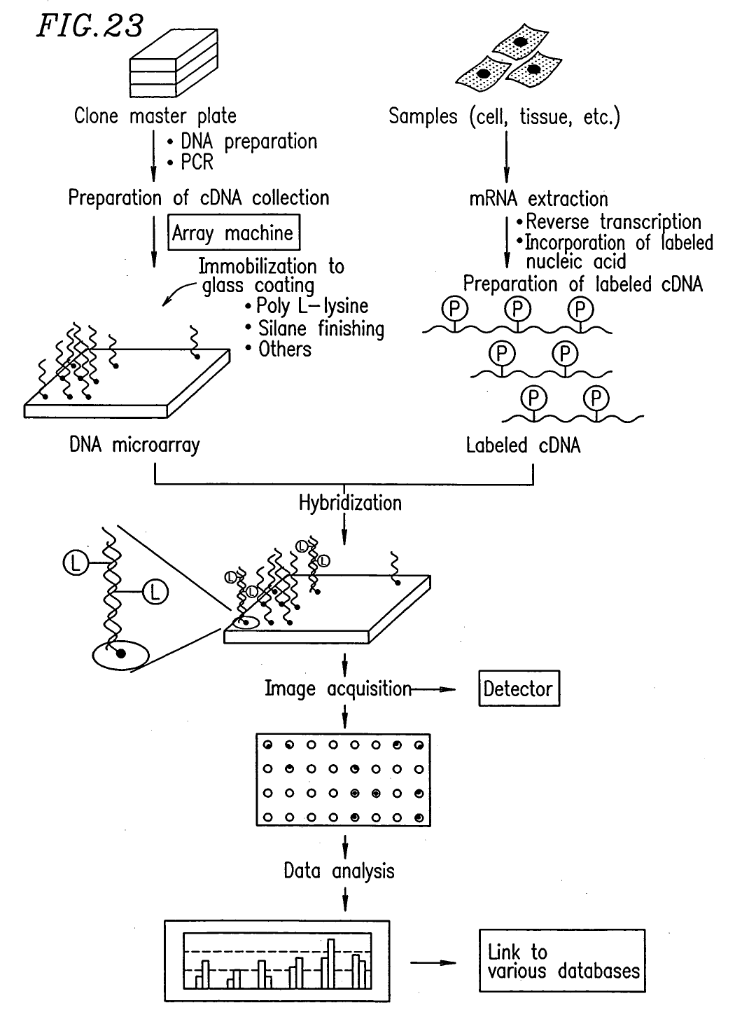

The principle of assay using a DNA microarray will be described. DNA microarrays are prepared by immobilizing a number of different DNA probes in high density on a solid-phase plate, such as a slide glass, whose surface is appropriately processed. Thereafter, labeled nucleic acids (targets) are subjected to hybridization under appropriate hybridization conditions, and a signal from each probe is detected by an automated detector. The resultant data is subjected to massive analysis by a computer. For example, in the case of gene monitoring, target cDNAs integrated with fluorescent labels by reverse transcription from mRNA are allowed to hybridize to oligo DNAs or cDNAs as a probe on a microarray, and are detected with a fluorescence image analyzer. In this case, T7 polymerase may be used to carry out other various signal amplification reactions, such as cRNA synthesis reactions or via enzymatic reactions. A flow of a DNA microarray experiment is shown in FIG. 23.

(Synthesized DNA Chip)

Fodor et al. has developed a technique for synthesizing polymers on a plate using a combination of combinatorial chemistry and photolithography for semiconductor production (Reference 53). This is called the synthesized DNA chip. Photolithography allows extremely minute surface processing, thereby making it possible to produce a DNA microarray having a packing density of as high as 10 μm²/DNA sample. In this method, generally, about 25 to about 30 DNAs are synthesized on a glass plate.

Gene expression using a synthesized DNA chip was reported by Lockart et al. (Reference 54). This method overcomes a drawback of the chip of this type in that the specificity is low since the length of synthesized DNA is short. This problem was solved by preparing perfect match (PM) oligonucleotide probes corresponding to from about 10 to about 20 regions and mismatch (MM) oligonucleotide probes having one base mutagenesis in the middle of the PM probes for the purpose of monitoring the expression of one gene. Here, the MM probes are used as an indicator for the specificity of hybridization. Based on the signal ratio between the PM probe and the MM probe, the level of gene expression may be determined. When the signal ratio between the PM probe and the MM probe is substantially 1:1, the result is called cross hybridization, which is not interpreted as a significant signal.

(Attached DNA Microarray)

A so-called attached DNA microarray is prepared by attaching DNAs onto a slide glass, and fluorescence is detected (54) (see also http://cmgm.stanford.edu/pbrown). In this method, no gigantic semiconductor production machine is required, and only a DNA array machine and a detector can be used to perform the assay in a laboratory. This method has the advantage that it is possible to select DNAs to be attached. A high density array can be obtained by spotting spots having a diameter of 100 μm at intervals of 100 μm, for example. It is mathematically possible to spot 2500 DNAs per cm². Therefore, a usual slide glass (the effective area is about 4 cm²) can carry about 10,000 DNAs.

As a labeling method for synthesized DNA arrays, for example, double fluorescence labeling is used. In this method, two different mRNA samples are labeled by respective different fluorescent dyes. The two samples are subjected to competitive hybridization on the same microarray, and both fluorescences are measured. By comparing the fluorescences, a difference in gene expression is detected. Examples of the fluorescent dye include, but are not limited to, Cy5 and Cy3, which are most often used, and the like. The advantage of Cy3 and Cy5 is that the wavelengths of fluorescences do not overlap substantially. Double fluorescence labeling may be used to detect mutations or morphorisms in addition to differences in gene expression.

An array machine may be used for assay using a DNA array. In the array machine, basically, a pin tip or a slide holder is moved in directions along X, Y and Z axes in combination with a high-performance servo motor under the control of a computer so that DNA samples are transferred from a microtiter plate to the surface of a slide glass. The pin tip is processed into various shapes. For example, a DNA solution is retained in a cloven pen tip like a crow's bill and spotted onto a plurality of slide glasses. After washing and drying cycles, a DNA sample is then placed on the slide glasses. The above-described steps are repeated. In this case, in order to prevent contamination of the pin tip by a different sample, the pin tip has to be perfectly washed and dried. Examples of such an array machine include SPBIO2000 (Hitachi Software Engineering Co., Ltd.; single strike type), GMS417 Arrayer (Takara Shuzo Co., Ltd.; pin ring type), Gene Tip Stamping (Nippon Laser&Electronics Lab.; fountain pen type), and the like.

There are various DNA immobilizing methods for use in assays using a DNA array. Glass as a material for a plate has a small effective area for immobilization and electrical charge amount as compared to membranes, and therefore is given various coatings. In practice, Poly L-lysine coating (Reference 55), silane finishing (Reference 56), or the like. Further, a commercially available precoated slide glass exclusive to DNA microarrays (e.g., polycarboimide glass (Nissin Spinning Co., Ltd.) and the like) may also be used. In the case of oligoDNA, a method of aminating a terminal of the DNA and crosslinking the DNA to silane-finished glass is available.

(Method for Preparing cDNA Collection)

DNA microarrays may carry mainly cDNA fragments amplified by PCR. When the concentration of cDNA is insufficient, signals cannot be sufficiently detected in some cases. In such a case when a sufficient amount of cDNA fragments is not obtained by one PCR operation, PCR is repeated some times. The resultant overall PCR products may be purified and condensed at one time. A probe cDNA may generally carry a number of random cDNAs, but may carry a group of selected genes (e.g., the gene or promoter groups of the present invention) or candidate genes for gene expression changes obtained by RDA (representational differential analysis) according to the purpose of an experiment. It is preferable to avoid overlapping clones. Clones may be prepared from a stock cDNA library, or cDNA clones may be purchased.

In assays using a DNA array, a fluorescent signal indicating hybridization on the DNA microarray is detected by a fluorescence detector or the like. As such a detector, there are conventionally various available detectors. For example, a research group at the Stanford University has developed an original scanner which is a combination of a fluorescence microscope and a movable stage (see http://cmgm.stanford.edu/pbrown). A conventional fluorescence image analyzer for gel, such as FMBIO (Hitachi Software Engineering), Storm (Molecular Dynamics), and the like, can read a DNA microarray if the spots are not arrayed in very high density. Examples of other available detectors include ScanArray 4000 and 5000 (GeneralScanning; scan type (confocal type)), GMS418 Array Scanner (Takara Shuzo; scan type (confocal type)), Gene Tip Scanner (Nippon Laser&Electronics Lab.; scan type (non-confocal type)), Gene Tac 2000 (Genomic Solutions; CCD camera type)), and the like.

(Data Analysis Software)

The amount of data obtained from DNA microarrays is huge. Software for managing correspondences between clones and spots, analyzing data, and the like is important. As such software, software attached to each detection system is available (57). Further, an example of a database format is GATC (genetic analysis technology consortium) proposed by Affymetrix.

(Differential Display Technique)

The present invention may also be used in gene analysis using a differential display technique.

The differential display technique is a method for detecting or identifying a gene whose expression fluctuates. In this method, cDNA is prepared from each of at least two samples, and amplified by PCR using a set of any primers. Thereafter, a plurality of generated PCR products are separated by gel electrophoresis. After the electrophoresis pattern is produced, expression fluctuating genes are cloned based on a relative signal strength change between each band.

The genes, gene groups, methods for using the same, and methods for analyzing the same using DNA arrays of the present invention are described above in detail. Thereinafter, examples of the present invention will be described.

### EXAMPLES

Examples described below illustrate the present invention, but not limit the present invention in any manner. Therefore, those skilled in the art may modify the present invention based on the matters described in Examples without departing from the scope of the claims.

It should be noted that reagents used in the following examples were obtained from Wako Pure Chemical Industries, Ltd. unless otherwise specified.

### Example 1

### Selection of POX Genes, Sequencing and Phylogenetic Analysis

Materials shown in Table 2 below were each used to extract mRNA by a commonly used method. cDNA was synthesized from the mRNA using reverse transcriptase. The cDNA was inserted into a plasmid vector pBluescript SK(+) (Stratagene) in a predetermined direction. This plasmid vector was used to transform a host E. coli strain NM522. The resultant transformed clones were picked up at random, and stored at −80° C. In order to estimate the genetic products of the obtained cDNA clones, a partial base sequence of each clone was determined from the 5′ end using ABI 373A DNA sequencing machine (PE Biosystems). The determined sequences had an average length of about 300 bp. From the resultant sequences, amino acid sequences were estimated using three reading frames. The resultant amino acid sequences were subjected to a similarity search in the NBRF-PIR database using the FASTA algorithm. As a result of the search, when the similarity score relative to a peroxidase protein, which indicated the highest similarity, was greater than or equal to 200, the clone having the score was regarded as having an amino acid sequence derived from rice which has a significant level of homology to peroxidase.

TABLE 2

| Number | Library Name | Organ | Vector |
|---|---|---|---|
| C1 | BA Callus | Callus from scutellum (BA treatment) | pBluescriptSK(+) |
| C2 | C(−) Callus | Callus from scutellum(C deletion) | pBluescriptSK(−)(λUni-ZAP XR) |
| C3 | NAA Callus | Callus from scutellum(NAA and BA treatments) | pBluescriptSK(−)(λUni-ZAP XR) |
| C4 | Root Callus | Callus from root tip | pBluescriptSK(−)(λUni-ZAP XR) |
| C5 | GA3 treatment | Callus from scutellum (GA treatment) | pBluescriptSK(+) |
| | Callus | | |
| C6 | Heat shock Callus | Callus from scutellum(heat shock treatment) | pBluescriptSK(+) |
| C | Growth Phase Callus | Callus from scutellum (growth phase) | pBluescriptSK(+) |
| (V) | Developing Seed | Developing seed (5DAF to 15DAF) | pBluescriptSK(+) |
| E | Flowering Panicle | Flowering panicle | pBluescriptSK(+) |
| E1 | Ripening Panicle | Ripening panicle | pBluescriptSK(+) |
| E2 | Young panicle 3 | Panicle (longer than 10 cm) | pBluescriptSK(+) |
| E3 | Young panicle 1 | Panicle (less than 3 cm) | pBluescriptSK(+) |
| E4 | Young panicle 2 | Panicle (3-10 cm) | pBluescriptSK(+) |
| E5 | Apical | Immature leaf containing apical meristem | pBluescriptSK(+) |
| | meristem(long) | (long day condition) | |
| E6 | Apical | Immature leaf containing apical meristem | pBluescriptSK(+) |
| | meristem(short) | (short day condition) | |
| F1 | Early heading lines | Leaf (light period insensitive. one day | λZAP II |
| | | treatment of short day) | |
| F | Late heading lines | Leaf (light period sensitive, one day | λZAP II |
| | | treatment of short day) | |
| R | Root | Root of shoot | pBluescriptSK(+) |
| | Chilled Root | Root of shoot | pBluescriptSK(+) |
| R1 | Adult leaf (SD) | Mature leaf (short day condition) | pBluescriptSK(+) |
| S2 | Shoot | Green shoot | 1-9 6; pBluescriptSK(−)(λUni-ZAP XR), 101-; |
| | | | pBluescriptSK(+) |
| S | Etiolated Shoot | Etiolated shoot | 1-9 6: pBluescriPtSK(−)(λUni-ZAP XR). 101-; |
| | | | pBluescriptSK(+) |

A part of the gene sequences having homology to peroxidase are registered as EST sequences and available from a public databank (DDBJ (DNA Data Bank of Japan)). Its URL is http://www.ddbj.nig.ac.jp/).

In an example below, cDNAs isolated from cDNA libraries derived from gibberellin (GA₃) callus, heat shock callus, roots, green shoots, and etiolated shoots were used. Out of the clones of the present invention, the clones whose initial letter is R are derived from library R (root), the clones having 4 digits following S are derived from library S (etiolated shoots), the clones having 4 digits following S1 are derived from library S1 (shoot), the clones having 4 digits following C5 are derived from library C5 (GA₃ treatment callus), and the clones having 4 digits following C6 are derived from library C6 (heat shock callus).

34 of the thus-obtained cDNAs having a putative open reading frame of peroxidase were selected and sequenced from both sides.

31 of the sequenced cDNAs were selected, and 5 cDNAs isolated in Ito et al. (1994) (Reference 12) and Chittoor et al. (1997) (Reference 10) were selected. For a total of 36 cDNAs, their putative amino acid sequences were analyzed by Kimura's protein distance prediction method (Kimura et al., Reference 31) and a neighbor joining method (Saitou et al., Reference 32, and Studier et al., Reference 33) to construct a phylogenetic tree. Analysis of amino acid sequences was limited to a region between upstream of the proximal heme binding domain and the vicinity of the 5^{th} cysteine invariable residue (this portion corresponds to a region of glycine 168 to proline 210 of horseradish POX C isozymes (Welinder et al., Reference 34)). This is because this region has been proposed to be important for catalytic activity determining the physiological function of each POX (Chittoor et al. (1997), Reference 10). EST clones (the remaining 3 out of 34) lacking histidine residue 2 and cysteine residue 8 which characterize POX were excluded from the phylogenetic analysis.

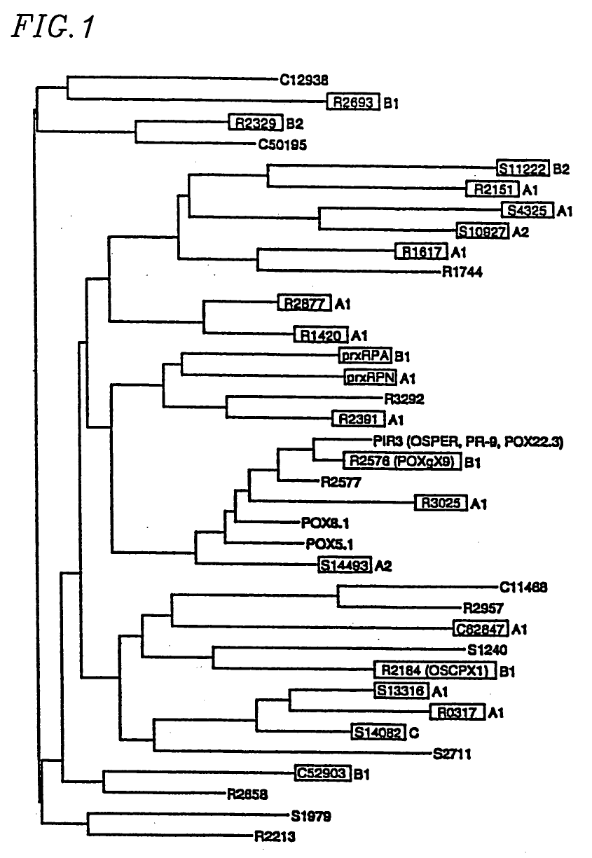

Thus, the 36 rice POXs were subjected to the phylogenetic analysis based on their amino acid sequences. The result is shown in FIG. 1.

The above-described 36 rice POXs are divided into a plurality of clusters. Seven POXs (PIR3, R2576, R2577, R3025, POX8.1, POX5.1 and S14493) are grouped into the same cluster, and exhibit a high level of homology to pathogen-induced POXs which have been found in a plurality of plants (Chittoor (1999), Reference 6). In fact, it has been observed that POX22.3 (identical to PIR3) and POX8.1 are induced in rice leaves infected with Xanthomonas oryzae pv. oryzae (Chittoor et al., (1997), Reference 10). Similarly, the POX gene of the other clusters shown in FIG. 1 are considered to have a common or related role according to their proximity on the phylogenetic tree. Representative POX genes were selected evenly from these clusters, and used in the examples below. The names of the selected genes are indicated by boxes in FIG. 1.

### Example 2

### Changes in Expression of Rice Peroxidases Derived from Growth Stages and Plant Portions

(Growth of Rice)

Rice (Oryza sativa cv. Nipponbare) was cultivated at a green house (20° C. to 32° C.). Roots and aerial parts of the 5-day-old young seedlings were obtained, and roots, leaf sheaths and leaf blades of the 16-day-old mature seedlings were obtained. These materials were used in the experiments below.

(Analysis of Gene Expression)

Gene expression was analyzed by subjecting total RNAs isolated by the ATA method (Nagy et al., Reference 35) to RNA gel blot analysis (Ausubel et al., Reference 36).

cDNA fragments containing 3′ untranslated regions of 21 rice POX genes indicated by boxes in FIG. 1 were amplified by the PCR method. The amplified fragments were used as probes specific to the respective POX genes for the RNA gel blot analysis. Major sequences (SEQ ID NO: 43 to 59) used in the production of each clone-specific probe are shown in Table 3.

TABLE 3

PCR probe specific to each POX

| | | | | |
|---|---|---|---|---|
| | SEQ | | | |
| | ID | | Length | Tm |
| Primer | NO. | Sequence (5′-3′) | (bp) | (° C.) |

| | | | | |
|---|---|---|---|---|
| C52903FP1 | 43 | TGTGCCCGTCGAACGCGTCG | 20 | 63 |
| C62847FP1 | 44 | TGCCCGCTCAGCTACAGC | 18 | 60 |
| prxRPAFP1 | 45 | AACCTCCAGAGCCTCTGTGC | 20 | 59 |
| prxRPARP1 | 46 | AAGGCACATACATTCAGTTC | 20 | 51 |
| R0317F1 | 47 | TCAAGACGTTCGACCTGG | 18 | 55 |
| R1420FP1 | 48 | TTCACCTCTGACGCGGCG | 18 | 60 |
| R2184FP1 | 49 | CGACAACAAGTACTACTTCG | 20 | 58 |
| R2391FP2 | 50 | GCCTCTACAACGAGACGG | 18 | 58 |
| R2576F1 | 51 | TCAAGGCCAACTGCCCA | 17 | 55 |
| S10927FP1 | 52 | CGACCTCGCCGCGCTGTCCG | 20 | 67 |
| S11222FP1 | 53 | GACGACGGCGCCCATCGTCG | 20 | 65 |
| S13316FP1 | 54 | GCGACAACACGACGCTGGCG | 20 | 63 |
| S14082FP1 | 55 | TCTTCCACTCCGACTCCGCG | 20 | 61 |
| S14493FP1 | 56 | CGGCGGCGACACCAACCTGG | 20 | 65 |
| S4325F1 | 57 | ATGTTCAGCGCCAAGGGC | 18 | 57 |
| prxRPNFP1 | 58 | CCTCGTCTCCAGCTCCGGCG | 20 | 65 |
| prxRPNRP1 | 59 | TTAAACCATATGGCAGTTGC | 20 | 51 |

The above-described RNAs were subjected to electrophoresis, followed by transcription to a membrane (HyBond N, Amersham). The specific probes were allowed to hybridize, followed by washing for 5 min (once) and for 10 min (twice) in 2×SSC containing 0.1% SDS at room temperature, and then three times in 1×SSC containing 0.1% SDS for 15 min each at 65° C. Subsequently, the membrane was subjected to autoradiography at −80° C. using a film for autoradiography (XA OMT, Kodak) overnight or more. The recovered film was analyzed by the BAS2000 Bioimaging analyzer (Fuji Photo Film Co., Ltd.) or Phosphor Imager SI (Molecular Dynamics) in accordance with the manufacturer's instruction. The amount of RNA loaded was confirmed by monitoring the level of ribosome RNA (rRNA) stained by methylene blue. The expression amounts of the POXs were compared with each other with reference to the rRNA levels.

(Changes in Expression Amount According to Growth Stage and Plant Site)

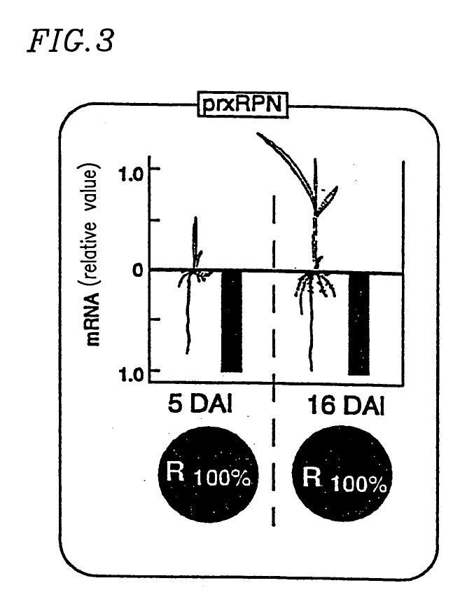

5-day-old and 16-day-old rice plant seedlings were produced in a manner as described above. Total RNAs from each sample were analyzed in a manner as described above. The expression amounts of the POXs were compared between different growth stages and between the roots and the aerial parts. The comparison results are shown in FIGS. 2A to 2C. The expression patterns of the POX genes are arranged in FIGS. 3 to 22. As can be seen from the figures, the 21 POX genes analyzed exhibited various expression patterns according to the growth stage and the plant site. Except for S14802, all POX genes analyzed expressed mRNA in the roots of both 5- and 16-day-old seedlings. Transcripts for 17 POX genes were detected in aerial parts (including leaf sheaths and leaf blades).

The number of types of POXs expressed on day 16 was reduced as compared to that of the 5-day-old seedlings. The reason is considered to be that POXs particularly required in the growth stage are expressed only in the juvenile period and are no longer used after the growth.

According to the results of this example, the 21 POX genes analyzed are divided into 5 classes, A1, A2, B1, B2 and C. These classes are described next to the boxes in FIG. 1. Site specificity where the expression ratio of root/aerial part is at least about 4/6 is categorized as “root≧aerial part”, while site specificity where the ratio is less than that value is categorized as “root<aerial part”.

Nine POX genes belonging to group Al other than R2151 and R3025 were not detected in the leaf blades of 16-day-old plants. For R2151 and R3025, considerable amounts of transcript were detected in the leaf blades of the 16-day-old plants. This suggests involvement of group A1 POX genes in the basic metabolism of plant growth (e.g., crosslinking of cell wall proteins and feruloylated polysaccharides, lignification, suberization and auxin degradation). R2151, S4325, R2877, R1420, prxPRN, S13316 and R3017 were predominantly expressed in roots, while R1617, R2391, R3025 and C62847 were similarly expressed in roots and aerial parts. Therefore, these genes are categorized as group A1. The site specificity indicates that each POX gene has a role at a site at which the gene is expressed. For S10927 and S14493, their expression levels were higher in aerial parts than in roots. Therefore, these genes are categorized as group A2. It is suggested that these POX genes play a basic role in aerial parts. Further, as described in examples below, POX genes belonging to groups A1 and A2 did not respond to stressestimuli, indicating that the genes play a basic role which is not inhibited by environmental stresses.

R2693, prxPRA, R2576, R2184 and C52903 genes were predominantly expressed in roots. Therefore, these genes are categorized as group B1. R2329 and S11222 were predominantly expressed in aerial parts. Therefore, these genes are categorized as group B2. These group B genes responded to stresses as indicated in examples below.

### Example 3

### Inducibility of Rice Peroxidases to Physical Stressestimuli

16-day-old seedlings were produced under the conditions as described in Example 2. The seedlings were used to analyze the inducibility of rice peroxidase to a cutting stress and a rubbing stress as a physical stress. A cutting stress was given by cutting the tips of the leaf blades by commercially available pruning shears. A rubbing stress was given by rubbing the whole blades by hands using caborundum #600 (Nacalai Tesque).

RNAs were extracted from the leaf sheaths of a rice plant given stimuli in a manner as described in Example 2. For each POX, expression specificity was analyzed. As a control, leaf blades which were not given a stress were used. The analysis results are shown in FIGS. 2A to 2C, and the results from the respective genes are shown in FIGS. 3 to 22. Plants or leaf blade sections treated were incubated under continuous irradiation (200 μE/m²/s) at 25° C. for 48 hours, followed by sampling.

For C52903 belonging to B1 and R2329 belonging to B2, their gene expressions were responsive to both a cutting stress and a rubbing stress. For prxRPA and R2576 genes belonging to B1, their expressions were induced only by a rubbing stress. For R2693 and R2184 belonging to B1, although expression was induced when leaf blades were cut into small pieces (no data shown), expression was not induced by the cutting stress or rubbing stress which was carried out in this example. This suggests that like cutting stresses lead to different expression-induction specificities according to the treatment methods. Both cutting stresses and rubbing stresses cause wound stresses. It is known that these wound stresses inhibit the normal growth and regeneration of plants, so that pathogens can easily invade plant tissues. Therefore, it is suggested that expression of peroxidase genes are controlled specifically in various stages such as suberization, lignification, and crosslinking of cell wall proteins, and the like in the course of restoration from wound stresses.

POX genes inducible to a certain wound stress may be induced by pathogens (Chittoor (1997), Reference 10, and Mohan et al., Reference 37). Therefore, it is suggested that wound stress-inducible POXs shown in this example are also involved in a defense system against pathogen infection.

### Example 4

### Inducibility of Rice Peroxidases to Ethephon and MeJA

16-day-old seedlings were produced under the conditions described in Example 2. With these seedlings, the inducibility of rice peroxidases to ethephon (an ethylene release factor) and MeJA (a wound information transfer substance) was analyzed. For ethephon stimulus, 1 mM ethephon solution containing 0.05% ethanol was used. For MeJA stimulus, 25 μM MeJA solution containing 0.125% Triton X-100 was used. These solutions were sprayed onto whole plants, and maintained for 48 hours.

RNAs were extracted from the leaf blades of stimulated rice plants in a manner as described in Example 2. For each POX, expression specificity was analyzed. As a control, leaf blade without a stress were used. The analysis results are shown in FIGS. 2A to 2C, and the results from the respective genes are shown in FIGS. 3 to 22.

Ethephon is known as an ethylene release factor. MeJA is known to function as a wound information transfer substance and the like. These factors induced expression of R2693, R2329, S11222, prxRPA, R2576, R2184 and C52903 POX genes. These genes were induced in a manner similar to wound stresses, drug stresses and a UV stress in Examples 3 and 5. Therefore, it is suggested that jasmine acid (JA) and ethylene are signal compounds for the stress-inducible expression of rice POX genes. In fact, JA accumulates locally or systemically in wounded rice plants (Schweizer et al., Reference 38, and Schweizer et al., Reference 39). Therefore, these 7 MeJA-inducible POX genes are suggested to be pathogen-inducible.

### Example 5

### Inducibility of Rice Peroxidases to Oxidative Stresses

16-day-old seedlings were produced under the conditions described in Example 2. These samples were used to analyze the inducibility of rice peroxidases to ultraviolet light stimuli and paraquat as oxidative stresses. For paraquat, 1 μM paraquat solution was used. To give paraquat stimuli, leaf blade sections were suspended in the 1 μM solution for 48 hours. Ultraviolet light stimuli were given by subjecting leaf blade sections suspended in sterilized water to ultraviolet light at 175 μW/cm² for 7 minutes. As a light source for ultraviolet light, a sterilization lamp (GL-15, NEC) was used.

RNAs were extracted from the leaf blades of stimulated rice plants in a manner as described in Example 2. For each POX, expression specificity was analyzed. As a control, leaf blade without a stress were used. The analysis results are shown in FIGS. 2A to 2C, and the results from the respective genes are shown in FIGS. 3 to 22.

As can be seen from FIGS. 2A to 2C, expression was induced by paraquat for R2329, prxRPA, R2576 and C52903. Further, for R2693, R2329, prxRPA, R2576, R2184 and C52903, expression was induced by ultraviolet light irradiation. These are peroxidases in the same group as those whose expression was induced by wound stresses. Moreover, the expression pattern caused by paraquat treatments was similar to that caused by wound stress.

Paraquat is a nonselective contact herbicide. Paraquat inhibits proton translocation through a thylakoid membrane, leading to generation of active oxygen species and energy depletion (Babbs et al., Reference 40). It has been reported that ultraviolet light causes H₂O₂ accumulation (Murphy et al., Reference 41). Among various types of peroxidases, it had been believed that ascorbic acid peroxidase is the only H₂O₂ scavenging enzyme in plants (Asada, Reference 42). Recent progresses of biochemical research suggests that class III peroxidases including the peroxidases of the present invention are also involved in scavenging of H₂O₂ (Mehlhorn et al., Reference 43, and Kvaratskhelia et al., Reference 44). It is suggested that the peroxidases which are induced by paraquat as indicated in this example are involved in detoxification of H₂O₂ and the like generated by active oxygen species.

### Example 6

### Analysis of RNA Expression Pattern in Plants Inoculated with Rice Blast Fungus (Magnaporthe grisea) and POX Genes as Marker Gene

Next, the relationship between the POX gene groups of the present invention and rice blast was investigated. As experimental strains, three rices, i.e., rice blast-susceptible rice (Oryza sativa cv. Nipponbare) (hereinafter referred to as compatible or −Pi-i in this example), rice blast-resistant rice (Oryza sativa cv. Nipponbare) (referred to as incompatible or +Pi-i in this example; available from the Nat. Agr. Res. Cent. of Japan); see Reference 61), and a compatible rice treated with probenazole (available as Oryzemate granule from Meiji Seika Kaisha Ltd.) (100 mg/ml) which is a rice blast control agricultural chemical, at the 8 leaf stage (6 weeks old, the height was almost 40 cm) were used. Each plant was cultivated in a green house (20° C. to 32° C.).

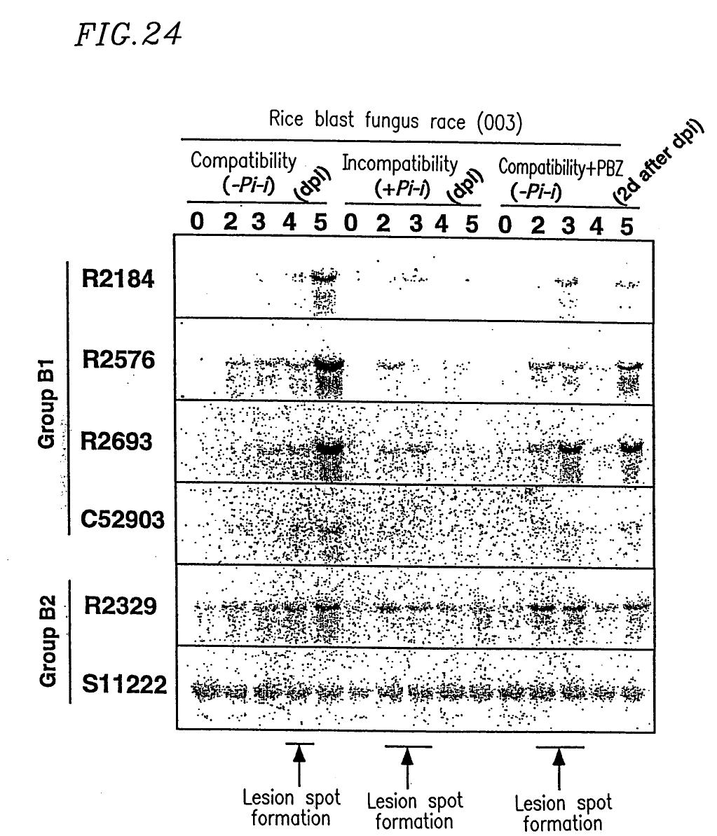

Each rice plant strain was treated with rice blast fungus (M. grisea race(003)) (1×10⁵ spores/ml). The time point of the treatment is regarded as day 0 (a probenazole treated group was treated with rice blast fungus two days after the probenazole treatment). Total RNAs immediately before the treatment and 2, 3, 4 and 5 days after the treatment were prepared in a manner as described above, and were subjected to northern analysis using the POX genes of the present invention. In this experiment, R2184, R2576, R2693 and C52903 (group B1), R2329 and S11222 (group B2), R3025 (group A1), and prxRPA (group B1) were used. The results are shown in FIG. 24.

Next, formation of lesion spots caused by rice blast fungus was observed. For the compatible rice, lesion spots were found on days 4 and 5. Thereafter, formation of rice blast fungus spores was observed. For the incompatible rice, formation of lesion spots was found on days 2 to 3. These lesion spots have a particular form for enclosing rice blast fungus. Thereafter, formation of rice blast fungus spores was not found. For the probenazole-treated rice, lesion spot formation was found on days 2 to 3, as for the incompatible rice, and the lesion spots were similar to that of the compatible rice.

For R3025 and prxRPA, no significant signal was found (no data shown). Further, as can be seen from FIG. 24, it is found that expression patterns of the compatible rice and the probenazole-treated rice when E2184, R2576, R2693 and R2329 were used as probes were significantly similar to the transition pattern of formation of lesion spots. These patterns match the transition pattern of lesion spot formation when another marker gene was used (no data shown).

Therefore, it was demonstrated that the POX genes of the present invention or gene groups thereof can be utilized as a marker for responses to pathogenic bacteria, such as rice blast fungus and the like, as an example of stress responses.

### Example 7

### Gene Expression Analysis on DNA Microarray

Next, gene expression was analyzed using DNA microarrays where the POX gene groups were used as markers.

As experimental strains, the three strains used in Example 6 were used. Samples were prepared from these three strains at time points similar to those in Example 6.

At least three POX gene groups of the present invention (e.g., R2184, R2576, R2693, and the like), a control gene (e.g., conventional POX genes), other marker genes and the like were bound and immobilized onto DNA microarrays. DNAs were immobilized in accordance with a method described in http://cmgm.stanford.edu/pbrown, and the like.

Next, mRNAs were isolated from total RNA prepared above (Qiagen Midi Kit, Chatsworth, Calif.), and transcribed using Superscript II reverse transcriptase (Life Technologies, Grand Island, N.Y.) and oligo(dT) in accordance with a method recommended by the manufacturers. The resultant cDNAs were treated with one unit of RNase H for 30 minutes at 37° C., and purified using a Centricon-30 spin filtration column (Amicon, Beverly, Mass.) to condense to less than 20 μl. One tenth of this amount of the cDNAs were labeled by a random primer polymerization reaction using Cy-3 labeled dUTP or Cy-5 labeled dUTP (each available from Amersham). To perform random primary polymerization, briefly, cDNA was added to 20 μl of the labeled reaction mixture (2 μl of 10×Klenow buffered solution (United States Biochemical)), 0.5 μl of fluorescent dUTP (25 nmol), 3 μl of random primer (Life Technologies), 2 μl of 250 μM dATP, dCTP and dGTP each and 90 μl of dTTP, and one unit of Klenow enzyme (United States Biochemical). After 3-hour incubation at 37° C., two (i.e., Cy-3 and Cy-5) reaction products were combined, and condensed and purified using a Centricon-30 spin filtration column. Thereafter, this sample was lyophilized, and dissolved in 14 μl of hybridization buffered solution (supra), followed by thermal denaturation at 99° C. The resultant sample was applied to DNA microarrays.

The thus-prepared fluorescent hybridization probes were added to DNA microarrays on which the POX gene groups were immobilized. The microarrays were covered with 22×22 mm² Hybrislip (Research Products International). Thereafter, these slides were placed in a waterproof hybridization chamber, followed by hybridization in a water bath at 65° C. for 12 to 16 hours. After the hybridization, the slides were washed in 1×SSC containing 0.03% SDS, and then in 0.2×SSC and 0.05×SSC. The slides were scanned by Scan Array 3000(GSI Lumonics, Oxnard, Calif.). Scanning was also carried out for the slides before the hybridization.

Thereafter, the resultant raw data on the fluorescence was subjected to correction. When there was little change in an expression amount over time, it is not assumed that a total gene expression amount fluctuate. A correction coefficient was calculated from the total or median of fluorescence signals obtained from spots so as to correct fluorescence strength (referred to as global normalization) (Reference 59). Alternatively, in the case where the expression amount of one kind of cell can be considered to be different, such as a comparison of different lineages, a correction coefficient was calculated from a fluorescence signal from a gene, such as a housekeeping gene, whose expression amount is constant. Further, a method using an internal reference at the time of fluorescence labeling may be used.

Based on the thus-obtained corrected data, the relationship between the POX gene groups of the present invention, and the transition of a change in expression of a gene pattern caused by infection with rice blast fungus can be simultaneously analyzed.

(Effect of the Invention)

As described above, all peroxidase (POX) genes belonging to B1 and B2 were constitutively expressed in roots in 5-day-old and 16-day-old seedlings. Except for prxRPA, these genes were constitutively expressed in aerial parts at various levels. According to these results, the present inventors speculate that genes belonging to B1 and B2 are involved in the developmentally regulated basic metabolism and stress-responsive reactions in plants.

R2184 and C52903 belong to B1. These two POXs have putative N-terminal signal peptides and C-terminal extensions. This structure suggests that these POXs are localized in vacuoles. In contrast, R2693, prxRPA and R2576 belonging to B1, and R2329 and S11222 belonging to B2 have only putative N-terminal signal peptides. Therefore, the latter are released outside cells, i.e., they are suggested to be apoplastic peptides. Wound stresses and paraquat treatment induced the expression of both apoplastic (R2329, prx2576 and R2576) and vacuolar (e.g., R2184 and C52903) POXs. Therefore, it is suggested a plurality of POXs having different characteristics, including both vacuolar POXs and apoplastic (i.e., extracellular secretory) POXs, function differently or cooperatively in the same physiological reactions. One of the reasons why a variety of POXs are contained in a single plant is considered to be that plant physiology has to be controlled in such a subtle manner.

### INDUSTRIALLY APPLICABILITY

The disclosure the present invention provides a set of peroxidase (POX) genes useful for evaluation of the characteristics of any plants including plant varieties of the family rice. Further, various POX genes and promoters therefor having a variety of expression specificities are provided. These genes and promoters are useful as materials for modification of plants to confer desired characteristics. In the present invention, the genes of the present invention and promoters thereof can be used to analyze gene expression in plants.

### REFERENCES

(1) Welinder K. G. et al. (1992), Curr. Opin. Struct. Biol., 2:388

(2) Whetten R. et al. (1995), Plant Cell 7, 1001-1013

(3) Espelie K. E. et al. (1986), Plant Physiol. 81, 487-492

(4) Fry S. C. (1986), Ann. Rev. Plant. Physiol. 37, 165-186

(5) Hinman R. L. et al. (1965), Biochemistry 4, 144-158

(6) Chittoor J. M. et al. (1999), Pathogenesis-Related Proteins in Plants (Datta and Muthukrishnan Ed.) 171-193, CRC Press, Boca Raton

(7) Amaya I. et al. (1999), FEBS Lett. 457, 80-84

(8) Abeles F. B. et al. (1988), Plant Physiol. 87. 609-615

(9) Horton R. F. et al. (1993), J. Plant Physiol. 141: 690

(10) Chittoor J. M. et al. (1997), Mol. Plant-Microbe Interact. 7: 861-867

(11) Ito H. et al. (1991), Agric. Biol. Chem. 55: 2445-2454

(12) Ito H. et al. (1994), Plant Cell Rep. 13: 361-366

(13) Langrimini L. M. et al. (1987), Plant Physiol. 84: 438-442

(14) Oohashi Y. (1975), Doctoral dissertation in the Agricultural Chemistry field in the Department of Agriculture, Graduate School of Nogoya University

(15) Yamamoto K. et al. (1997), Plant Mol. Biol. 35: 135-144

(16) Østergaard L. et al. (1998), FEBS Lett. 433: 98-102

(17) Pearson et al. (1988), PNAS 85: 2444-2448

(18) Tijssen (1993), Laboratory Technniques In Biochemistry And Molecular Biology-Hybridization With Nucleic Acid Probes Part I, Second Chapter “Overview of principles of hybridization and the strategy of nucleic acid probe assay”, Elsevier, N.Y.

(19) Nagel et al. (1990), Microbiol. Lett., 67, 325

(20) Gelvin et al. Ed. (1994), Plant Molecular Biology Manual (Kluwer Academic Press Publishers)

(21) Shimamoto et al. (1989), Nature, 338: 274-276

(22) Rhodes et al. (1989), Science, 240: 204-207

(23) Christou et al. (1991), Bio/Technology 9: 957-962

(24) Datta et al. (1990), Bio/Technology 8: 736-740

(25) Moore et al. Current Biology(1995),Vol. 5,737-739

(26) Nagamura et al. (1997), Nogyo-Seibutsu-Sigen-Kenkyujo News [News from National Institute of Agricultural Research], No.57, 1-2

(27) Yoshida K. et al. (1998), Syokubutu-no-Kagaku-Tyosetsu [Chemical Control of Plants], Vol. 33, No. 2, 121-125

(28) Kawaoka A. et al. (1998), Syokubutu-no-Kagaku-Tyosetsu [Chemical Control of Plants], Vol. 33, No. 2, 125-127

(29) Kwak S-S. et al. (1998), Syokubutu-no-Kagaku-Tyosetsu [Chemical Control of Plants], Vol. 33, No. 2, 127-130

(30) Hiraga S. et al. (1998), Syokubutu-no-Kagaku-Tyosetsu [Chemical Control of Plants], Vol. 33, No. 2, 131-136

(31) Kimura et al. (1980), J. Mol. Evol. 16: 111-120

(32) Saitou et al. (1987), Mol. Biol. Evol. 5: 729-731

(33) Studier et al. (1988), Mol. Biol. Evol. 5: 729-731

(34) Welinder K. G. (1979), Eur. J. Biochem. 96: 483-502

(35) Nagy et al. (1988), Plant Molecular Biology Manual (Gelvin et al. Ed.), B4:1-29, Kluwer Academic Publiishers, Dordrecht, Netherlands

(36) Ausubel F. A. et al. Ed. (1988), Current Protocols in Molecular Biology, Wiley, New York, N.Y.

(37) Mohan R. et al. (1990), Plant Physiol. 92, 276-280

(38) Schweizer P. et al. (1998), Plant J. 14, 475-481

(39) Schweizer P. et al. (1997), Plant Physiol. 114: 79-88

(40) Babbs C. F. et al. (1989), Plant Physiol. 90: 1267-1270

(41) Murphy T. M. et al. (1990), Physiol. Plant. 78: 247-253

(42) Asada K.(1992), Physiol. Plant. 85:235-241

(43) Mehlhorn H. et al. (1996), FEBS Lett. 378, 203-206

(44) Kvaratskhelia M. et al. (1997), Plant Physiol. 114, 1237-1245

(45) Fambrough D et al. (1999), Cell 97, 727-741

(46) Iyer V R et al., (1999), Science 283: 83-87

(47) Marton M J, (1999), Nat. Med. 4: 1293-1301

(48) Cargill M et al., (1999), Nat. Genet. 22:231-238

(49) Halushka M K et al., (1999) Nat. Genet. 22:239-246

(50) Marshall A et al., (1998) Nat. Biotechnol. 16: 27-31

(51) Ramsay G et al., (1998) Nat. Biotechnol. 16 40-44

(52) Fodor S P et al., (1991) Science 251: 767-773

(53) Lockart D J et al. (1996) Nat. Biotechnol.: 14:1675-1680

(54) Schena M et al. (1996) Science 270: 467-470

(55) Schena M et al. (1996) Proc. Natl. Acad. Sci. (USA) 93: 10614-10619

(56) DeRisi J et al. (1998) Nat. Genet. 14: 457-460

(57) Ermolaeva O et al. (1998) Nat. Genet. 20:19-23

(58) Schenk P M et al. (2000) Proc. Natl. Acad. Sci. (USA) 97: 11655-11660

(59) Wilson M et al. (1999) Proc. Natl. Acad. Sci. (USA) 96:12833-12838

(60) Sambrook J et al. (1987) Molecular Cloning: A Laboratory Manual, 2nd Ed., Cold Spring Harbor Laboratory Press, Cold Spring Harbor, N.Y.

(61) Ise K. et al. (1998) Ikuzatu [Breeding] 38(Special Issue 2): 404-405

1. A set of peroxidase genes useful for evaluation of a characteristic of plants, comprising:
(A1) a subset of root-expression constitutive genes including at least one type of gene selected from the gene group consisting of:
(1) DNA having a sequence of SEQ ID NO: 1, a homolog thereof, or a fragment thereof;
(2) DNA having a sequence of SEQ ID NO: 3, a homolog thereof, or a fragment thereof;
(3) DNA having a sequence of SEQ ID NO: 5, a homolog thereof, or a fragment thereof;
(4) DNA having a sequence of SEQ ID NO: 7, a homolog thereof, or a fragment thereof;
(5) DNA having a sequence of SEQ ID NO: 9, a homolog thereof, or a fragment thereof;
(6) DNA having a sequence of SEQ ID NO: 11, a homolog thereof, or a fragment thereof;
(7) DNA having a sequence of SEQ ID NO: 13, a homolog thereof, or a fragment thereof;
(8) DNA having a sequence of SEQ ID NO: 15, a homolog thereof, or a fragment thereof;
(9) DNA having a sequence of SEQ ID NO: 17, a homolog thereof, or a fragment thereof;
(10) DNA having a sequence of SEQ ID NO: 19, a homolog thereof, or a fragment thereof; and
(11) DNA having a sequence of SEQ ID NO: 21, a homolog thereof, or a fragment thereof;
(A2) a subset of aerial-expression constitutive genes including at least one type of gene selected from the gene group consisting of:
(12) DNA having a sequence of SEQ ID NO: 23, a homolog thereof, or a fragment thereof; and
(13) DNA having a sequence of SEQ ID NO: 25, a homolog thereof, or a fragment thereof;
(B1) a subset of root-expression stress-inducible genes including at least one type of gene selected from the gene group consisting of:
(14) DNA having a sequence of SEQ ID NO: 27, a homolog thereof, or a fragment thereof;
(15) DNA having a sequence of SEQ ID NO: 29, a homolog thereof, or a fragment thereof;
(16) DNA having a sequence of SEQ ID NO: 31, a homolog thereof, or a fragment thereof;
(17) DNA having a sequence of SEQ ID NO: 33, a homolog thereof, or a fragment thereof; and
(18) DNA having a sequence of SEQ ID NO: 35, a homolog thereof, or a fragment thereof;
(B2) a subset of aerial-expression stress-inducible genes including at least one type of gene selected from the gene group consisting of:
(19) DNA having a sequence of SEQ ID NO: 37, a homolog thereof, or a fragment thereof; and
(20) DNA having a sequence of SEQ ID NO: 39, a homolog thereof, or a fragment thereof;
(C) a gene below:
(21) DNA having a sequence of SEQ ID NO: 41, a homolog thereof, or a fragment thereof.

2. A peroxidase gene, wherein the peroxidase gene is any of:
(a) peroxidase DNA having a sequence of positions 50 to 1021 in SEQ ID NO: 3;
(b) peroxidase DNA having a sequence of positions 108 to 1109 in SEQ ID NO: 5;
(c) peroxidase DNA having a sequence of positions 66 to 1046 in SEQ ID NO: 7;
(d) peroxidase DNA having a sequence of positions 71 to 1078 in SEQ ID NO: 9;
(e) peroxidase DNA having a sequence of positions 134 to 1108 in SEQ ID NO: 11;
(f) peroxidase DNA having a sequence of positions 75 to 1058 in SEQ ID NO: 13;
(g) peroxidase DNA having a sequence of positions 136 to 1147 in SEQ ID NO: 15;
(i) peroxidase DNA having a sequence of positions 29 to 997 in SEQ ID NO: 17;
(j) peroxidase DNA having a sequence of positions 14 to 997 in SEQ ID NO: 19;
(k) peroxidase DNA having a sequence of positions 110 to 1090 in SEQ ID NO: 21;
(l) peroxidase DNA having a sequence of positions 53 to 1033 in SEQ ID NO: 23;
(m) peroxidase DNA having a sequence of positions 20 to 982 in SEQ ID NO: 25;
(n) peroxidase DNA having a sequence of positions 81 to 1025 in SEQ ID NO: 29;
(o) peroxidase DNA having a sequence of positions 44 to 1084 in SEQ ID NO: 31;
(p) peroxidase DNA having a sequence of positions 68 to 1114 in SEQ ID NO: 33;
(q) peroxidase DNA having a sequence of positions 31 to 1101 in SEQ ID NO: 35;
(r) peroxidase DNA having a sequence of positions 34 to 1089 in SEQ ID NO: 37;
(s) peroxidase DNA having a sequence of positions 52 to 1062 in SEQ ID NO: 39; and
(t) a gene having a sequence which hybridizes to any one sequence of (a) to (s) under stringent conditions and encoding a peroxidase having the same expression specificity as that of a peroxidase encoded by said one sequence.

3. A peroxidase gene promoter, wherein the promoter is present on an upstream side of a coding region of a peroxidase gene having a sequence selected from the group consisting of SEQ ID NOs: 1, 3, 5, 7, 9, 11, 13, 15, 17, 19, 21, 23, 25, 27, 29, 31, 33, 35, 37 and 39, or a peroxidase gene which hybridizes to said peroxidase gene under stringent conditions and has the same specific expression activity as that of said peroxidase gene.

4. A peroxidase gene promoter, wherein the promoter is present on an upstream side of a coding region of a peroxidase gene having a sequence selected from the group consisting of SEQ ID NOs: 1, 3, 5, 7, 9, 11, 13, 15, 17, 19, 21, 27, 29, 31, 33 and 35, or a peroxidase gene which hybridizes to said peroxidase gene under stringent conditions and has root-specific expression activity.

5. A peroxidase gene promoter, wherein the promoter is present on an upstream side of a coding region of a peroxidase gene having a sequence selected from the group consisting of SEQ ID NOs: 15, 17, 19 and 21, or a peroxidase gene which hybridizes to said peroxidase gene under stringent conditions and has expression activity in root and aerial parts.

6. A peroxidase gene promoter, wherein the promoter is present on an upstream side of a coding region of a peroxidase gene having a sequence selected from the group consisting of SEQ ID NOs: 23, 25, 37 and 39, or a peroxidase gene which hybridizes to said peroxidase gene under stringent conditions and has aerial-specific expression activity.

7. A peroxidase gene promoter, wherein the promoter is present on an upstream side of a coding region of a peroxidase gene having a sequence selected from the group consisting of SEQ ID NOs: 1, 3, 5, 7, 9, 13, 15, 17, 21, 23 and 25, or a peroxidase gene which hybridizes to said peroxidase gene under stringent conditions and has constitutive expression activity.

8. A peroxidase gene promoter, wherein the promoter is present on an upstream side of a coding region of a peroxidase gene having a sequence selected from the group consisting of SEQ ID NOs: 11 and 19, or a peroxidase gene which hybridizes to said peroxidase gene under stringent conditions and has stress reducible expression activity.

9. A peroxidase gene promoter, wherein the promoter is present on an upstream side of a coding region of a peroxidase gene having a sequence selected from the group consisting of SEQ ID NOs: 27, 29, 31, 33, 35, 37 and 39, or a peroxidase gene which hybridizes to said peroxidase gene under stringent conditions and has stress-inducible expression activity.

10. A method for producing an expression cassette, comprising the steps of:
(1) providing:
(a) a peroxidase gene containing a sequence selected from the group consisting of SEQ ID NO: 1, 3, 5, 7, 9, 11, 13, 15, 17, 19, 21, 23, 25, 27, 29, 31, 33, 35, 37 and 39; and
(b) a peroxidase gene which hybridizes to said peroxidase gene under stringent conditions and has the same specific expression activity as that of said peroxidase gene;
(2) specifying a region having a promoter activity on an upstream side of a coding region of peroxidase gene (a) or (b); and
(3) operatively linking the specified region having the promoter activity to a heterologous gene.

11. A method for analyzing a characteristic of a plant using a set of peroxidase genes according to claim 1, comprising the steps of:
extracting RNA from a sample;
binding the RNA to a membrane;
labeling the set of peroxidase genes according to claim 1;
incubating the membrane along with the set of the labeled peroxidase genes; and
detecting signals derived from the labeled peroxidase genes.

12. A method for analyzing a characteristic of a plant using a peroxidase gene according to claim 2, comprising the steps of:
extracting RNA from a sample;
binding the RNA to a membrane;
labeling the peroxidase gene according to claim 2;
incubating the membrane along with the labeled peroxidase gene; and
detecting signals derived from the labeled peroxidase gene.

13. A method according to claim 12, wherein the characteristic is response to rice blast fungus.

14. A method according to claim 12 or 13, wherein the gene is at least one gene selected from the group consisting of SEQ ID NOs: 29, 31, 33 and 37.

15. A method according to any one of claims 11 to 14, wherein the sample is derived from a plant of the family rice.

16. A method for analyzing a characteristic of a plant using a sequence derived from a promoter according to any one of claim 3 to 9, comprising the steps of:
extracting RNA from a sample;
binding the RNA to a membrane;
labeling an oligonucleotide having the sequence derived from a promoter according to any one of claim 3 to 9;
incubating the membrane along with the labeled oligonucleotide; and
detecting a signal derived from the labeled oligonucleotide.

17. A method for analyzing gene expression using a DNA microarray, comprising the steps of:
(a) immobilizing a set of peroxidase genes according to claim 1 on the DNA microarray;
(b) preparing at least two samples from a plant;
(c) labeling the samples;
(d) mixing and hybridizing the labeled samples to the DNA microarray; and
(e) washing the hybridized DNA microarray and detecting a signal derived from the label.

18. A method for analyzing gene expression using a DNA microarray, comprising the steps of:
(a) immobilizing a peroxidase gene according to claim 2 on the DNA microarray;
(b) preparing at least two samples from a plant;
(c) labeling the samples;
(d) mixing and hybridizing the labeled samples to the DNA microarray; and
(e) washing the hybridized DNA microarray and detecting a signal derived from the label.

19. A method for analyzing gene expression using a DNA microarray, comprising the steps of:
(a) immobilizing an oligonucleotide having the sequence derived from the promoter according to any one of claims 3 to 9 on the DNA microarray;
(b) preparing at least two samples from a plant;
(c) labeling the samples;
(d) mixing and hybridizing the labeled samples to the DNA microarray; and
(e) washing the hybridized DNA microarray and detecting a signal derived from the label.

20. A method according to any one of claims 17 to 19, further comprising the step of:
(f) correcting the detected signal.

21. A method according to any one of claims 17 to 20, further comprising the step of:
(g) analyzing the detected signal or the corrected signal by an analysis software.
